(19) United States
(12) Patent Application Publication
(10) Pub. No.: US 2025/0085228 A1
(43) Pub. Date: Mar. 13, 2025

(54) METHOD FOR PROVIDING INFORMATION RELATED TO DIAGNOSTIC DEVICE AND SYSTEM USING SAME

(71) Applicant: DiaVision, Inc., Seoul (KR)

(72) Inventors: Sukyong LEE, Yongin-si (KR); Seunghyun KIM, Gwacheonsi (KR)

(21) Appl. No.: 18/948,137

(22) Filed: Nov. 14, 2024

Related U.S. Application Data
(63) Continuation of application No. PCT/KR2023/006583, filed on May 16, 2023.

(30) Foreign Application Priority Data
May 18, 2022 (KR) 10-2022-0061096
May 18, 2022 (KR) 10-2022-0061097
May 18, 2022 (KR) 10-2022-0061098
May 18, 2022 (KR) 10-2022-0061099
Aug. 4, 2022 (KR) 10-2022-0097362
Aug. 4, 2022 (KR) 10-2022-0097363
Aug. 4, 2022 (KR) 10-2022-0097364
Aug. 4, 2022 (KR) 20-2022-0001913
Nov. 24, 2022 (KR) 10-2022-0159680

(51) Int. Cl. G01N 21/78

(52) U.S. Cl. CPC G01N 21/78

(57) ABSTRACT

Disclosed is an in vitro diagnostic device including a housing including a first hole and a second hole, wherein the first hole is configured such that a specimen is inserted thereinto, and at least one pad including a first pad, of which at least a partial area is exposed to an outside through the second hole. The first pad exposed to the outside through the second hole is configured to include a first reaction area, a second reaction area, and at least one sign, and at least one of the first reaction area or the second reaction area is configured to express a reaction result based on a reaction with the specimen.

### CROSS-REFERENCE TO RELATED APPLICATIONS

The present application is a continuation of International Patent Application No. PCT/KR2023/006583, filed on May 16, 2023, which is based upon and claims the benefit of priority to Korean Patent Application Nos. 10-2022-0061096, 10-2022-0061097, 10-2022-0061098, and 10-2022-0061099 filed on May 18, 2022. The disclosures of the above-listed applications are hereby incorporated by reference herein in their entirety.

### BACKGROUND

Embodiments of the present disclosure described herein relate to a method of providing information related to a diagnostic device, and more particularly, relate to a method of analyzing data that may be acquired through a diagnostic device to provide related information to users and manufacturers.

A method of measuring a measurement value of biometric information through a sample (or a specimen) that is collected from a body continues to be developed. Urine or blood is generally used as a sample, and as a measurement method develops, a method of measuring a measurement value of biometric information, such as diabetes, by using sweat or tears as a sample is being developed. Furthermore, a method of measuring a measurement value of biometric information by collecting a sample from saliva and exhaled breathes is also being developed.

As a general method of measuring measurement values of biometric information by using samples, it is possible to simply measure the measurement values of the biometric information through a qualitative analysis for determining whether or not the urine response result to the reagent is positive or negative by visually identifying the urine diagnosis strip.

In measuring measurement values of biometric information, many reagent reactions to various diseases are required, and a quantitative determination is required to determine a degree of condition through measurement values as well as a qualitative analysis of positive/negative reactions.

Various information, such as a blood glucose level, an acidity (pH), and a protein level could be acquired through reaction inspections using blood as well as urine, but the measurement equipment has to be separately provided depending on the type of information that is to be measured. Furthermore, the measurement equipment is medium and large equipment, and the price of the equipment is relatively high, and specialized knowledge is required for the measurement method, and thus, only some experts, such as doctors and clinical pathologists, may access it limitedly.

To solve this problem, the development of home-testing and portable measuring equipment that may be easily used by the general public has become active, and recently, a method of measuring biometric information by inputting sample information into a mobile terminal, such as a smartphone, has been studied.

Recently, a technology for analyzing a strip, in which the sample is collected, through an optical image sensor, such as a camera included in the terminal, has been developed with much interest, and to accurately analyze a reaction between a sample pad and a sample attached to the strip, a method of analyzing and correcting a degree of discoloration of the sample pad to acquire an accurate degree of discoloration is being actively studied.

When a diagnostic device is used to measure a measurement value of biometric information, various information related to the diagnostic device exists. In addition, there are various information that may be derived from the result data acquired through the method. Accordingly, there is a need for a system that may integrally manage or provide the information.

### SUMMARY

An aspect of the present disclosure relates to a method capable of providing information related to a diagnostic device to a user terminal and a manufacturer server.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1A is a view illustrating an example of an in vitro diagnostic system according to various embodiments.

FIG. 1B is a view illustrating another example of an in vitro diagnostic system according to various embodiments.

FIG. 2A is a view illustrating an example of an in vitro diagnostic device according to various embodiments.

FIG. 2B is a view illustrating various examples of a structure and/or configuration that constitutes an exterior of an in vitro diagnostic device according to various embodiments.

FIG. 2C is a view illustrating examples of various types of visual markers that are formed in a housing of an in vitro diagnostic device according to various embodiments.

FIG. 3 is an example of an exploded perspective view of an in vitro diagnostic device according to various embodiments.

FIG. 4A is a cutting view for observing a side surface of an in vitro diagnostic device with respect to X1-X1′ according to various embodiments.

FIG. 4B is a view illustrating another example of a position of at least one sign of an in vitro diagnostic device according to various embodiments.

FIG. 5 is a view illustrating an example of an in vitro diagnostic device that is configured to include a plurality of pads according to various embodiments.

FIG. 6 is a view illustrating an example of use of an in vitro diagnostic system according to various embodiments.

FIG. 7A is a view illustrating an example of a holder according to various embodiments.

FIG. 7B is a view illustrating examples of a support part according to various embodiments.

FIG. 7C is a view illustrating an example of a state, in which an electronic device is held on a holder according to various embodiments.

FIG. 8 is a view illustrating an example of a holder, in which an anti-reflection area is formed, according to various embodiments.

FIG. 9 is a view illustrating an example of a holder including a member for guiding disposition of an in vitro diagnostic device according to various embodiments.

FIG. 10A is a view illustrating an example of a holder, in which an upper housing and a lower housing are configured to be at least partially connected to each other, according to various embodiments.

FIG. 10B is a view illustrating an example of being mounted on an electronic device by an upper housing according to various embodiments.

FIG. 11 is a view illustrating an example of a holder according to various embodiments.

FIG. 12A is a view illustrating examples of observing a holder for an expert from various directions according to various embodiments.

FIG. 12B is a view illustrating an example of configurations of a holder for an expert according to various embodiments.

FIG. 13 is a view illustrating an example of an electronic device that is mounted on a holder for an expert according to various embodiments.

FIG. 14 is a view illustrating an example of configurations of an electronic device according to various embodiments.

FIG. 15 is a flowchart illustrating operations based on various functions that are provided in a process of providing a diagnosis result by using an image of an in vitro diagnostic device by an electronic device, according to various embodiments.

FIG. 16 is a view illustrating various functions that are provided by an electronic device, according to various embodiments.

FIG. 17 is a flowchart illustrating a method of providing a timer function by an electronic device based on product information, according to various embodiments.

FIG. 18 is a view illustrating a method of providing a timer function by an electronic device based on product information, according to various embodiments.

FIG. 19 is a flowchart illustrating a method of providing a plurality of timer functions by an electronic device based on product information, according to various embodiments.

FIGS. 20A, 20B, and 20C illustrate screens for providing a plurality of timer functions based on product information by an electronic device, according to various embodiments.

FIG. 21 is a flowchart illustrating a method, in which an electronic device provides a continuous photographing function based on a timer, according to various embodiments.

FIG. 22 illustrates screens, in which an electronic device provides a continuous photographing function based on a timer, according to various embodiments.

FIG. 23 is a flowchart illustrating a method of guiding a photographing position and/or a photographing angle of an in vitro diagnostic device by an electronic device, according to various embodiments.

FIG. 24 is a flowchart illustrating an example of a method of guiding a photographing position and/or a photographing angle of an in vitro diagnostic device by an electronic device, according to various embodiments.

FIG. 25 illustrates screens, in which an electronic device guides a photographing position and/or a photographing angle of an in vitro diagnostic device, according to various embodiments.

FIG. 26 illustrates various implementation examples of a second guide object according to various embodiments.

FIG. 27 is a flowchart illustrating an example of a method of guiding a photographing position and/or a photographing angle of an in vitro diagnostic device by an electronic device, according to various embodiments.

FIG. 28 illustrates screens, in which an electronic device guides a photographing position and/or a photographing angle of an in vitro diagnostic device, according to various embodiments.

FIG. 29 is a flowchart illustrating a method of guiding a disposition direction of an in vitro diagnostic device by an electronic device, according to various embodiments.

FIG. 30A is a view illustrating a method of guiding a disposition direction of an in vitro diagnostic device by an electronic device, according to various embodiments.

FIG. 30B illustrates a screen, in which an electronic device guides a disposition direction of an in vitro diagnostic device according to various embodiments.

FIG. 31 illustrates examples of at least one object that is displayed based on a disposition direction of an electronic device, according to various embodiments.

FIG. 32 is a flowchart illustrating a method of providing result information based on at least one inspection line by an electronic device, according to various embodiments.

FIGS. 33A and 33B are views illustrating a method of acquiring result information based on at least one inspection line by an electronic device, according to various embodiments.

FIG. 33C illustrates screens, in which an electronic device provides result information based on at least one inspection line, according to various embodiments.

FIG. 33D illustrates another example of a graphic object that emphasizes at least one inspection line, according to various embodiments.

FIG. 34 is a flowchart illustrating an example of an operation of an electronic device according to various embodiments.

FIG. 35 is a view illustrating an example of an absolute angle and a relative angle according to various embodiments.

FIG. 36 is a flowchart illustrating an example of an operation of an electronic device according to various embodiments.

FIG. 37 is a flowchart illustrating an example of an operation of an electronic device according to various embodiments.

FIG. 38 is a flowchart illustrating an example of an operation of an electronic device according to various embodiments.

FIG. 39 is a view illustrating an example of an operation of performing photographing while an area, in which, a focus of an electronic device is set, is moved according to various embodiments.

FIG. 40 is a flowchart illustrating an example of an operation of an electronic device according to various embodiments.

FIG. 41 is a flowchart illustrating an operation of an electronic device according to various embodiments.

FIG. 42 is a view illustrating an example of an operation of photographing a plurality of pads of an electronic device according to various embodiments and an operation of providing information on an in vitro diagnostic result based on information that is analyzed for each of the plurality of pads.

FIG. 43A is a view illustrating an example of a guide UI based on a portrait mode that is provided by an electronic device according to various embodiments.

FIG. 43B is a view illustrating an example of a guide UI based on a landscape mode that is provided by an electronic device according to various embodiments.

FIG. 44 is a flowchart illustrating an operation of an electronic device according to various embodiments.

FIG. 45 is a flowchart illustrating an operation of an electronic device according to various embodiments.

FIG. 46 is a view illustrating an example of an operation of performing a function based on different modes depending on whether an electronic device is gripped (or held) according to various embodiments.

FIG. 47A is a view illustrating an example of a holder holding mode of an electronic device according to various embodiments.

FIG. 47B is a view illustrating an example of a user grip mode of an electronic device according to various embodiments.

FIG. 48 is a flowchart illustrating an operation of an electronic device according to various embodiments.

FIG. 49 is an environmental diagram of an information providing system related to a diagnostic device according to an embodiment.

FIG. 50 is a block diagram of a system for providing information related to a diagnostic device according to an embodiment.

FIG. 51 is a flowchart of a method of providing information related to a diagnostic device according to an embodiment.

FIG. 52 is a flowchart of a method for generating healthcare solution data according to an embodiment.

FIG. 53 is a flowchart of a method for generating usage prediction data according to an embodiment.

### DETAILED DESCRIPTION

A method of providing information related to a diagnostic device performed by at least one processor according to an embodiment may include an operation of acquiring user information and specification information of the diagnostic device, an operation of transmitting use manual data of the diagnostic device to a user terminal based on the specification information of the diagnostic device above, an operation of acquire result data of the diagnostic device from the user terminal above, an operation of generating healthcare solution data based on the user information and the result data of the diagnostic device and transmitting them to the user terminal, and an operation of generating usage prediction data of the diagnostic device based on the user information and the healthcare solution data and transmitting them to a manufacturer server.

Here, the user information may include at least one of the gender, the age, and the area of the user, information on diseases, information on a medication, interested diagnosis items, attending hospital information, and health diagnosis results, and the specification information of the diagnostic device may include at least one of the name, the manufacturer, and possible diagnosis items of the diagnostic device.

Here, the result data of the diagnostic device may be an image that is obtained by photographing the diagnostic device, into which a specific is inserted.

Here, the use manual data of the diagnostic device may include timer information corresponding to the specification information of the diagnostic device, and the image may be an image that is taken after a time corresponding to the timer information exceeds from a time point, at which a first input corresponding to a start of the timer is acquired.

Here, the operation of transmitting the use manual data of the diagnostic device may include an operation of providing a notification to the user through a user terminal when a time corresponding to the timer information exceeds from the time point, at which the first input was acquired.

Here, the operation of generating the healthcare solution data may include an operation of extracting reaction area from the result data of the diagnostic device result data and generating reaction result data from the reaction area, and an operation of inputting the user information and the reaction result data into an artificial intelligence model to generate healthcare solution data including a self-diagnosis cycle using an exercise therapy, a diet therapy, and a diagnostic device.

Here, the reaction result data may include at least one of negative/positive results and values of the diagnosis item through an analysis of colors, through extraction of an inspection line from the reaction area and an analysis of pixel values.

Here, the operation of generating the usage prediction data of the diagnostic device may include an operation of calculating a usage of the diagnostic device for a specific period for all of a plurality of users based on the self-diagnosis cycles of the plurality of users.

Here, the operation of generating the usage prediction data of the diagnostic device may include an operation of setting a delivery schedule for the diagnostic device based on a delivery period depending on residential area information of the user included in the user information and a self-diagnosis date that is calculated depending on the self-diagnosis cycle.

Here, the method may further include an operation of identifying whether the diagnostic device is defective from the result data of the diagnostic device and analyzing a cause of the defect to generate defect-related data, and an operation of transmitting the defect-related data to the manufacturer server.

Here, the operation of acquiring the result data of the diagnostic device may include an operation of acquiring result data of a first diagnostic device at a first time point, result data of a second diagnostic device at a second time point, and result data of a third diagnostic device at a third time point, from the user terminal, and the operation of generating the healthcare solution data may include an operation of determining a user health pattern based on the result data of the first diagnostic device, the result data of the second diagnostic device, and the result data of the third diagnostic device, and an operation of generating healthcare solution data depending on the user health pattern based on an analysis result of result data of a fourth diagnostic device that are acquired at the fourth time point from the user terminal.

Here, a computer program that is recorded on a computer-readable recording medium may be provided to execute the method of providing information related to the diagnostic device.

The information providing system related to the diagnostic device according to an embodiment may include a communication part that communicates with the user terminal to acquire user information and specification information of the diagnostic device, transmits use manual data of the diagnostic device based on the specification information of the diagnostic device, and acquires result data of the diagnostic device, a result analyzing part that generates healthcare solution data based on the user information and the result data of the diagnostic device and transmits the healthcare solution data to the communication part to transmit them to the user terminal above, and a usage analyzing part that generates usage prediction data of the diagnostic device based on the user information and the healthcare solution data and transmits the usage prediction data of the diagnostic device to the communication part to transmit them to the user terminal.

The embodiments described in the present specification are intended to clearly describe the spirit of the present disclosure to those skilled in the art, to which the present disclosure pertains, and the present disclosure is not limited to the embodiments described in the specification, and the scope of the present disclosure should be construed as including corrections or modifications that do not depart from the spirit of the present disclosure.

The terms used in the specification are selected as general terms that are currently widely used as possible in consideration of the functions of the present disclosure, but may vary depending on the intention of those skilled in the art, to which the present disclosure pertains, precedents, or the emergence of new technologies. However, in contrast, when a specific term is defined and used in an arbitrary meaning, the meaning of the term will be described separately. Accordingly, the terms used in the specification should be interpreted based on not just the name of the term but the practical meaning of the term and the overall contents of the specification.

The drawings attached to the specification are for easily describing the present disclosure, and the shape illustrated in the drawings may be exaggerated and displayed as necessary to help understanding of the present disclosure, and thus the present disclosure is not limited by the drawings.

In the specification, when it is determined that a detailed description of a known configuration or function related to the present disclosure may obscure the gist of the present disclosure, a detailed description thereof will be omitted as necessary. The electronic device according to various embodiments disclosed in the present disclosure may be various types of devices. The electronic device may include, for example, a portable communication device (e.g., a smartphone), a computer device, a portable multimedia device, a portable medical device, a camera, a wearable device, or a home appliance. The electronic device according to the embodiment of the present disclosure is not limited to the above-described devices.

Various embodiments of this document and the terms used herein are not intended to limit the technical features described herein to specific embodiments and should be understood as including various modifications, equivalents, or substitutes of the corresponding embodiment. In relation to the description of the drawings, similar reference numerals may be used for similar or related components. The singular form of a noun corresponding to an item may include one or a plurality of items unless the context, in which it is related, clearly indicates otherwise. In the present disclosure, phrases, such as “at least one of A or B,” “at least one of A or B,” “A, B or C,” “at least one of A, B or C,” and “at least one of A, B, or C”, may each include any one of the items listed together in the corresponding phrase, among the phrases, or all possible combinations thereof. Terms, such as “first” and” second” may simply be used to distinguish a corresponding component from another corresponding component and do not limit the corresponding component in other aspects (e.g., importance or order). when a certain (e.g., first) component is referred to as “coupled” or “connected” together with or without the term “functional” or “communicatively” in another (e.g., second) component, it means that any one of the components may be connected directly (e.g., by wire), wirelessly, or through a third component.

The term “module” used in various embodiments of the present disclosure may include a unit that is configured in hardware, software, or firmware, and may be used interchangeably with terms, such as logic, logical blocks, components, or circuits, for example. The module may be an integrated component or a minimum unit or a part of the component that performs one or more functions. For example, according to an embodiment, the module may be configured in the form of an application-specific integrated circuit (ASIC).

Various embodiments of the present disclosure may be configured as software (e.g., a program) including one or more instructions that are stored in a storage medium (e.g., an internal memory or an external memory) that may be readable by a device (e.g., an electronic device). For example, a processor of a device (e.g., the electronic device) may call and execute at least one instruction, among one or more instructions that are stored from the storage medium. This makes it possible for the device to be operated to perform at least one function according to the at least one instruction called. The one or more instructions may include codes that are generated by a compiler or codes that are executed by an interpreter. The storage medium that is readable by the device may be provided in the form of a non-transitory storage medium. Here, ‘non-transitory’ means that the storage medium is a tangible device and does not contain a signal (e.g., electromagnetic waves), and this term does not distinguish between cases, in which data is stored semi-permanently in a storage medium, and cases, in which data is stored temporarily.

According to an embodiment, a method according to various embodiments disclosed in the present disclosure may be provided to be included in a computer program product. The computer program product may be traded between a seller and a buyer as a product. The computer program product may be distributed in the form of a device-readable storage medium (e.g., a compact disk read only memory (CD-ROM)) or may be distributed online (e.g., downloaded or uploaded) directly through an application store (e.g., smartphones). In the case of online distribution, at least a portion of the computer program product may be at least temporarily stored in a device-readable storage medium, such as a server of a manufacturer, a server of an application store, or a memory of a relay server.

According to various embodiments, the components (e.g., a module or a program) of the above-described components may include a single or a plurality of entities, and some of the plurality of entities may be separately disposed in other components. According to various embodiments, one or more of the above-described corresponding components or operations may be omitted, or one or more other components or operations may be added. Alternatively or additionally, a plurality of components (e.g., modules or programs) may be integrated into a single component. In this case, the integrated component may perform one or more functions of the plurality of components in the same or similar manner as those performed by the corresponding component, among the plurality of components, prior to the integration. According to various embodiments, operations performed by a module, program, or other component may be sequentially, in parallel, repeatedly, or heuristically executed, or one or more of the operations may be executed in a different order, omitted, or one or more other operations may be added.

According to various embodiments, there may be provided an in vitro diagnostic device including a housing, an introduction member formed in the housing, and into which a sample is introduced, a membrane including a control line and at least one test line, at least portion of which is exposed to an outside through a hole formed in a specific area of the housing, and a color sign configured in at least a portion of the membrane exposed to the outside through the hole, wherein the at least one test line is set to be displayed in a color corresponding to at least one color included in the color sign depending on a reaction based on the introduced sample.

According to various embodiments, there may be provided a box manufactured to accommodate an in vitro diagnostic device, including a body defining an interior space, and including an upper surface, a lower surface, and a plurality of side surfaces connecting the upper surface and the lower surface, wherein, among the plurality of side surfaces, a first side surface and a second side surface facing the first side surface have a structure that may be cut at an angle in a first range from a bottom surface of the body, the angle in the first range is greater than or equal to 10 degrees and less than 20 degrees, a portion, at which the electronic device of the user may be held at the angle in the first range is formed on the remaining portions of the body when a portion of the body is cut by the structure, and a support part that may support the electronic device is formed at at least a portion of the plurality of side surfaces.

According to various embodiments, there may be provided an electronic device including a camera, a display, a memory, and at least one processor, wherein, when being executed, a program stored in the memory causes the at least one processor to acquire identification information for an in vitro diagnostic device by using the camera, display an execution screen including information on a remaining time until a specific time, at which photographing of the in vitro diagnostic device is allowed, and a graphic object that is configured to provide a photographing function based on driving of the timer, wherein the specific time is identified based on the acquired identification information, display a first object that is set to guide a photographing position of a subject together with the at least one first preview image when the graphic object is selected after the specific time has elapsed, acquire a first image of the in vitro diagnostic device by using the camera based on the graphic object corresponding to the in vitro diagnostic device, acquire a second image of the at least one membrane of the in vitro diagnostic device based on the first image, and acquire result information based on at least one of a test line or a control line that is identified from the second image, and display the first image, the result information, and at least one graphic object associated with the result information for emphasizing at least one of the test line and the control line.

According to various embodiments, there may be provided an electronic device including a camera, a display, a memory, and at least one processor, wherein, when being executed, a program stored in the memory causes the at least one processor to acquire product information of an in vitro diagnostic device by using the camera, identify a type of the in vitro diagnostic device based on the product information, display an execution screen including information on a remaining time until a first specific time, at which photographing of a first type of in vitro diagnostic device is allowed, and a first graphic object that is configured to provide a photographing function, based on driving of the timer, when the in vitro diagnostic device is of the first type, and display an execution screen including information on a remaining time until a second specific time, at which photographing of a second type of in vitro diagnostic device is allowed, and a second graphic object that is configured to provide a photographing function, based on the driving of the timer, when the in vitro diagnostic device is of the second type.

According to various embodiments, there may be provided an electronic device including a camera, a display, a memory, and at least one processor, wherein, when being executed, a program stored in the memory causes the at least one processor to identify a remaining time until a specific first time, at which photographing of the in vitro diagnostic device is allowed, and a remaining time until a specific second time, at which photographing of the in vitro diagnostic device is not allowed, based on driving of the timer, determine whether the specific second time has elapsed after the specific first time elapsed, control a state of at least one graphic object for photographing included in the execution screen to an inactive state when the type of execution screen being displayed on the display is of the first type when the specific second time has elapsed, and maintain the display of the execution screen when the type of the execution screen being displayed on the display is of the second type.

According to various embodiments, there may be provided an electronic device including a camera, a display, a memory, and at least one processor, wherein, when being executed, a program stored in the memory causes the at least one processor to identify a remaining time until a specific first time, at which photographing of the in vitro diagnostic device is allowed, and a remaining time until a specific second time, at which photographing of the in vitro diagnostic device is not allowed, based on driving of the timer, determine whether the specific second time has elapsed after the specific first time elapsed, display a graphic object related to the second time on the execution screen being displayed on the display when the specific second time has elapsed.

According to various embodiments, there may be provided an electronic device including a camera, an inertia sensor, a display, and a processor, wherein the processor is configured to acquire at least one preview image by using the camera, display a first object for guiding a photographing position of at least a portion of the in vitro diagnostic device on the display, wherein the first object is displayed such that at least a portion of the at least one first preview image overlaps, acquire a second preview image including at least a portion of the in vitro diagnostic device by using the camera, perform a first function based on that a degree of similarity of at least a portion of the in vitro diagnostic device included in the second preview image to the first object is included within a specific range while an inclination of the electronic device identified by using the inertia sensor satisfies a specific inclination range, and perform a second function being different from the first function based on that the degree of similarity of the at least a portion of the in vitro diagnostic device included in the second preview image to the first object is less than the specific range, and wherein an upper limit value of the specific range is set in association with the specific inclination range.

According to various embodiments, there may be provided an electronic device including a camera, an inertia sensor, a display, and a processor, wherein the processor is configured to acquire at least one preview image by using the camera, display a first object for guiding a photographing position of at least a portion of the in vitro diagnostic device and a second object corresponding to an inclination of the electronic device identified by using the inertia sensor on the display, wherein at least one of the first object or the second object is displayed such that at least a portion of the at least one first preview image overlaps, acquire a second preview image including at least a portion of the in vitro diagnostic device by using the camera, perform a first function based on that a degree of similarity of at least a portion of the in vitro diagnostic device included in the second preview image to the first object is included within a specific range while the second object overlaps at least a partial area of the first object according to movement of the electronic device.

According to various embodiments, there may be provided an electronic device including a camera, a display, and a processor, wherein the processor is configured to acquire at least one preview image by using the camera, display a first object for guiding a photographing position of at least a portion of the in vitro diagnostic device on the display, display the first object such that at least a portion of the at least one first preview image overlaps, acquire the second preview image by using the camera, wherein the second preview image includes at least a portion of the in vitro diagnostic device and at least one object located in the housing of the in vitro diagnostic device within a specified distance from the at least a portion of the in vitro diagnostic device, acquire position information of the at least one object of the in vitro diagnostic device, and display at least one third object corresponding to the at least one object based on the acquired position information, and perform a first function based on that a degree of similarity of at least a portion of the in vitro diagnostic device to the first object is included within a specific range, and the at least one third object overlaps the at least one object, wherein the at least one third object is displayed at a first position with respect to the first object when the in vitro diagnostic device is of a first type, and is displayed at a second position being different from the first position with respect to the first object when the in vitro diagnostic device is of a second type being different from the first type.

According to various embodiments, there may be provided an electronic device including a camera, a display, a memory, and at least one processor, wherein, when being executed, a program stored in the memory causes the at least one processor to acquire a first image by using the camera, wherein the first image includes an in vitro diagnostic device including at least one membrane, acquire a second image of the at least one membrane of the in vitro diagnostic device based on the first image, acquire result information based on at least one of a test line or a control line identified from the second image, and display the first image, the result information, and at least one graphic object associated with the result information for emphasizing at least one of the test line and the control line.

According to various embodiments, there may be provided an electronic device including a camera, a display, and a processor, wherein the processor is configured to acquire at least one first image through the camera in a first state, in which at least a portion of the housing of the in vitro diagnostic device is focused, identify a position of the membrane of the in vitro diagnostic device located on a plane being different from that of the housing based on the at least one first image acquired, acquire a second image including the membrane in a second state, in which at least a portion of the membrane is focused based on that the position of the membrane is identified, and output a biometric information measurement result through the in vitro diagnostic device based on the acquired second image.

According to various embodiments, there may be provided an electronic device including a camera, a display, and a processor, wherein the processor is configured to acquire at least one preview image by using the camera, display a first object for guiding a first photographing position of the first membrane, a second object for guiding a second photographing position of the second membrane, and a movement guide, wherein the first object and the second object are displayed to overlap at least a portion of the at least one preview image, the first object is displayed in a first position, the second object is displayed in a second position corresponding to a direction indicated by the movement guide with respect to the first object, acquire a first image including at least a portion of the first membrane and the second membrane when the first membrane is aligned with the first object displayed in the first position according to the movement of the electronic device, display the second object displayed at the second position based on acquisition of the first image, acquire a second image including at least a portion of the first membrane and the second membrane when the second membrane is aligned with the second object in the first position according to the movement of the electronic device, and identify biometric information corresponding to the first membrane and the second membrane based on the first image and the second image.

According to various embodiments, there may be provided an electronic device including a camera, a display, and a processor, wherein the processor is configured to acquire a first image for the first membrane, and acquire a second image for the second membrane, identify first biometric information corresponding to the first membrane, and identify second biometric information corresponding to the second membrane, and acquire result information based on a comparison of the first biometric information and the second biometric information.

According to various embodiments, there may be provided an electronic device including a flash, a camera, an inertia sensor, a display, and a processor, wherein the processor is configured to acquire at least one preview image by using the camera, display an execution screen including a first object for guiding a photographing position of at least a portion of the in vitro diagnostic device on the display, and acquire at least one image of the in vitro diagnostic device by using the camera while driving the flash based on a condition associated with an inclination associated with at least one of the electronic device or the in vitro diagnostic device is satisfied while the execution screen is displayed.

According to various embodiments, there may be provided an electronic device including a camera, a memory, and at least one processor, wherein, when being executed, a program stored in the memory causes the at least one processor to identify a specific photographing mode, among a plurality of photographing modes for photographing an in vitro diagnostic device of the electronic device, wherein the plurality of photographing modes include a first photographing mode, in which photographing is performed while the electronic device is gripped by a user, and a second photographing mode in which photographing is performed while the electronic device is gripped by a holder, provide a first screen for guiding photographing of the in vitro diagnostic device when the photographing mode of the electronic device is the first photographing mode, provide a first function for photographing the in vitro diagnostic device, provide a second screen for guiding photographing of the in vitro diagnostic device when the photographing mode of the electronic device is the second photographing mode, and provide a second function for photographing the in vitro diagnostic device.

According to various embodiments, there may be provided an electronic device including a camera, a display, and a processor, wherein the processor is configured to acquire at least one preview image by using the camera, display a first object for guiding a first photographing position of the first membrane, a second object for guiding a second photographing position of the second membrane, and display a movement guide, wherein the first object and the second object are displayed to overlap at least a portion of the at least one preview image, the first object is displayed in a first position, the second object is displayed in a second position corresponding to a direction indicated by the movement guide with respect to the first object, acquire a first image including at least a portion of the first membrane and the second membrane when the first membrane is aligned with the first object displayed in the first position according to the movement of the electronic device, display the second object displayed at the second position at the first position based on acquisition of the first image, acquire a second image including at least a portion of the first membrane and the second membrane when the second membrane is aligned with the second object in the first position according to the movement of the electronic device, and identify biometric information corresponding to the first membrane and the second membrane based on the first image and the second image.

Hereinafter, an example of an in vitro diagnostic system 1 according to various embodiments will be described.

The in vitro diagnostic system 1 according to various embodiments may be a system that is configured to provide information on an in vitro diagnosis result based on an electronic analysis of an in vitro diagnostic device (an in vitro diagnostic device (IVD) (or a bio sensor) 110). For example, the in vitro diagnostic system 1 electronically analyzes information on physiological or pathological conditions, diseases, treatment responses, and/or treatment results (hereinafter referred to as in vitro diagnostic results) that are to be diagnosed (or observed, or informed) by using the in vitro diagnostic device 110 based on an image of the in vitro diagnostic device 110, which is acquired based on photographing of the in vitro diagnostic device 110 by using an electronic device 130 of the user, and may provide it to the user of the in vitro diagnostic system 1. When the user visually identifies the in vitro diagnosis result that is expressed (e.g., a test line on a pad (or a membrane)) by the in vitro diagnostic device 110, there is a high risk of misjudgment, and the costs may be high and a convenience of use may be low to analyze the in vitro diagnostic device 110 by using specialized inspection equipment. In contrast, the in vitro diagnostic system 1 according to various embodiments may improve an accuracy of result information based on an electronic analysis method, and may improve the convenience of use and reduce the costs by allowing the user to conveniently use the in vitro diagnostic system 1 by using the electronic device 130 used by the user. Meanwhile, the in vitro diagnostic system 1 according to various embodiments may be configured to provide a function for analyzing the in vitro diagnostic device 110 more conveniently and accurately by using the electronic device 130, and this will be described with reference to various embodiments described below.

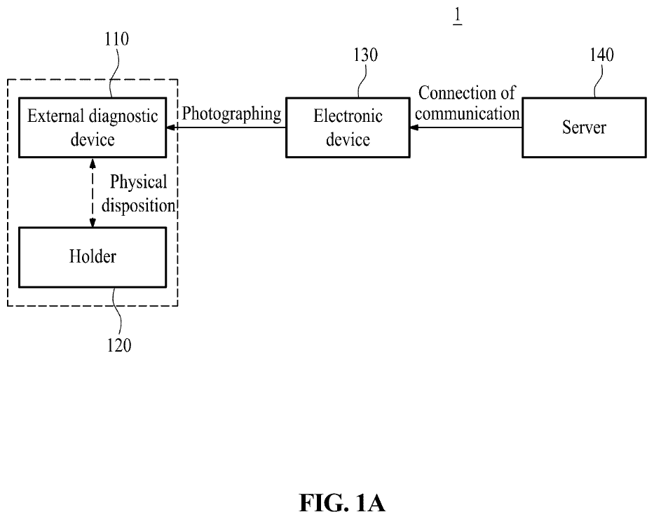

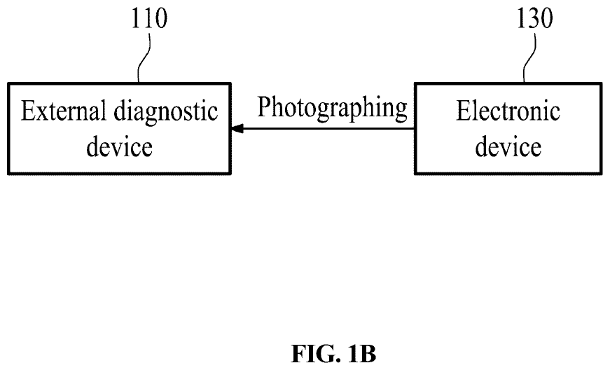

FIG. 1A is a view illustrating an example of the in vitro diagnostic system 1 according to various embodiments. FIG. 1B is a view illustrating another example of the in vitro diagnostic system 1 according to various embodiments. Hereinafter, an example of the in vitro diagnostic system 1 will be described with reference to FIGS. 1A and 1B.

Referring to FIG. 1A, according to various embodiments, the in vitro diagnostic system 1 may include an in vitro diagnostic device 110, a holder 120, an electronic device 130, and a server 140.

According to various embodiments, the in vitro diagnostic device 110 may include inspection materials (e.g., reagents, controls/correction substances) for ex vivo inspection of specimens (or samples) (e.g., saliva, urine, feces, blood, and tissue cells) derived from humans or animals, and may be a mechanism (or a machine) that is configured to express (or provide) the in vitro diagnosis result in a form (visually) that may be conveniently visually recognized by the user of the in vitro diagnostic device 110 according to the inspection. For example, the in vitro diagnosis result may include results that are related to diagnosis and prognosis of diseases, determination of health conditions, determination of disease treatment effectiveness, and/or prevention, but is not limited thereto and may include recommended information for health promotion (wellness recommendation information) that is determined based on information (e.g., the time-specific in vitro diagnosis result (history)) and in vitro diagnosis results in the form of processing and/or analyzing the described examples. The in vitro diagnostic device 110 may be configured to perform an inspection based on an immunochemical diagnosis method for diagnosing and tracking a disease by using an antigen/antibody reaction, a molecular diagnosis method for testing nucleic acids (DNA and RNA) containing genetic information, a plasma diagnosis method, a blood diagnosis method, and/or a clinical microbiological diagnosis method, and a well-known diagnosis method that is not limited to the examples described (e.g., configured to include (or receive) inspection materials of the corresponding diagnosis method). Hereinafter, for convenience of description, an in vitro diagnostic device 110 (an in vitro diagnostic device for diagnosing Corona virus (COVID-19)) for an inspection based on an immunochemical diagnosis method will be described as an example, but it is obvious to those skilled in the art that the various embodiments described below are not limited to the examples described and may be applied to the in vitro diagnostic device 110 that is configured for various types of diagnostic methods.

According to various embodiments, the holder 120 may be a mechanism (or structure) for holding the electronic device 130 of the user. A user using the in vitro diagnostic system 1 may photograph the in vitro diagnostic device 110 by using the electronic device 130 while gripping the electronic device 130 directly, but may photograph the in vitro diagnostic device 110 while the electronic device 130 is held in the holder 120. The holder 120 may include a holder for a general user that is configured to be conveniently used by a user (or a buyer) of the in vitro diagnostic device 110, and a holder for an expert that is configured to be used in a specialized institution, such as a medical institution, and this will be described later with reference to the drawings.

According to various embodiments, the electronic device 130 may be an electronic device that is usable by a user and is configured to include an electronic component (or device) (e.g., a camera, and/or an image sensor) for photographing. For example, the electronic device 130 may include a smartphone, a wearable device, a head mounted display (HMD) device, and the like, but is not limited to the examples described and may include various types of electronic devices that may be used by the user. As will be described later, the electronic device 130 may store an application that is configured to provide a function of analyzing the in vitro diagnostic device 110 (e.g., download and store an application from an application distribution server (not illustrated), may provide an execution screen (or a user interface (UI)) for photographing the in vitro diagnostic device 110 based on the execution of the application, and may perform a function of providing information on the in vitro diagnosis result based on the captured image of the in vitro diagnostic device 110. As described above, the in vitro diagnosis result provided based on an application may include results that are related to diagnosis and prognosis of diseases, determination of health conditions, determination of disease treatment effectiveness, and/or prevention, but is not limited thereto and may include recommended information for health promotion (wellness recommendation information, digital vaccines, and digital medicines) that is determined based on information (e.g., the time-specific in vitro diagnosis result (history)) and in vitro diagnosis results in the form of processing and/or analyzing the described examples. Furthermore, the application may be configured to store history information on the in vitro diagnosis results, and may perform a function of providing at least one piece of information for health promotion based on the stored history information. In the specification, a biometric information measurement value that is analyzed by the electronic device 130 means a concentration and/or amount of the object (e.g., an antigen, an antibody) to be inspected by the in vitro diagnostic device 110, and this may be used to determine the above-described in vitro diagnosis result.

According to various embodiments, the server 140 may be various types of external electronic devices that are configured outside the electronic device 130. The server 140 may include at least one of a distribution server for providing an application (or a program) that is configured to provide an analysis service for the in vitro diagnostic device 110 to the electronic device 130, an analysis server for providing the in vitro diagnostic device 110 based on analysis of an image for the in vitro diagnostic device 110 that is received from the electronic device 130, a storage server that is configured to store various type of information (e.g., user account information, or information on in vitro diagnosis results for user account information), or a learning server for performing learning based on the stored various types of information. When the server 140 is configured, the electronic device 130 that will be described below may perform an image analysis function of the in vitro diagnostic device 110 of the electronic device 130 while cooperating with the server 140, and/or the server 140 may perform all image analysis functions of the in vitro diagnostic device 110 and the electronic device 130 may receive information on the in vitro diagnosis result from the server 140.

According to various embodiments, the server 140 may include a server (an external server) of a medical institution, such as a hospital, a pharmacy, or an external institution, such as an insurance company. A distribution server, an analysis server, a storage server, and/or a learning server may be configured to communicate with the external server. The distribution server, the analysis server, the storage server, and/or the learning server may perform an operation of providing information on the in vitro diagnosis result to the electronic device 130, based on at least one piece of information that is received from the external server.

Meanwhile, according to various embodiments, without being limited to the illustration and/or description, the in vitro diagnostic system 1 may be configured to include more devices and/or mechanisms, or to include fewer devices and/or mechanisms. For example, referring to FIG. 1B, the in vitro diagnostic system 1 may not implement the holder 120 and/or the server 140. In this case, the electronic device 130 may perform an operation of analyzing an image of the in vitro diagnostic device 110 in the form of an on-device based on an application that is configured to provide an analysis service for the in vitro diagnostic device 110.

Hereinafter, an example of an in vitro diagnostic device 110 according to various embodiments will be described. As described above, for convenience of description, an in vitro diagnostic device 110 (an in vitro diagnostic device configured to diagnose Corona virus (COVID-19)) for an inspection of an immunochemical diagnosis method will be described as an example, but it is obvious to those skilled in the art that the various embodiments described below are not limited to the examples described and may be applied to the in vitro diagnostic device 110 that is configured for various types of diagnostic methods.

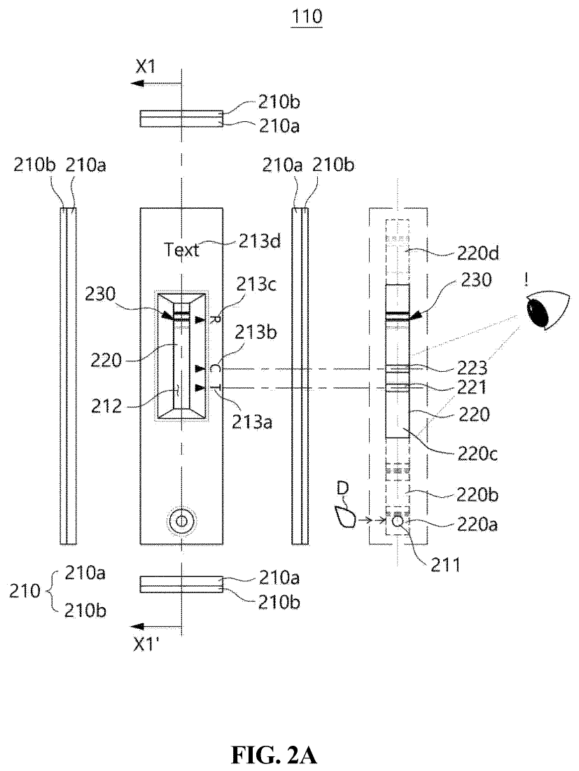

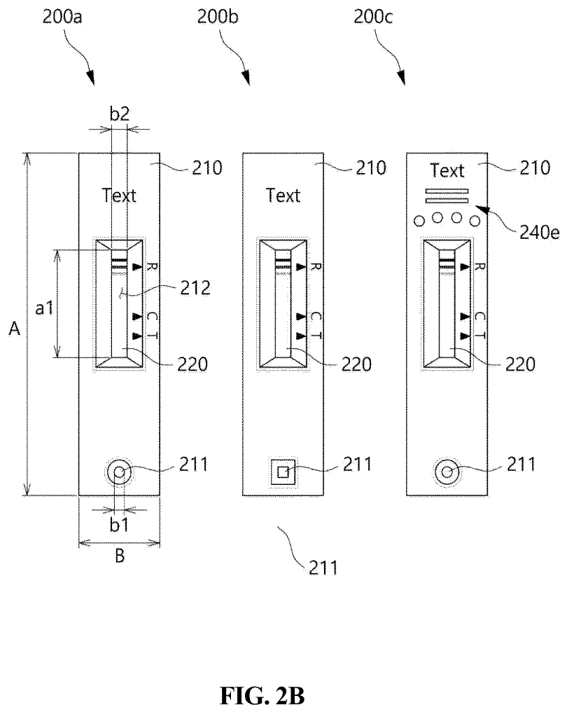

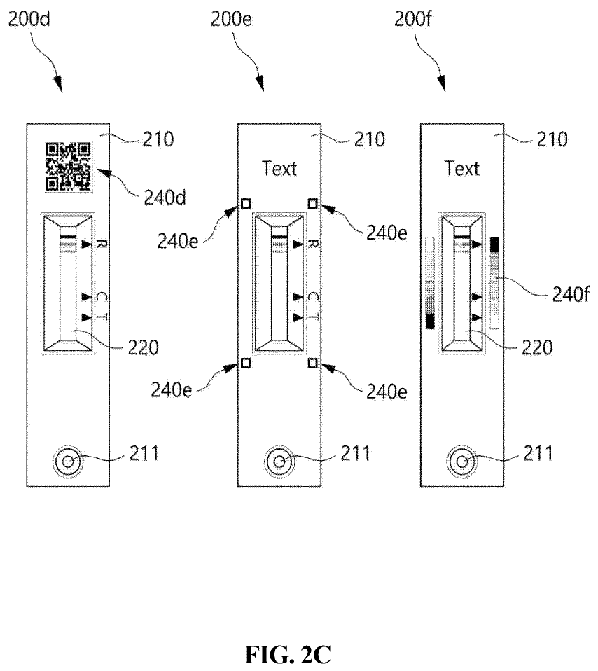

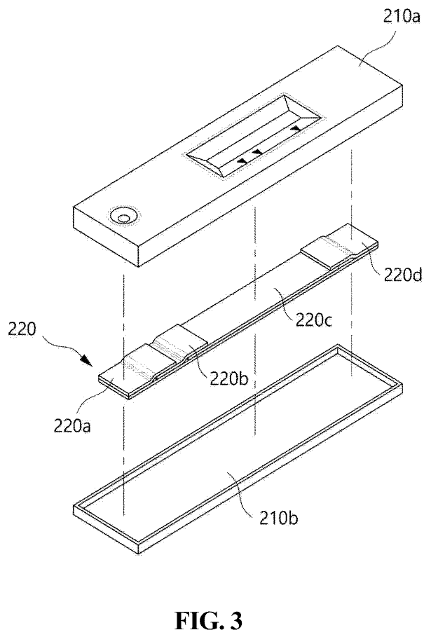

FIG. 2A is a view illustrating an example of the in vitro diagnostic device 110 according to various embodiments. Hereinafter, FIG. 2A will be further described with reference to FIGS. 2B, 2C, and 3.

FIG. 2B is a view illustrating various examples of a structure and/or configuration that constitutes an exterior of the in vitro diagnostic device according to various embodiments. FIG. 2C is a view illustrating examples of various types of visual markers that are formed in the housing 200 of the in vitro diagnostic device 110 according to various embodiments. FIG. 3 is an example of an exploded perspective view of the in vitro diagnostic device 110 according to various embodiments.

Referring to FIG. 2A, according to various embodiments, the in vitro diagnostic device 110 may include the housing 210 including a plurality of holes 211 and 212 and a pad (or a membrane) 220. Without being limited to the examples described above, the in vitro diagnostic device 110 may be configured to include more components or fewer components.

According to various embodiments, the housing 210 defines the exterior of the in vitro diagnostic device 110, and referring to 200a of FIG. 2B, it is obvious to those skilled in the art that the in vitro diagnostic device 110 may be designed with various exterior appearances while a length A and a width B of the housing 210 and a width B1 of the first hole b1 and a length A1 and a width B2 of the second hole 212 are variously formed.

Referring to FIG. 3, according to various embodiments, the housing 210 may include an upper housing 210a and a lower housing 210b that are coupled to each other, and the pad 220 may be configured to be disposed between the upper housing 210a and the lower housing 210b. Then, referring to FIG. 2A, a sample pad 220a for acquiring at least a portion (e.g., a specimen “D”), and a sample pad 220c including reaction areas 221 and 223 of the pad 220 may be exposed to the outside through a physical structure (e.g., the first hole 211 and the second hole 212) that is configured in the upper housing 210a.

According to various embodiments, the housing (e.g., the upper housing 210a and/or the lower housing 210b) may be configured to include at least one physical structure that forms the exterior.

For example, the upper housing 210a may include a physical structure that allows the specimen “D” to be delivered to the pad 220 (e.g., a sample pad 220a). As an example, referring to FIG. 2A, the upper housing 210a may include a first hole 211 having a specific (e.g., circular) shape for exposing a portion (e.g., a sample pad 220a) of the pad 220 so that the specimen “D” may be inserted into the pad 220. Without being limited to the illustrated and/or described examples, and referring to 200b of FIG. 2B, the shape of the first hole 211 may be formed in various types other than a circular shape, and although not illustrated, a physical structure (e.g., an introduction pipe, an introduction hole), in which the specimen “D” may be delivered to the pad 220, may be formed in a form, in which a portion (e.g., sample pad 220a) of the pad 220 is not exposed, instead of the first hole 211.

As another example, the upper housing 210a may include a second hole 212 for exposing the reaction areas 221 and 223 of the pad 220 so that the user may observe the in vitro diagnosis result that is expressed on the pad 220 (e.g., the sample pad 220c). As will be described later, at least a portion (e.g., the sample pad 220c including the reaction areas 221 and 223) exposed through the second hole 212 may be photographed by the electronic device 130.

As another example, the lower housing 210b may be configured to include a surface that is positioned on a bottom surface.

As another example, a physical structure, such as an inclined surface, an engraved (or concave) portion), an embossed (or protruding) portion, and/or a step, may be formed in at least a partial area of the housing 210. For example, referring to FIG. 2A, an upper housing 210a may be configured to include an inclined area around the first hole 211 and the second hole 212, an engaged/embossed area that surrounds the inclined area, and/or a stepped area. As another example, without being limited to the description and/or illustration, and referring to 200B of FIG. 2B, it is obvious to those skilled in the art that a unique physical structure for specifying (or identifying) a specific in vitro diagnostic device 110 may be designed, such as the shape of a physical structure (the inclined area/engraved area embossed area/stepped area) around the first hole 211 is formed in various types.

Meanwhile, referring to 200c of FIG. 2B, without being limited to examples of described and/or illustrated physical structures, the housing 210 (e.g., upper housing 210a) may be configured to include various kinds of physical structures 240c.

According to various embodiments, the housing 210 may be configured to include at least one visual marker. The at least one visual marker may include a text, a figure, and/or a code (e.g., a QR code, a bar code), and, without being limited to the examples described above, may further include various types of contents that are printed (or formed) on the housing 210 and may be recognizable by a user (or a camera of the electronic device 130).

For example, referring to FIG. 2A, an area near the second hole 210, through which the pad 220 of the upper housing 210a is exposed, may include texts (e.g., a “T” marker (or text) 213a, a “C” marker (or text) 213b, a sign marker 213c), and/or a figure for indicating the reaction areas 221 and 223 and/or signs 230 configured on the pad 220, and at least one additional text 213d. The “T” marker 213a indicates a reaction area 221 (hereinafter, a test area), in which the control line is expressed, the “C” marker 213b indicates a reaction area 223 (hereinafter, a control area), in which the control line is expressed, and the sign marker 213c may correspond to at least one sign marker 230.

As another example, referring to 200d of FIG. 2c, a code (e.g., a QR code) 240d may be configured on a partial area of the upper housing 210a. The QR code 240d may be configured to include various types of information (e.g., identification information of the in vitro diagnostic device 110, such as a product/serial number, information on a type of the product, information on a reaction time, LOT information, information on a quantitative value during a reaction, and the like) associated with the in vitro diagnostic device 110. Without being limited to the illustrated and/or the described examples, a code that is configured to provide specific information in addition to a QR code, such as a barcode, may be formed.

As another example, referring to 200e of FIG. 2C, at least one guide marker 240e may be configured on a partial area of the upper housing 210a. The electronic device 130 may be used to determine a disposition state (e.g., an inclination) (or a photographing state) of the in vitro diagnostic device 110 based on the at least one guide marker 240e (based on e.g., position and/or a shape), and/or to detect a position (e.g., a position of the pad 220) of at least a partial area of the in vitro diagnostic device 110 that may be identified by the guide marker 240e.

As another example, referring to 200f of FIG. 2C, a reference sign 240f may be configured on a partial area of the upper housing 210a. For example, as illustrated, the reference sign 240f may include a reference brightness signal (or a grayscale sign) for deriving a quantitative value of the reagent reaction, but without being limited to the illustrated example, may be configured to include various types of signs for correction. For example, the electronic device 130 may detect a brightness value corresponding to a reaction result (e.g., a test line or a control line) by using the reference sign 240f, and may identify a quantitative value of the reagent reaction corresponding to the detected brightness value based on pre-stored information on a quantitative value of the reagent reaction corresponding to the brightness value. The identified quantitative value may be used to correct a biometric information measurement value (e.g., a concentration of antibodies and/or antigens) and/or a color value of the reaction result (e.g., a test line or a control line).

The characteristics (e.g., a position of the physical structure, a shape of the physical structure) of the exterior configuration (e.g., a physical structure or a visual marker) of the in vitro diagnostic device 110 described above may be used as information for identifying the type and disposition state of the in vitro diagnostic device 110, and/or a position of a specific area (e.g., the pad 220, or the reaction areas 221 and 223). For example, information on the pre-configured exterior configuration for each in vitro diagnostic device 110 may be stored in the electronic device 130 in advance, and, the electronic device 130 may determine the type (e.g., information that indicates which in vitro diagnostic device 110 is for which company's in vitro diagnostic device 110) of in vitro diagnostic device 110, the disposition state and/or a position (a position of the pad 220) of the in vitro diagnostic device 110, based on the characteristics (e.g., the position of the “T” marker 213a, and the position of the pad 220) of a specific exterior configuration that is detected from the image of the in vitro diagnostic device 110 based on the pre-stored information. As an example, the electronic device 130 may identify the position of the pad 220 that is located in a specific direction and/or a specific distance of the in vitro diagnostic device 110, which are stored in advance from the corresponding marker based on the position of the “T” marker 213a and/or the position of the “C” marker 213b.

Meanwhile, although it is illustrated and/or described that the upper housing 210a and the lower housing 210b are coupled to each other, without being limited to the illustrated and/or described examples, the upper housing 210a and the lower housing 210b may be integrally formed.

According to various embodiments, the pad 220 may be configured to move the specimen “D” when the specimen “D” is introduced, add a specific inspection material to the specimen “D”, and visually express (e.g., express a test line) information (hereinafter, diagnosis result information) on presence of a disease based on the specimen “D” and sample material, to which the specific inspection material is added.

Referring to FIG. 3, according to various embodiments, the pad 220 may include a plurality of portions 220a, 220b, 220c, and 220d. For example, the pad 220 may be configured to include a sample pad 220a, a conjugate pad 220b, a sample pad 220c, and an absorption pad 220d, but without being limited to the described and/or illustrated examples, may be configured to include more pads or fewer pads.

For example, the sample pad 220a may be a pad for acquiring a specimen “D”.

For example, the conjugate pad above 220b may be configured to contain a substance (e.g., antibodies that are color-labeled and may be bind to antigens in the case of an antigen inspection method, and/or antigens that are color-labeled and may bind to antibodies in the case of an antibody inspection method) that specifically binds to an analysis target (e.g., an antigen in the case of an antigen inspection method, and/or an antibody in the case of an antibody inspection method) contained in specimen “D”.

For example, the sample pad 220c may include a reaction area (e.g. a reaction area 221, in which a test line is indicated) including a sample material that is configured to react with an analysis target contained in the specimen “D”, and a reaction area (e.g., a reaction area 223, in which a control line is expressed) including a sample material that is configured to react a material provided from the conjugate pad 220b.

For example, the absorption pad 220d may be a pad that is configured with a material having a high absorption property.

According to various embodiments, the pad 220 may be formed of a material that moves the specimen “D” in one direction according to a capillary phenomenon when the specimen “D” is introduced. Accordingly, when the antigen-based method, among immunochemical diagnostic methods, is described as an example, the specimen “D” may flow sideways along the sample pad 220a to be delivered to the conjugate pad 220b as illustrated in FIG. 2A. At least a portion of the material (e.g., an antigen) in the specimen “D” may bind to a material (e.g., a labeled antibody) in the conjugate pad 220b. At least a portion of the material in the combined specimen “D” and the material in the conjugate pad 220b (e.g., the labeled antibody) may flow laterally along the sample pad 220c in the direction of the absorption pad 220d, in the first reaction area (e.g., the test area 221), the labeled color may be expressed as a specific line (e.g., test line) as the material in the bound specimen “D” reacts with (e.g., binds to) the sample material and is arranged, and in the second reaction area (e.g., the control area 223), may be expressed as a specific line (e.g., a control line) as the material (e.g., the labeled antibody) in the conjugate pad 220b reacts with (binds to) the sample material and is arranged. Then, although it has been described that the color is expressed as a specific line on the pad 220, it is obvious to those skilled in the art that the color may be expressed in various forms, such as at least one point, a polygonal shape, a circle, which has a color other than a line shape. Accordingly, in the specification, the description and/or illustration in the form of the inspection line may be understood in the form of at least one point, a polygonal shape, circle, and the like that have the above-described color.

Meanwhile, an example of the in vitro diagnostic device 110 for performing an inspection based on an immunochemical diagnosis method has been described, but it may also be configured as an in vitro diagnostic device (110) for performing an inspection based on various other types of diagnosis methods.

According to various embodiments, the pad 220 (e.g., the sample pad 220b) may include at least one reaction area (e.g., the test area 221 and the control area 223) and at least one sign 230 described above. Meanwhile, without being limited to the examples described above, the pad 220 may be configured to include more components or to include fewer components. For example, the pad 220 may be configured to further include a material, a member, and/or a sign for indicating a reaction time from a time point, at which the specimen “D” is introduced, to the current time.

According to various embodiments, the at least one sign 230 may be configured to include at least one sign for correcting at least one piece of information (e.g., red (R), green (G), blue (B)), and/or a biometric information measurement value) that are detected from the pad 220 in the image of the in vitro diagnostic device 110.

According to various embodiments, when at least one sign 230 is configured in plural, the at least one sign 230 may include a plurality of signs having a specific color corresponding to each other (e.g., the same) and characteristic values (e.g., a brightness value and a chroma value) of different stages. The specific color may be a color that is associated with a reaction result (e.g., a test line and a control line) that is expressed on the reaction areas 221 and 223 of the pad 220. Based on the at least one sign 230, the color of the reaction result (e.g., a test line and a control line) that indicates the in vitro diagnosis result expressed in the reaction areas 221 and 223 of the pad 220 may be corrected. For example, the electronic device 130 may detect a photographing environment value (e.g., a brightness value) based on the at least one sign 230, and may use the detected photographing environment value to acquire the color value (e.g., red (R), green (G), blue (B)), and/or the biometric information measurement value of the reaction result of the in vitro diagnostic device 110. As a detailed example, the electronic device 130 may determine information (e.g., a tertiary regression function) for measuring the biometric information measurement value corresponding to the photographing environment value (e.g., a brightness value) (e.g., determine the coefficient of the function, and/or select a corresponding one of a plurality of functions itself), and may identify (e.g., based on the result values of inputting each of the R, G, and B values into the function) the biometric information measurement value (e.g., a concentration of an antigen or an antibody). Meanwhile, without being limited to the described example, the electronic device 130 may perform an operation of correcting the image of the in vitro diagnostic device 110 based on a visual attribute value (e.g., a color or a brightness) that is identified based on the at least one sign 230.

According to various embodiments, without being limited to the illustrated and/or described examples, and a color of the at least one sign 230 may be configured to be changed depending on a reaction with the specimen “D”, and may be configured in different colors. Furthermore, a grayscale sign may be configured instead of a sign having the color.

According to various embodiments, when the least one sign 230 is provided in plural, the signs 230 may be formed to be spaced apart from each other by a specific interval. However, without being limited to the description, and the signs 230 may be formed to contact each other, or may be formed to be spaced apart from each other by different intervals between the signs 230.

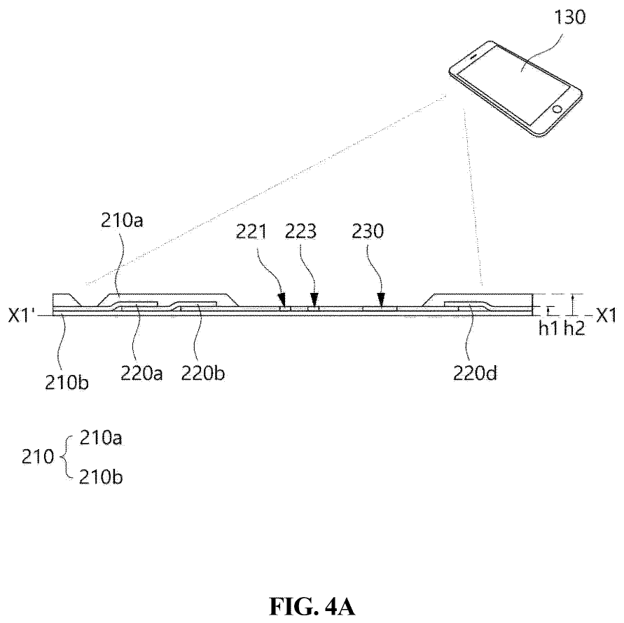

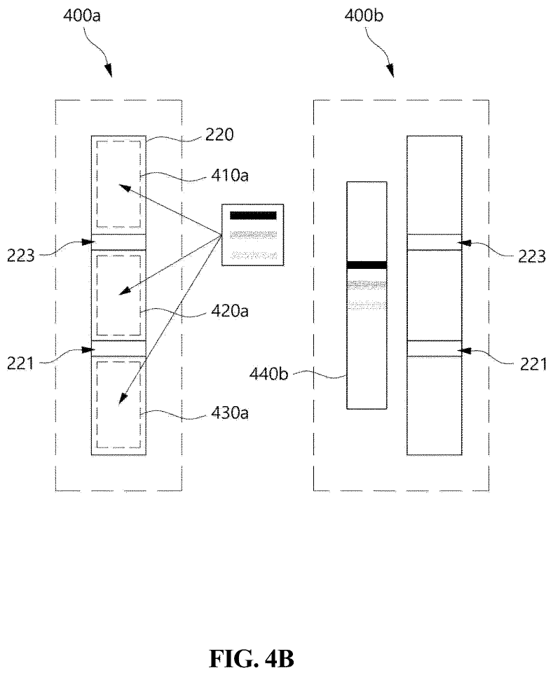

Hereinafter, referring to FIGS. 4A and 4B, an example of a position of at least one sign 230 according to various embodiments will be described. A repeated description of the description of FIGS. 2 to 3 will be omitted.

FIG. 4A is a cutting view for observing a side surface of the in vitro diagnostic device 110 with respect to X1-X1′ according to various embodiments. FIG. 4B is a view illustrating another example of a position of at least one sign 230 of the in vitro diagnostic device 110 according to various embodiments.

Referring to FIG. 4A, according to various embodiments, a step may be formed between the housing 210 (e.g., the upper housing 210a) and the pad 220. For example, a height h2 of the housing 210 (e.g., the upper housing 210a) and a height h1 of the pad 220 from a lower surface (e.g., the lower housing 210b) of the in vitro diagnostic device 110 may be different. Accordingly, in a state, in which the camera of the electronic device 130 is located to face the upper housing 210a, the photographing environment of the upper housing 210a and the photographing environment of the pad 220 may be different due to the step (or the different heights h1 and h2) when the in vitro diagnostic device w10 is photographed by the electronic device 130. As an example, the photographing environment may become different based on a difference between a photographing distance to the upper housing 210a and a photographing distance to the pad 220 from a camera of the electronic device 130. As another example, a contrast (e.g., presence of a shadow) between the housing 210a and the pad 220 may become different due to the step. Accordingly, when the sign 230 is configured on the housing 210, the accuracy of the sign 230 may deteriorate to be used to correct a diagnosis result sign on the reaction areas 221 and 223, on which it is displayed on pads having different photographing environments.

In consideration of the above difference of the photographing environments, according to various embodiments, the at least one sign 230 may be configured on the pad 220, and thus, the at least one sign 230 may be configured at the same height h1 as those of the reaction areas 221 and 223. Accordingly, the accuracy for correcting a diagnosis result sign on the reaction areas 221 and 223 may be improved based on the sign 230 that is identified from the captured image of the in vitro diagnostic device 110.

Referring to 400a of FIG. 4B, according to various embodiments, the at least one sign 230 may be formed in various areas 410a, 420a, and 430a on the sample pad that is divided (or classified) by the reaction areas 221 and 223. For example, it has been described that the sign 230 is formed in an upper end area 410a that is most distant from the first hole 211, into which the specimen “D” is introduced, but as illustrated in 400a of FIG. 4B, the sign 230 may be configured not only in the areas 420a and 430a between the reaction areas 221 and 223, but in a lower end area 430a that is closest to the first hole 211.

Referring to 400b of FIG. 4B, according to various embodiments, at least one sign 230 may be displayed on a portion 440b (e.g., a pad) that is formed separately from the pad 220, in which the reaction areas 221 and 223 are configured. Even in this case, the separately formed portion 440b (e.g., pad) may be disposed in an interior of the housing 210 such that the height of the pad 440b from the lower surface of the in vitro diagnostic device 110 corresponds to the height of the pad 220, in which the reaction areas 221 and 223 are configured. Then, the portion 440b may be configured of the same material as that of the pad 220.

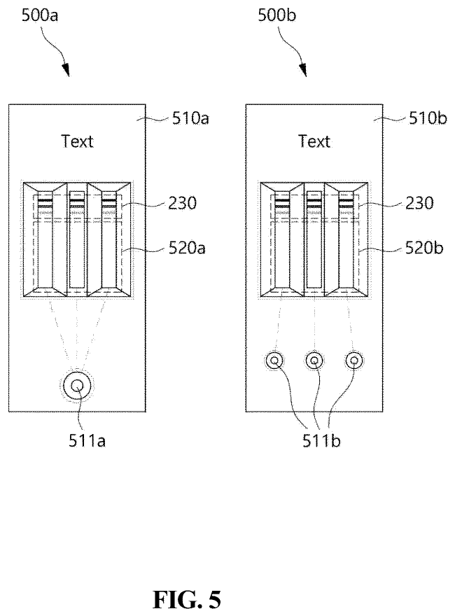

Hereinafter, an example of the in vitro diagnostic device 110 that is configured to include a plurality of pads according to various embodiments will be described with reference to FIG. 5. A repeated description of the description of FIGS. 2 to 4 will be omitted.

FIG. 5 is a view illustrating an example of the in vitro diagnostic device 110 that is configured to include the plurality of pads according to various embodiments.

Referring to 500a of FIG. 5, an in vitro diagnostic device 510a may be configured to include a single hole 511a and a plurality of pads 520a for introducing the specimen “D”. Then, the plurality of pads 520a may be configured to diagnose the same type of disease by acquiring the same specimen “D” that is introduced through a single hole 511a, but without being limited to the description, may be configured to diagnose different types of diseases. Then, each of the plurality of pads 520a is configured to include a sign 230, and, accordingly, when being photographed, the image of each of the pads may be corrected by using the sign 230 thereof. In this case, the plurality of pads 520a may be disposed in the interior of the housing 210 such that the heights of the plurality of pads 520a from the lower surface of the in vitro diagnostic device 110 correspond to each other.

Referring to 500b of FIG. 5, an in vitro diagnostic device 510b may be configured to include a plurality of holes 511b for introducing the specimen “D” and a plurality of pads 520b corresponding to the plurality of holes 511b. Then, the plurality of pads 520b may be configured to diagnose the same type of disease by acquiring the specimen “D” that are introduced through the different holes 511b, but without being limited to the description, may be configured to diagnose different types of diseases. Then, each of the plurality of pads 520b is configured to include a sign 230, and, accordingly, when being photographed, the image of each of the pads may be corrected by using the sign 230 thereof. In this case, the plurality of pads 520b may be disposed in the interior of the housing 210 such that the heights of the plurality of pads 520b from the lower surface of the in vitro diagnostic device 110 correspond to each other.

Hereinafter, an example of a holder 120 according to various embodiments will be described.

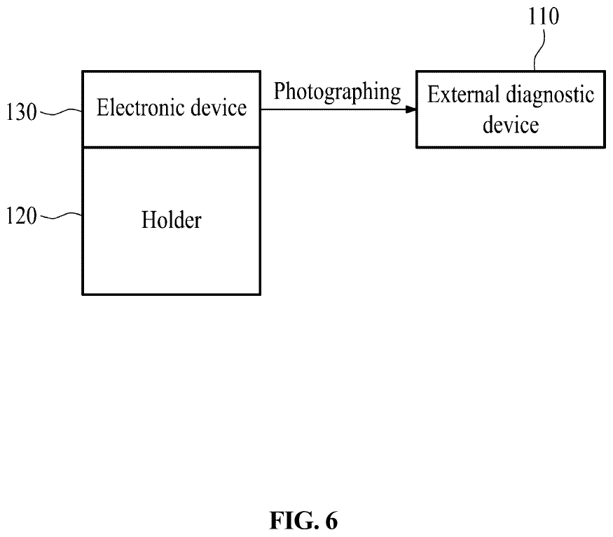

FIG. 6 is a view illustrating a use example of the in vitro diagnostic system 1 according to various embodiments.

Referring to FIG. 6, according to various embodiments, the user may photograph the in vitro diagnostic device 110 while physically holding the electronic device 130 in the holder 120. When the user photographs the in vitro diagnostic device 110 while gripping the electronic device 130, there may be difficulties in satisfying an appropriate photographing condition (e.g., a photographing inclination or a photographing distance) for photographing the in vitro diagnostic device 110 due to shaking of hands and the like. Accordingly, when the electronic device 130 is held, the holder 120 may be provided to a user who uses the in vitro diagnostic system 1 to satisfy an appropriate photographing condition (e.g., a photographing inclination or a photographing distance).

According to various embodiments, the holder 120 may include various types of holders. For example, the holder 120 may include a type of holder (hereinafter, a holder for a general user) for a general user who purchases the in vitro diagnostic device 110, and a type of holder (hereinafter, referred to as an expert holder) for an expert (e.g., medical manpower, such as a doctor and/or a nurse) who diagnoses a disease by using the in vitro diagnostic device 110 in a medical facility such as a hospital. As another example, the holder 120 may be configured for various types of holders for each purpose. For example, the holder 120 may be configured for each purpose, such as a disposable use and/or a multiple measurement use.

Hereinafter, an example of the holder 120 for a general user according to various embodiments will be described.

According to various embodiments, the holder 120 for a general user is a package (or a packaging container or packaging box) that is configured to receive the in vitro diagnostic device 110, and may be modified to provide a function as a holder for holding the electronic device 120. Meanwhile, without being limited to the described example, and the holder 120 for a general user may be a component that is provided together with another in vitro diagnostic device 110 in a package, other than a package.

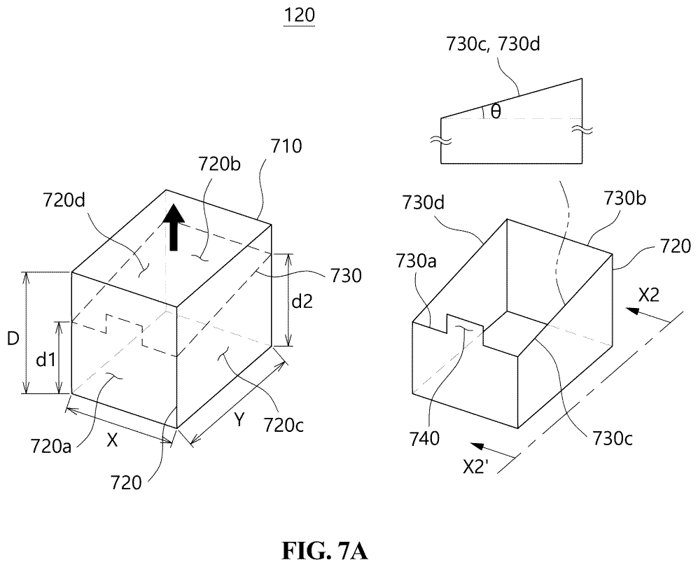

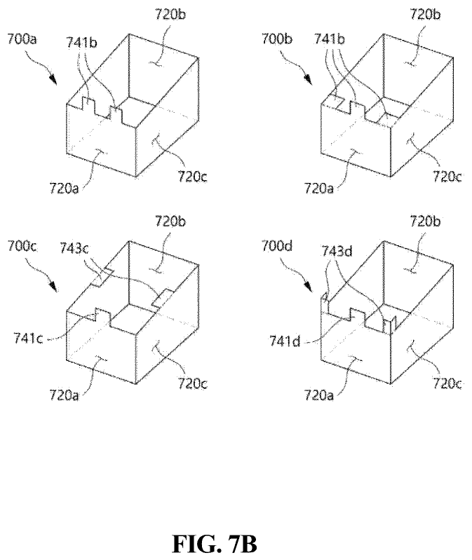

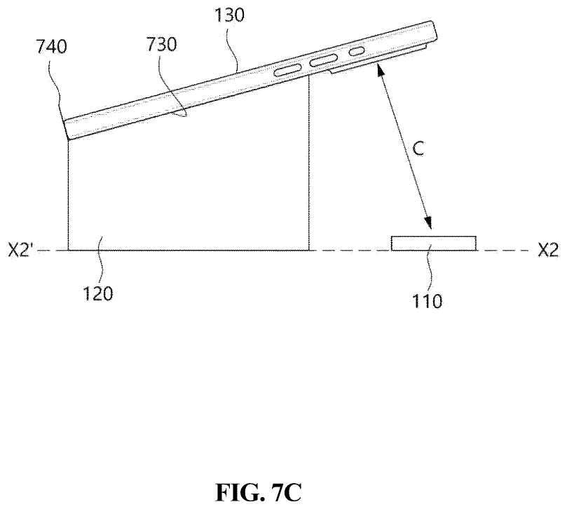

FIG. 7A is a view illustrating an example of the holder 120 according to various embodiments. Hereinafter, FIG. 7A will be described with reference to FIGS. 7B to 7C.

FIG. 7B is a view illustrating examples of a support part 740 that is formed in the holder 110 according to various embodiments. FIG. 7C is a view illustrating an example of a state, in which the electronic device 130 is held on the holder according to various embodiments.

Referring to FIG. 7A, according to various embodiments, the holder 120 (or a package) may include a body including an upper housing 710, a lower housing 720, and at least one perforation structure 730. The body may be configured in a rectangular parallelepiped shape that is designed to have a width X of 50 mm to 80 mm, a length Y of 80 mm to 120 mm, and a height D of 100 mm to 160 mm. For example, the body may be configured in a rectangular parallelepiped shape that is designed to have a width X of 65 mm, a length Y of 100 mm, and a height D of 130 mm. However, without being limited to examples of the described numerical values and/or shapes, and the holder 120 may be configured in various types of shapes (e.g., a cylindrical shape) that may be deformed to provide an area, in which the electronic device 130 may be held as the upper housing 710 and the lower housing 720 are separated. Meanwhile, without being limited to the described and/or illustrated examples, and the holder 120 may be configured to include more physical structures and/or visual markers.

According to various embodiments, the lower housing 720, from which the upper housing 710 is removed, may provide an area, in which the electronic device 130 may be held. In other words, the lower housing 720 may support the electronic device 130.

According to various embodiments, the at least one perforation structure 730 may be a structure, in which the body of the holder 120 is formed to be separated into the upper housing 710 and the lower housing 720. For example, the at least one perforation structure may be configured in the form of a perforation area and/or a perforation line.

According to various embodiments, the at least one perforation structure 730 is formed in a continuous (or connected) shape along the side surfaces 720a, 720b, 720c, and 720d of the body of the holder 120, and may be formed in different shapes for the side surfaces 720a, 720b, 720c, and 720d of the body.

For example, the perforation structures 730c and 730d of the side surfaces 720d and 720c in a lengthwise direction may be formed at a specific inclination to form a specified angle range (θ) with the lower surface of the holder 120. For example, the angle range (θ) may be in the range of 5 to 30 degrees. As another example, the angle range (θ) may be in the range of 10 to 20 degrees, and in this case, when the flash photographing is performed while the electronic device 130 is held on the holder 120, an influence of the light, which is caused by the flash, may be minimized. As a result, support areas that form a specific angle range with the lower surface to support at least a portion of the electronic device 120 may be formed at upper portions of the side surfaces 720d and 720c of the lower housing 720 in the lengthwise direction, from which the upper housing 710 is removed. Referring to FIG. 7C, the electronic device 120 may be held at an angle range of 10 to 20 degrees with the lower surface (or the ground) of the holder 120 by the support areas of the lower housing 720.

As another example, the perforation structures 730a of the side surfaces 720a and 720b, in which the height d1 is shorter, among the side surfaces 720a and 720b in the widthwise direction, may be formed to protrude upward between areas that are parallel to the lower surface of the holder 120, and the areas. Accordingly, a support area that may support at least a portion of the electronic device 120 may be formed on the side surface 720a of the lower housing 710, from which the upper housing 710 is removed, and a support part 740 having a protruding shape may be formed. The support part 740 may be configured of a material that is strong, which is from other parts of the holder 120, to support the electronic device 120, or may further include a member for supporting the electronic device 120, but is not limited to the examples described.

As another example, the perforation structure 730b of the side surface 720b having a longer height d2, among the side surfaces 720a and 720b in the widthwise direction, may be formed in a shape that is parallel to the lower surface of the holder 120.

Meanwhile, without being limited to the illustrated example, according to various embodiments, the support part 740 may be formed at at least a portion of the plurality of side surfaces 720a, 720b, 720c, and 720d of the body of the holder 120, and the perforation structure 730 may be formed therein.

For example, a plurality of support parts 740 may be formed. For example, referring to 700a of FIG. 7B, the perforation structure 730 may be formed to protrude upward from one side surface 720a a plurality of times such that a plurality of support parts 741a are formed on one side surface 720a. As another example, referring to 700b of FIG. 7B, a plurality of support parts 741b and 743b are formed, and the support part 741b may be formed at a middle portion of one side surface 720a, and a perforation structure 730 may be formed to form support parts 743b at portions that are adjacent to the other side surfaces 720c and 720d than the one side surface 720a. In this case, at least a portion 741b of a plurality of support parts 740 may be used to support the electronic device 130 in a protruding form and/or at least a portion 743b thereof may be used in a folded form, but is not limited thereto. A folding structure that facilitates folding may be further formed on the support part 743b that is used in the folded form.

As another example, the support part 740 may be formed for each of the plurality of side surfaces 720a, 720b, 720c, and 720d. As an example, referring to 700c of FIG. 7B, the support parts 741c and 743c may be formed on the side surfaces 720c and 720d in the lengthwise direction as well as on one side surface 720c and 720d having a low height in the widthwise direction. Meanwhile, without being limited to the illustrated and/or described examples, and a plurality of support parts may be formed for the side surfaces 720a, 720c, and 720d, respectively. Accordingly, the perforation structure of the side surfaces 720c and 720d in the lengthwise direction may also have a shape that protrudes upward. Then, at least a portion 741c may protrude, and the support parts 741c and 743c may be used in a folding shape on the side surfaces 720c and 720d in the lengthwise direction. A folding structure that facilitates folding may be further formed on the support part 743c that is used in the folded shape. For example, in an area, to which the side surfaces 720c and 720d connected to the used support part 743c are connected, a folding structure that facilitates folding in the direction of the interior space of the holder 120 may be formed.

As another example, the support part 740 may be formed to be connected to two or more side surfaces. As an example, referring to 700d of FIG. 7B, the support part 741d may be formed on one side surface 720a having a low height in the widthwise direction, and support parts 743d may be formed at a portion that connects the one side surface 720a having a low height in the widthwise direction to the side surfaces 720c and 720d in the length direction. Meanwhile, without being limited to the examples described, and the support part 740 may be formed in a form that connects three or more side surfaces. Accordingly, the perforation structure 730 may be configured to protrude upward from a portion that connects the side surfaces 720c and 720d.

According to various embodiments, the perforation structure 730 may be formed in a specific area range of the body. For example, the perforation structure 730 may be formed in an area of the side surfaces 720c and 720d corresponding to a range of 0.5 or more and less than 0.9 of a height from the lower surface of the body to the upper surface of the body. As another example, it may be formed in an area of the side surfaces 720c and 720d corresponding to a range of 0.6 or more and less than 0.8 of a height from the lower surface of the body to the upper surface of the body. As a detailed example, the perforation structure 730 may be formed at a point of about 0.61 of a height on one side surface 720c, and the perforation structure 730 may be formed at a point of about 0.82 of a height on the other side surface 720d. Accordingly, when the electronic device 130 is held on the lower housing 720, from which the upper housing 710 is removed by the perforation structure 730, an appropriate photographing distance “C” may be formed.

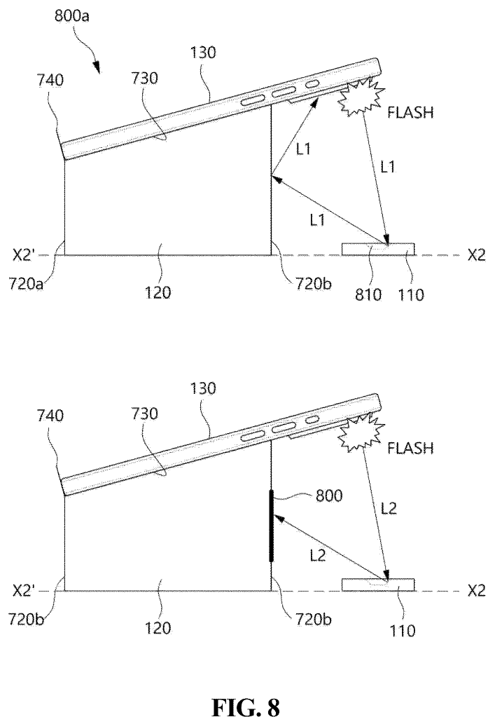

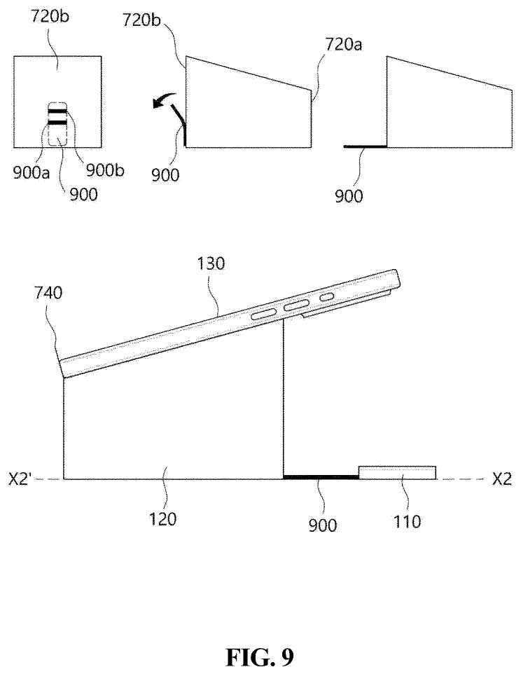

Hereinafter, referring to FIGS. 8 and 9, examples of physical structures that may be further configured in the holder 120 according to various embodiments will be described. A repeated description of the description in FIG. 7 will be omitted.

FIG. 8 is a view illustrating an example of the holder 120, in which an anti-reflection area 800 is formed, according to various embodiments. FIG. 9 is a view illustrating an example of the holder 120 including a member 900 for guiding the disposition of the in vitro diagnostic device 110 according to various embodiments.

According to various embodiments, referring to FIG. 8, at least a portion of the high-height side surface 720b of the lower housing 720, from which the upper housing 710 of the holder 120 is removed, may include an anti-reflection area 800 for preventing reflection of light. For example, the anti-reflection area 800 may be configured of a material for reducing or absorbing reflection of light, and/or may be configured of a material that transmits light, so that the light reflected from the in vitro diagnostic device 110 may not affect the electronic device 130. As an example, referring to 800a of FIG. 8, when photographing of the in vitro diagnostic device 110 is performed in a state, in which the electronic device 130 is held on the holder 120, flash light L1 that is generated by the electronic device 130 may be reflected by a unique structure 810 (e.g., an inclined area) of the in vitro diagnostic device 110. The reflected light L1 may be input to the side surface 720b of the holder 120, which is adjacent to the area, in which the in vitro diagnostic device 110 is disposed, may be reflected from the side surface 720b, and may be input to the electronic device 130, so that the photographed image may be affected. When the anti-reflection area 800 is formed on the corresponding side surface 720b, flash light L2 that is generated by the electronic device 130 is reflected by the in vitro diagnostic device 110 and is input to the side surface 720b, but reflection to the electronic device 130 may be prevented by the anti-reflection area 800. Accordingly, the accuracy of the image of the in vitro diagnostic device 110 may be improved.

According to various embodiments, the anti-reflection area 800 may be formed in an area that is spaced apart from the lower surface and/or the upper surface of the lower housing 720 by a specific distance. For example, the anti-reflection area 800 may be formed on a long-height side surface 720b while being spaced apart from the lower surface of the lower housing 720 by a distance that is greater than the height of the in vitro diagnostic device 110.

According to various embodiments, referring to FIG. 9, at least a portion of the long-height side surface 720b of the lower housing 720 of the body of the holder 120 may be configured to include a guide member 900 for guiding the disposition of the in vitro diagnostic device 110. For example, the guide member 900 may be formed by the perforation structure that is cut to enable the guide member 900 to protrude, and a distance, at which the in vitro diagnostic device 110 is disposed, may be guided by the length of the detached guide member 900. Accordingly, the user may recognize the in vitro diagnostic device 110 such that the in vitro diagnostic device 100 is disposed in an appropriate position more easily. Then, the guide member 900 may be configured to include identification lines 900a and 900b for guiding the electronic device 130 of the user for the sizes of the electronic device 130. For example, when the size of the electronic device 130 is of a relatively small type, the user may dispose the in vitro diagnostic device 110 in a position corresponding to the first identification line 900a, and when the size of the electronic device 130 is of a relatively large type, the user may dispose the in vitro diagnostic device 110 in a position corresponding to the second identification line 900b.

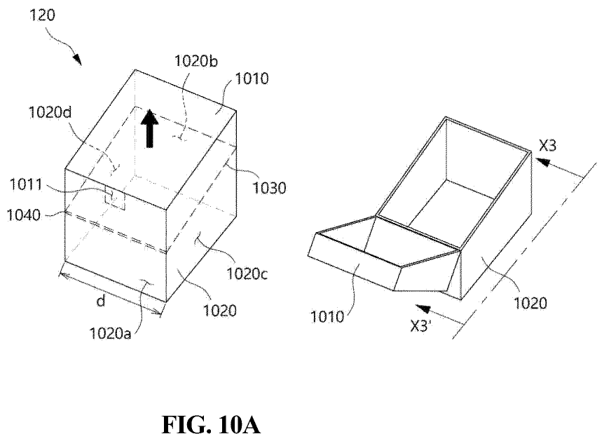

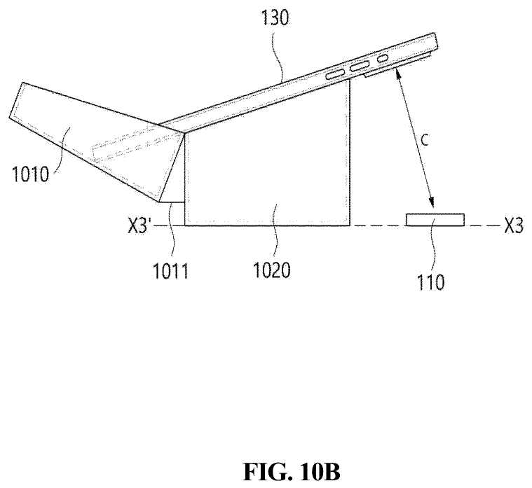

Hereinafter, referring to FIGS. 10A and 10B, examples of physical structures that may be further configured in the holder 120 according to various embodiments will be described. A repeated description of the description of FIGS. 7 to 9 will be omitted.

FIG. 10A is a view illustrating an example of the holder 120, in which the upper housing 1010 and the lower housing 1020 are configured to be connected to each other at at least a portion according to various embodiments. FIG. 10B is a view illustrating an example held on the electronic device 130 by the upper housing 1010 according to various embodiments.

Referring to FIG. 10A, according to various embodiments, the holder 120 may be configured to include an upper housing 1010, a lower housing 1020, and a disconnection structure 1030. Meanwhile, without being limited to the described and/or illustrated examples, and the holder 120 may be configured to include more physical structures and/or visual markers.

According to various embodiments, the disconnection structure 1030 may be configured to be connected to a plurality of side surfaces, and may be formed in different structures for the side surfaces 1020a, 1020b, 1020c, and 1020d, respectively. For example, the disconnection structure may be formed as a perforation structure for cutting the upper housing 1010 and the lower housing 1020, and may be formed as a connection structure 1040 for connection between the upper housing 1010 and the lower housing 1020 in another side surface. For example, referring to FIG. 10A the connection structure 1040 may be formed on a side surface 1020a in a widthwise direction, in which a height is relatively low, and the perforation structure 1030 may be formed on the other side surfaces 1020b, 1020c, and 1020d. The connection structure may be configured as a structure for easily folding in a direction of the one side surface 1020a. Accordingly, even when the upper housing 1020 is cut in the perforation structure 1030, the upper housing 1020 may be connected to a side surface 1020a in a widthwise direction, in which the height is low, and may be rotated (turned over) in a direction of one side surface 1020a. Accordingly, referring to FIG. 10B, when the electronic device 130 is held on the holder 120, while being held by an upper portion of the side surface 1020b in a widthwise direction, in which the height is high, the electronic device 130 may be supported by an inner surface of the upper housing 1020 rotated in a direction of the side surface 1020a in a widthwise direction, in which the height is low. For example, the electronic device 130 may be supported by an inner surface of an upper portion of the upper housing 1020 and/or an inner surface of a side portion of the upper housing 1020, in which the connection structure 1040 is formed.

According to various embodiments, the holder 120 may be configured such that the position of the upper housing 110 that is turned over is fixed. For example, referring to FIG. 10B, a stopper member 1011 that is formed in a partial area of the upper housing 1020 may protrude to contact a portion (e.g., the lower housing 1010), and thus a position of the upper housing 1020 may be fixed. For example, the stopper member 1011 may be configured to be cut off from an outer surface of a side portion of the upper housing 1020, at which the connection structure 1040 is formed, and protrude by a specific length. Further, without being limited to the described and/or illustrated examples, the stopper member 10111 may contact another portion (e.g., the ground) to fix a position of the upper housing 1020. As another example, the connection structure 1040 may be designed in a structure, in which a position of the upper housing 110 is fixed (e.g., no longer rotated) when a rotation angle thereof reaches a preset angle.

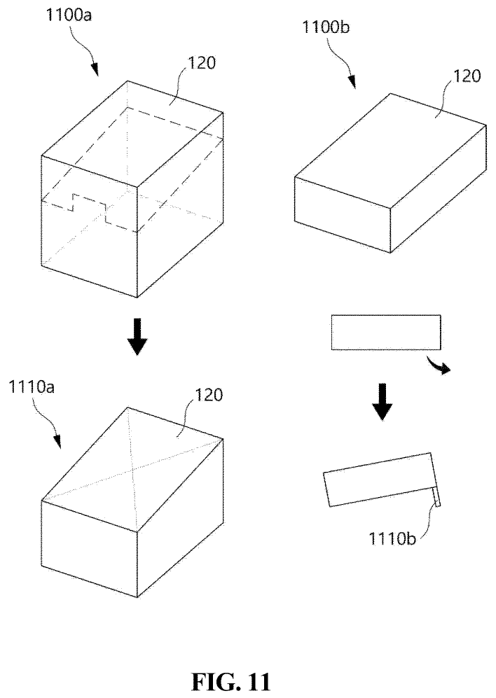

Hereinafter, referring to FIG. 11, examples in the form of the holder 120, in which the electronic device 130 may be held in the range of 10 to 20 degrees, will be described.

FIG. 11 is a view illustrating an example of the holder according to various embodiments.

Referring to FIG. 1100a, according to various embodiments, the bottom surface of the holder 120 may be used to hold the electronic device 130. In this case, a material (e.g., rubber, an adhesive member, or the like) for preventing the electronic device 130 from slipping may be further provided on a bottom surface 1110a of the holder 120 (e.g., the lower housing).

Referring to 1100b of FIG. 11, according to various embodiments, the holder 120 may be configured such that a protruding portion 1110b is formed on some side surfaces thereof. The protruding portion 1110b may be configured such that the protruding portion 1110b may be held to have a height in a range of 10 degrees to 20 degrees when the electronic device 130 is disposed (or held) on the other side surface facing the holder.

Meanwhile, without being limited to the illustrated and/or described examples, the holder 120, in which the electronic device 130 may be held in the range of 10 to 20 degrees, may be further configured.

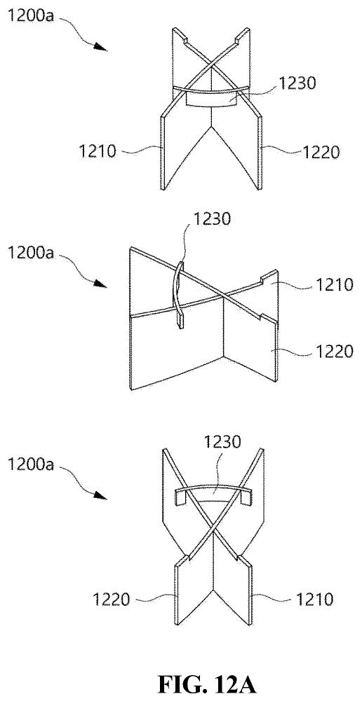

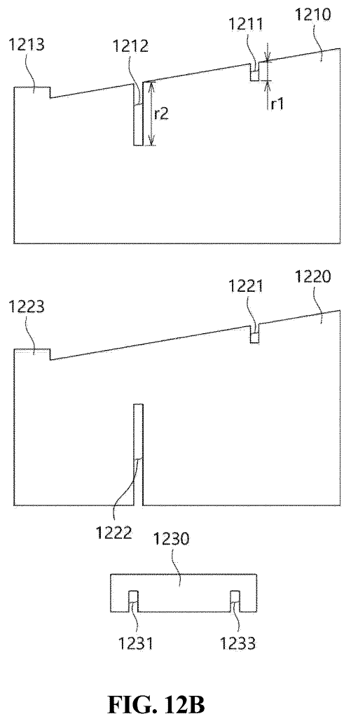

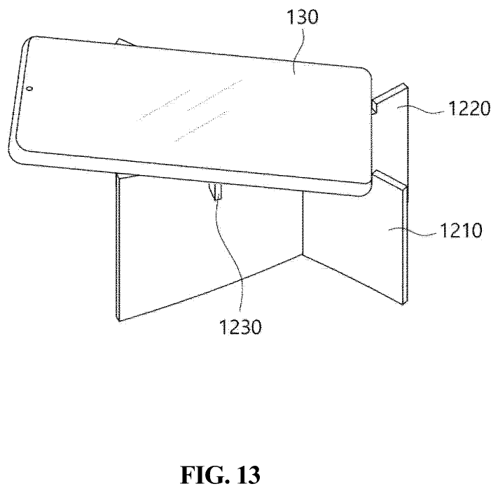

Hereinafter, referring to FIGS. 12 and 13, an example of the expert holder 120 according to various embodiments will be described.

FIG. 12A is a view illustrating examples of observing the expert holder 120 from various directions according to various embodiments. FIG. 12B is a view illustrating an example of components of the expert holder 120 according to various embodiments. Hereinafter, referring to FIG. 13, FIG. 12A and FIG. 12B will be described.

FIG. 13 is a view illustrating an example of the electronic device 130 that is held on the expert holder 120 according to various embodiments.

Referring to FIG. 12A, the expert holder 120 may be used to provide an appropriate photographing condition for photographing the in vitro diagnostic device 110 by holding the electronic device 130, like the general user holder 120 described in FIGS. 7 to 11. However, the expert holder 120 may be configured of a more rigid material compared to the holder 120 for a general user. For example, the holder 120 for a general user may be configured of a paper material, but the expert holder 120 may be configured of a plastic material. Accordingly, when the expert holder 120 is used in a specialized institution, such as a hospital, the expert holder 120 may be used a plurality of times.

Referring to FIG. 12B, the expert holder 120 may include a plurality of components. For example, the expert holder 120 may include a plurality of plates 1210, 1220, and 1230 that are configured to include different physical structures.

According to various embodiments, the first plate 1210 may include a lower portion, and an upper portion having an angle range of 10 to 20 degrees upward, compared to the lower portion. A support part 1213 that protrudes to support the electronic device 130 may be formed in an area corresponding to the lowest portion of the first plate 1210 at the upper portion.

According to various embodiments, the first plate 1210 may include a plurality of grooves 1211 and 1212. For example, the upper portion of the first plate 1210 may include grooves 1211 and 1212 that are formed in the direction of the lower portion. The grooves 1211 and 1212 may be configured for different purposes. For example, the first groove 1211 may be formed for the purpose of being coupled to the grooves 1231 and 1232 of the third plate 1230, which will be described later, and thus, may be formed to have a depth that is associated with the third plate 1230. For example, the first groove 1211 may be formed to have a depth corresponding to the length of the body of the third plate 1230, except for the depth of the grooves 1231 and 1232 of the third plate 1230. Accordingly, when the third plate 1230 is coupled to the first groove 1211, the upper surface of the third plate 1230 and the upper surface of the first plate 1230 may form the same surface, so that the electronic device 130 may be supported by the formed surface. As another example, the second groove 1212 may be formed for the purpose of being coupled to the fourth groove 1222 of the second plate 1220, which will be described later. Meanwhile, the depth of the first groove 1211 and the depth of the second groove 1212 may be formed to have different depths, but may be formed to have depths corresponding to each other, without being limited to description and/or illustration.

According to various embodiments, the second plate 1220 may also include a lower portion, such as the first plate 1210, and an upper portion, in which an angle range is formed in a range of 10 to 20 degrees upward, compared to the lower portion, and a support part 1223 may be formed.

According to various embodiments, the second plate 1220 may also include a plurality of grooves 1221 and 1223 like the first plate 1210. The third groove 1221 of the second plate 1220 may be formed in a position corresponding to a position, in which the first groove 1211 of the first plate 1210 described above is formed, and the third groove 1211 may be formed together with the first groove 1211, and thus, a repeated description thereof will be omitted. The fourth groove 1222 of the second plate 1220 may be formed in a position corresponding to a position, in which the second groove 1212 of the first plate 1210 is formed, and may be formed from a lower portion of the second plate 1220 in the direction an upper portion thereof. Accordingly, the second groove 1212 and the fourth groove 1222 are coupled to each other, so that the first plate 1210 and the second plate 1220 may be coupled to each other in a form of crossing each other.

According to various embodiments, a sum of the depths of the grooves 1212 and 1222 for coupling the first plate 1210 and the second plate 1220 to each other may be configured to be greater than the respective heights of the first plate 1210 and the second plate 1220 in positions, in which the grooves 1212 and 1222 are formed. For example, the depth of the second groove 1212 of the first plate 1210 may be a first depth (e.g., 32.5 cm), and the depth of the fourth groove 1222 of the second plate 1220 may be a second depth (e.g., 55 cm), whereas the height of each of the first plate 1210 and the second plate 1220 in positions, in which the grooves 1212 and 1222 are formed, may be a third height (e.g., 85.5 cm). Accordingly, in the state, in which the first plate 1210 and the second plate 1220 are coupled to each other, a marginal space may be formed between the grooves 1212 and 1222, and a degree of freedom of movement may be given when the first plate 1210 and the second plate 1220 are coupled to each other by the formed marginal space. However, without being limited to the description, and the sum of the depths of the grooves 1212 and 1222 of the first plate 1210 and the second plate 1220 may be configured to correspond to the heights of the first plate 1210 and the second plate 1220.

According to various embodiments, the third plate 1230 may include a lower portion, and an upper portion that is formed parallel to the lower portion. The plurality of grooves 1231 and 1232 that formed upward may be formed at lower portions of the third plate 1230, and the plurality of grooves 1231 and 1232 may be coupled to the first groove 1211 of the first plate 1210 and the third groove 1221 of the second plate 1220 described above, respectively. The plurality of grooves 1231, 1232, the first groove 1211 and the third groove 1221 may correspond to each other (e.g., 10 cm). Accordingly, referring to FIG. 13, when the first plate 1210, the second plate 1220, and the third plate 1230 are coupled to each other, upper surfaces of the first plate 1210, the second plate 1220, and the third plate 1230 may be formed so that there is no step, so that a surface, on which the electronic device 130 may be held, may be formed in the expert holder 120.

Meanwhile, without being limited to the description and/or illustration, and the plurality of components 1210, 1220, and 1230 of the expert holder 120 may be provided in a single coupled form.

Hereinafter, examples of components of the electronic device 130 according to various embodiments will be described.

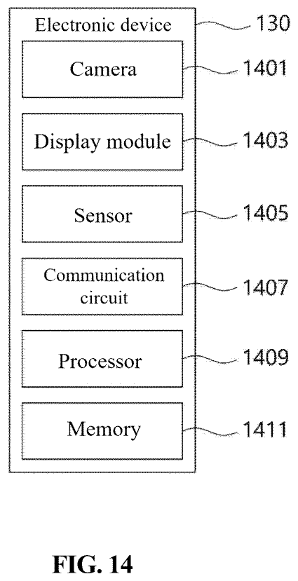

FIG. 14 is a view illustrating an example of components of the electronic device 130 according to various embodiments.

Referring to FIG. 14, according to various embodiments, the electronic device 130 may include a camera 1401, a display module 1403, a sensor 1405, a communication circuit 1407, a processor 1409, and a memory 1411. Without being limited to the description and/or illustration, the electronic device 130 may be configured to include more or fewer components.

According to various embodiments, the camera 1401 may capture a still image and a moving image. According to an embodiment, the camera 1401 may include one or more lenses, image sensors, image sign processors, or flashes.

According to various embodiments, the display module 1403 may visually provide information to the outside (e.g., the user) of the electronic device 130. The display module 1403 may include, for example, a display, a hologram device, or a projector, and a control circuit for controlling the corresponding device. According to an embodiment, the display module 1403 may include a touch sensor that is set to detect a touch, or a pressure sensor that is set to measure a magnitude of a force that is generated by the touch.

According to various embodiments, the sensor 1405 may detect an operation state (e.g., electric power or temperature) or an external environmental state (e.g., a user state) of the electronic device 130, and generate an electrical signal or data value corresponding to the sensed state. According to an embodiment, the sensor 1405 may include, for example, an inertia sensor, a inclination sensor, a gesture sensor, a gyro sensor, a pressure sensor, a magnetic sensor, an acceleration sensor, a grip sensor, a proximity sensor, a color sensor, an infrared sensor (IR) sensor, a biometric sensor, a temperature sensor, a humidity sensor, or a luminance sensor.

According to various embodiments, the communication circuit 1407 may support establishment of a direct (e.g., wired) communication channel or wireless communication channel between the electronic device 130 and an external electronic device (e.g., the server 140 of FIG. 1) and performance of a communication through the established communication channel. The communication circuit 1407 may include one or more communication processors that is operated independently of the processor 1409 (e.g., an application processor) and supports a direct (e.g., wired) communication or wireless communication. According to an embodiment, the communication circuit 1407 may include a wireless communication module (e.g., a cellular communication module, a short-range wireless communication module, or a global navigation satellite system (GNSS) communication module) or a wired communication module (e.g., a local area network (LAN) communication module or a power line communication module). A corresponding one of these communication modules may communicate with an external electronic device (e.g., server 140) through a first network (e.g., a short-distance communication network, such as Bluetooth, or wireless fidelity (Wi-Fi) direct or infrared data association (IrDA)) or a second network (a long distance communication network, a legacy cellular network, a 5G network, a next-generation communication network, the Internet, or a computer network (e.g., LAN or WAN). These various types of communication modules may be integrated into a single component (e.g., a single chip), or may be configured as a plurality of separate components (e.g., a plurality of chips). The wireless communication module may identify or authenticate the electronic device 130 in a communication network, such as the first network or the second network, by using subscriber information (e.g., an international mobile subscriber identifier (IMSI)) stored in the subscriber identification module. The wireless communication module may support 5G networks and next-generation communication technologies after the 4G network, for example, the NR access technology. The NR access technology may support high-capacity data enhanced mobile broadband (eMBB), terminal power minimization and massive machine type communications (mMTC), or ultra-reliable and low-latency communications (URLLC). For example, wireless communication modules may support high-frequency bands (e.g., mmWave bands) to achieve high data transmission rates. Wireless communication modules may support various technologies for securing performance in high-frequency bands, such as beamforming, massive array-introduced and multiple-output (MIMO), full dimensional MIMO (FD-MIMO), array antennas, analog beam-forming, or large scale antennas. The wireless communication module may support various requirements that are specified in the electronic device 130, an external electronic device (e.g., the server 140), or a network system (e.g., the second network 199). According to an embodiment, the wireless communication module may support a peak data rate (e.g., 20 Gbps or more) for realizing eMBB, a loss coverage (e.g., 164 dB or less) for realizing mMTC, or a U-plane latency (e.g., less than or equal to 0.5 ms, or less than or equal to a round trip of 1 ms, respectively) for realizing URLLC.

According to various embodiments, the processor 1409 may include at least one processor, in which at least some thereof are configured to provide different functions. For example, at least one other component (e.g., a hardware, or software component) of the electronic device 130 connected to the processor 1409 may be controlled by executing software (e.g., a program), and various data processing or operations may be performed. According to an embodiment, as at least a part of data processing or an operation, the processor 1409 may store a command or data that are received from another component (e.g., the sensor 1405, or the communication circuit 1407) in the memory 1411 (e.g., volatile memory), process a command or data that are stored in a volatile memory, and store the result data in a nonvolatile memory. According to an embodiment, the processor 1409 may include a main processor (e.g., a central processing unit or an application processor) or an auxiliary processor (e.g., a graphic processing unit, a neural processing unit (NPU), an image signal processor, a sensor hub processor, or a communication processor). For example, when the electronic device 130 includes a main processor and an auxiliary processor, the auxiliary processor may use lower electric power than the main processor or may be set to be specialized for a specified function. The auxiliary processor may be configured separately from the main processor or as a part thereof. The auxiliary processor may, for example, control at least some of functions or states that are related to at least one component (e.g., the display module 1403, the sensor 1405, the or communication circuit 1407), among the components of the electronic device 130, instead of the main processor together with the main processor while the main processor is in an active (e.g., application execution) state. According to an embodiment, the auxiliary processor (e.g., an image sign processor or a communication processor) may be configured as a part of another functionally related component (e.g., the camera 1401 or the communication circuit 1407). According to an embodiment, the auxiliary processor (e.g., a neural network processing device) may include a hardware structure that is specialized for processing an artificial intelligence model. The artificial intelligence model may be generated through machine learning. The learning may be performed, for example, in the electronic device 130 itself, in which the artificial intelligence model is performed, or may be performed through a separate server (e.g., the server 140). The learning algorithm may include, for example, supervised 1 learning, unsupervised learning, semi-supervised learning, or reinforcement learning, but is not limited to the above-described examples. The artificial intelligence model may include a plurality of artificial neural network layers.

The artificial neural network may include a deep neural network (DNN), a convolutional neural network (CNN), a recurrent neural network (RNN), a restricted Boltzmann machine (RBM), a deep belief network (DBN), a bidirectional recurrent deep natural network (BRDNN), a deep Q-network, or a combination of two or more of the above, but is not limited to the above-described examples. In addition to the hardware structure, the artificial intelligence model may additionally or alternatively include a software structure. For example, the artificial intelligence model may include an artificial intelligence model that is configured to provide diagnosis result information by analyzing an image of the in vitro diagnostic device 110. The diagnosis result information may be an artificial intelligence model that is configured to provide health information (or wellness information), such as a possibility of being affected by a disease, as well as the presence of a disease.

According to various embodiments, the memory 1411 may store various data used by at least one component (e.g., a processor 1409, or a sensor 1405) of electronic device 130. The data may include, for example, software (e.g., a program), and input data or output data for a related command. The memory 1411 may include a volatile memory or a nonvolatile memory. The memory 1411 may be configured to store an operating system, middleware or an application, and/or the artificial intelligence model described above.

In addition to the above-described components, the electronic device 130 may include an input module, a sound output module, an audio module, an interface, a connection terminal, a haptic module, a power management module, a battery, or an antenna module.

According to various embodiments, an operation of the electronic device 130 that will be described below may be performed based on execution of an application that is configured to provide an in vitro diagnosis result based on an image of the in vitro diagnostic device 110, which is stored (or installed) in the electronic device 130. Unless otherwise mentioned, the operation of the electronic device 130 described below may be performed in the server 140, and/or an operation of the server 140 may be performed in the electronic device 130.

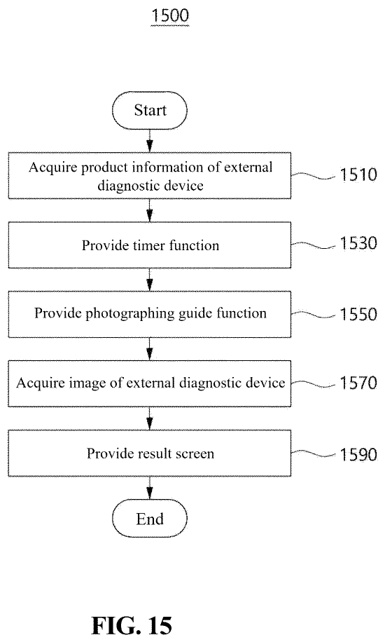

FIG. 15 is a flowchart 1500 illustrating operations based on various functions that are provided in a process of providing a diagnosis result by using an image of the in vitro diagnostic device 110, by the electronic device 130 (e.g., a processor 1409) according to various embodiments. According to various embodiments, the operations illustrated in FIG. 15 may be performed in various orders without being limited to the order illustrated. Furthermore, according to various embodiments, more operation than the operations illustrated in FIG. 15 or at least one operation that is less than the operations may be performed.

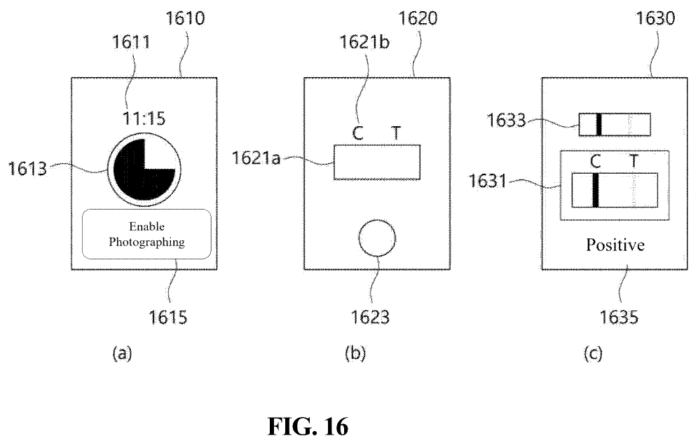

FIG. 16 is a view illustrating various functions that are provided by the electronic device 130 (e.g., the processor 1409) according to various embodiments.

According to various embodiments, the electronic device 130 may acquire product information of the in vitro diagnostic device 110 in operation 1510. For example, product information of the in vitro diagnostic device (110) may include identification information of the in vitro diagnostic device (110), such as a product/serial number of the in vitro diagnostic device 110, information on a reaction time (e.g., a minimum time (hereinafter, a first reaction time), by which the result of the reaction between the specimen “D”, to which a specific inspection material is added, and a sample material of the sample pad 220c may be relied upon (or the accuracy, such as a diagnostic sensitivity, becomes a threshold value or higher) after the specimen “D” is introduced, and/or a maximum time (hereinafter, a second reaction time), by which the result of the reaction between the specimen “D”, to which a specific inspection material substance is added, and the sample material of the sample pad 220c may be relied upon (or the accuracy, such as a diagnostic sensitivity, becomes a threshold value or higher) after the specimen “D” is introduced) for providing diagnostic result information, information on a photographing allowable time (e.g., a minimum time (hereinafter, a first photographing allowable time) for allowing photographing and/or a maximum photographing allowable time (hereinafter, a second photographing allowable time) for allowing photographing), information on the type of the in vitro diagnostic device 110, information on various components (e.g., the pad 220 of FIG. 2A) that constitute the in vitro diagnostic device 110, the reaction area (e.g., the test area 221 and the control area 223 of FIG. 2A), surrounding objects (e.g., a specific pattern and/or engraving), and/or product information, such as information on at least one guide object, which is used to provide the diagnostic result. Meanwhile, the above-described information is exemplary, and the electronic device 130 may acquire various product information that is used to provide a diagnosis result based on the identification information. According to an embodiment, the electronic device 130 may acquire at least a part of the remaining product information based on identification information of the in vitro diagnostic device 110. According to an embodiment, the electronic device 130 may acquire the above-described product information in parallel.

According to various embodiments, the electronic device 130 may acquire product information of the in vitro diagnostic device 110 through various methods. For example, the electronic device 130 may acquire an image of the in vitro diagnostic device 110 or the package (e.g., the holder 120 of FIG. 7A) of the in vitro diagnostic device 110 by using the camera 1401, and acquire the product information of the in vitro diagnostic device 110 based on the acquired image. As an example, a visual mark (e.g., QR code) that is configured to provide product information may be configured outside the in vitro diagnostic device 110 and/or the package (the holder 120 of FIG. 7A). The electronic device 130 may acquire product information based on a code (e.g., a QR code) that is identified from the image. As another example, the electronic device 130 may acquire at least a part of the above-described product information by receiving it directly from the user of the electronic device 130.

According to various embodiments, the electronic device 130 may provide a timer function in operation 1530. According to various embodiments, the electronic device 130 may set at least one timer time based on the acquired product information. For example, the electronic device 130 may set the first photographing allowable time (or the first reaction time) as a first timer time. For example, the electronic device 130 may set the second photographing allowable time (or the second reaction time) as a second timer time. According to various embodiments, the electronic device 130 may display a screen (hereinafter, a timer screen) for providing a timer function. According to various embodiments, information on a remaining time up to a specific time (e.g., the first photographing allowable time and/or the second photographing allowable time), and information and/or an object that is associated with the photographing function may be provided through the timer screen.

Referring to FIG. 16(a), the timer screen 1610 may include objects 1611 and 1613 that indicate a remaining time (hereinafter, a first remaining time) up to the first timer time (or, the first photographing allowable time (i.e., a minimum time, for which photographing is allowed)) and an object 1615 that is associated with the photographing function. Although not illustrated, the timer screen 1610 may include a separate object (not illustrated) that indicates a remaining time (hereinafter, a second remaining time) up to the second timer time (or the second photographing allowable time) (i.e., a time, at which the photographing starts to be restricted (or a maximum time, for which photographing is allowed)) or may also indicate the second photographing allowable time together through the object 1611 and/or the object 1613.

According to various embodiments, the remaining time until the first timer time may be displayed in the form of a text (e.g., “11:15”), such as the object 1611, and/or in the form of a graphic object (e.g., a circle and/or a fan shape), such as the object 1613.

According to various embodiments, photographing of the in vitro diagnostic device 110 may be allowed after the first timer time elapses. Meanwhile, photographing of the in vitro diagnostic device 110 may not be allowed after the second timer time elapses. According to an embodiment, a notification message display or the like may be output while a photographing function is provided regardless of whether the first timer time elapses or the second timer time elapses, and will be described in more detail with reference to drawings that will be described below.

According to various embodiments, when an object 1615 that is associated with the photographing function is selected, the electronic device 130 may be provided with a display of a screen (e.g., a guide screen 1620 that will be described later) for executing the photographing function and/or photographing.

According to various embodiments, the display state of the object 1615 and/or a possibility of selection may be determined according to whether photographing is allowed.

According to various embodiments, whether or not photographing is currently allowed may be visually provided according to a display state of the object 1615. The display state may mean a visual attribute (e.g., a shape, a color, presence of display) of the object 1615. For example, the electronic device 130 may set a visual attribute of the object 1615 as a first attribute when the photographing is in the non-allowable state, and may set an attribute of the object 1615 as a second attribute that is different from the first attribute when the photographing is in the allowable state. According to another embodiment, whether or not photographing is currently allowed may be provided through various means (e.g., display of a notification message, output of a vibration effect, output of a voice message, and/or display of a guide screen 1620 that will be described later). Then, the notification message (e.g., “Recommended time out”, or “Recommended time out. Do you still want to photograph?”) that is provided when the second reaction time elapses may be provided to a surrounding area of the object 1615. The timer screen 1620 and various pieces of information and/or the object 1615 described above will be described in more detail with reference to drawings described below. Meanwhile, without being limited to the described and/or illustrated examples, and the present disclosure may be configured to provide contents (e.g., an object, a notification message, and the like) that indicate whether photographing is allowed on the guide screen 1620 and/or the result screen 1630. For example, a visual attribute of the object 1623 for photographing on the guide screen 1620 may be changed, or a notification message may be provided. As another example, the notification message may be provided on the result screen 1630.

According to various embodiments, the electronic device 130 may provide a photographing guide function in operation 1550. According to various embodiments, the electronic device 130 may provide at least one guide object for guiding a specific photographing condition based on the acquired product information. For example, a specific photographing environment (or a specific photographing condition) may include a photographing position (e.g., an inclination (hereinafter, an absolute inclination or an absolute angle)) of the electronic device 130 with respect to the ground, or an angle (hereinafter, a relative inclination or a relative angle) between the electronic device 130 and the in vitro diagnostic device 110 and/or a disposition direction of the in vitro diagnostic device 110. According to various embodiments, the electronic device 130 may identify whether a specific photographing condition is satisfied by providing at least one guide object. By photographing the in vitro diagnostic device 110 while the electronic device 130 satisfies a specific photographing environment, the accuracy of the diagnosis result based on the image of the in vitro diagnostic device 110 may be improved.

Referring to FIG. 16(b), the guide screen 1620 may include a first guide object 1621a for guiding the photographing position of the in vitro diagnostic device 110, a third guide object 1621b for guiding the disposition direction of the in vitro diagnostic device 110, and/or a photographing object 1623 that is configured to provide a photographing function. Although not illustrated, the guide screen 1620 may further include a second guide object for guiding the photographing angle of the in vitro diagnostic device 110, and this will be described in more detail with reference to drawings described later. Meanwhile, although not illustrated, a preview image that is acquired through the camera 1401 of the electronic device 130 may be displayed through the guide screen 1620, and at least one of the above-described guide objects 1621a and 1621b may be displayed together with the preview image (e.g., displayed on the preview image or displayed to overlap a portion of the preview image), and this will be described in more detail with reference to drawings described later. According to various embodiments, when the photographing object 1623 that is configured to provide a photographing function is selected, the electronic device 130 may be provided with display of a screen (e.g. the result screen 1630 that will be described later) that provides execution of a photographing function and/or a diagnosis result.

According to various embodiments, the electronic device 130 may acquire an image (hereinafter, referred to as a first image) of the in vitro diagnostic device in operation 1570. According to various embodiments, the electronic device 130 may acquire an image including at least one in vitro diagnostic device 110 (or at least one pad of at least one in vitro diagnostic device 110) by using the camera 1401. According to an embodiment, the electronic device 130 may provide (or transmit) the image to the server 140 for analysis of the acquired image, but without being limited to the described example, and the analysis may be performed by the electronic device 130.

According to various embodiments, the electronic device 130 may provide a result screen in operation 1590. According to various embodiments, the electronic device 130 may acquire result information based on at least one reaction area (e.g., a control area 223 of FIG. 2A, and/or a test area 221 of FIG. 2A), based on the acquired first image or the image acquired from the first image (hereinafter, referred to as a second image), and provide (e.g., display) a result screen that indicates the acquired result information. For example, the result information may include analysis information (e.g., a color value and/or a concentration of a specific line (e.g., a control line and/or a test line) that is expressed in the reaction area when the material in the specimen “D” reacts with the sample material) and/or diagnosis result information (e.g., Information that indicates a value of biometric information measurement value (e.g., hormones) that is to be diagnosed, and/or information that indicates whether it is negative/positive for the disease).

Referring to FIG. 160, the result screen 1630 may include a first image 1631 for the in vitro diagnostic device 110 (or a part of the in vitro diagnostic device 110), a second image 1633 (e.g., the image for the sample pad 220c) that is acquired based on the first image 1631, and/or an object 1635 that indicates a diagnosis result. Meanwhile, although not illustrated, at least one graphic object that is associated with the result information (e.g., analysis information and/or diagnosis result information) for emphasizing the at least one inspection line (e.g., a control line and/or a test line) may be displayed on the first image 1631, the second image 1633, and/or an area of the result screen 1630, and this will be described in more detail with reference to the drawings that will be described later. According to an embodiment, the electronic device 130 may provide (e.g., transmit) an image (e.g., the first image 1631, and/or the second image 1633) including at least a part of the in vitro diagnostic device 110 to the server 140, and may acquire result information based on at least one inspection line based on the image (e.g., the first image 1631 and/or the second image 1633) provided by the server 140 to provide (e.g., transmit) it to the electronic device 130, but without being limited to the described example, and the result information based on the analysis may be acquired by the electronic device 130. The electronic device 130 may display a result screen 1630 including the provided result information and at least one graphic object (not illustrated) that is associated with the provided result information (e.g., analysis information and/or diagnosis result information).

Hereinafter, examples of operations of the electronic device 130 according to various embodiments will be further described.

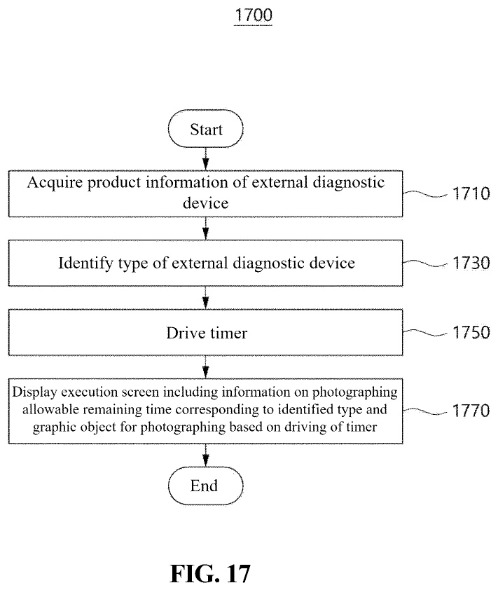

FIG. 17 is a flowchart 1700 illustrating a method of providing a timer function based on product information by the electronic device 130 (e.g., the processor 1409) according to various embodiments. According to various embodiments, the operations illustrated in FIG. 17 may be performed in various orders without being limited to the order illustrated. Furthermore, according to various embodiments, more operation than the operations illustrated in FIG. 17 or at least one operation that is less than the operations may be performed.

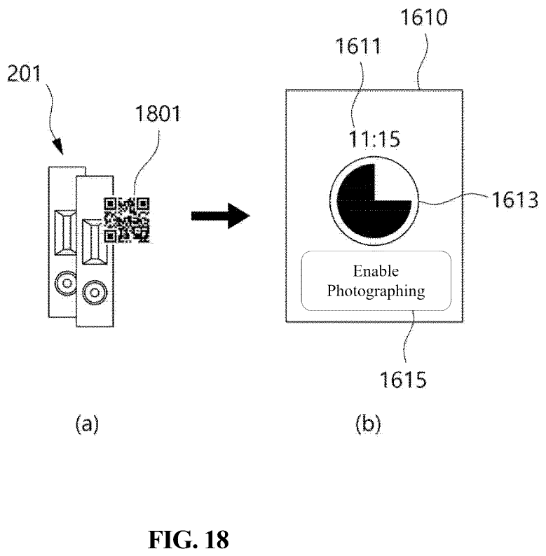

FIG. 18 is a view illustrating a method of providing a timer function based on product information by the electronic device 130 (e.g., the processor 1409) according to various embodiments.

According to various embodiments, the electronic device 130 may acquire product information of the in vitro diagnostic device 110 in operation 1710. For example, the product information may include identification information of the in vitro diagnostic device 110, such as the product/serial number of the in vitro diagnostic device 110, information on the reaction time (e.g., the first reaction time and/or the second reaction time) for providing the diagnostic result information, information on the photographing allowable time (e.g., the first photographing allowable time and/or the second photographing allowable time), information of the type of in vitro diagnostic device 110, information on various components (e.g., pads) that constitute the in vitro diagnostic device 110, the reaction areas (e.g., the test area and the control area), and a surrounding object (e.g., a specific pattern and/or engraving), and/or product information that is used to provide diagnostic result information, such as information on at least one guide object. According to various embodiments, the electronic device 130 may acquire an image of the in vitro diagnostic device 110 and/or the package (e.g., the holder 120 of FIG. 7A) of the in vitro diagnostic device 110 by using the camera 1401, and may acquire product information of the in vitro diagnostic device 110 based on the acquired image (e.g., acquire product information based on at least one visual mark (e.g., a QR code) included on an outer surface of the holder 120 detected from the image). Referring to FIG. 18(a), at least one visual mark (e.g., a QR code) 1801 may be included in the outer surface of the in vitro diagnostic device 110. The electronic device 130 may acquire an image including the QR code 1801, and acquire at least a part of the above-described product information based on the image including the QR code 1801. According to an embodiment, the electronic device 130 may acquire at least a part of the above-described product information by receiving it directly from the user of the electronic device 130.

According to various embodiments, the electronic device 130 may acquire the type of the in vitro diagnostic device 110 in operation 1730. For example, the electronic device 130 may identify the type of the in vitro diagnostic device 110 based on the identification information of the in vitro diagnostic device 110. Alternatively, the electronic device 130 may identify the type of the in vitro diagnostic device 110 by acquiring information on the in vitro diagnostic device 110 and/or the type of the in vitro diagnostic device 110 based on the image of the package (e.g., the holder 120 of FIG. 7A) of the in vitro diagnostic device 110.

According to various embodiments, the electronic device 130 may operate the timer in operation 1750. According to various embodiments, the electronic device 130 may drive the timer based on the timer time. For example, the timer time may include a first timer time and/or a second timer time, and the first and second timer times may be set to correspond to the first and second photographing allowable times, respectively (e.g., to be the same). The first photographing allowable time and/or the second photographing allowable time may be preset based on the first reaction time and/or the second reaction time that is required to provide diagnosis result information. For example, the first timer time (or the first photographing allowable time) may be set to be the same as the first reaction time (e.g., a minimum time, by which a result of a reaction between the specimen “D”, to which a specific inspection material is added, and the sample material of the sample pad 220c, may be relied upon after the specimen “D” is introduced (or the accuracy, such as the diagnosis sensitivity becomes a threshold value or more)), or to be larger than the first reaction time by a specific time (e.g., a specific ratio of the first reaction time). In an embodiment, when the in vitro diagnostic device 110 is a diagnostic device for detecting a corona antigen, the first timer time may be set to 15 minutes. According to an embodiment, the first reaction time and/or the second reaction time may be changed based on the surrounding environment temperature, or may be set such that he first photographing allowable time and/or the second photographing allowable time is adjusted based on the surrounding environment temperature. For example, the photographing allowable times (e.g., the first photographing allowable time and the second photographing allowable time) may be adjusted to be inversely proportional to the surrounding environment temperature (e.g., the photographing allowable time becomes shorter when the surrounding environment temperature is higher), but may be configured in contrary to the examples described above. The electronic device 130 may identify a temperature value that is identified based on a sensor (not illustrated) for detecting a temperature, and/or may identify a temperature value based on information (e.g., a color temperature) that is related to a color that is identified from an image captured by using the camera 1401. For example, the second timer time (or the second photographing allowable time) may be set to be the same as the second reaction time (e.g., the maximum time, by which a result of a reaction of the specimen “D”, to which a specific inspection material is added after the inspection substance is introduced), and the sample material of the sample pad 220c or to be shorter than the second reaction time by a specific ratio. In an embodiment, when the in vitro diagnostic device 110 is a diagnostic device for detecting a corona antigen, the second timer time may be set to 30 minutes. According to various embodiments, the timer time may be different depending on the type of the in vitro diagnostic device 110. For example, when the in vitro diagnostic device 110 is of the first type, the timer time may include a timer time having at least one first timer value corresponding to the first type, and when the in vitro diagnostic device 110 is of the second type, the timer time may include a timer time having at least one second timer value corresponding to the second type. According to various embodiments, as the timer value is determined to correspond to the type of the in vitro diagnostic device 110, information on the remaining time, for which photograph is allowed, included in the execution screen that will be described later, and/or a graphic object for photographing may be displayed to correspond to the type of the in vitro diagnostic device 110.

According to various embodiments, the electronic device 130 may display an execution screen (e.g., a timer screen 1610 of FIG. 16) including photographing allowable remaining time information corresponding to the identified type and a graphic object (hereinafter, a photographing object) for photographing, based on driving of the timer in operation 1770. For example, when the in vitro diagnostic device 110 is of the first type, the electronic device 130 may display information on the remaining time until at least one timer time (e.g. the first timer time and/or the second timer time) having at least one first timer value corresponding to the first type, and an execution screen including a first graphic object (e.g., a photographing object) for photographing. When the in vitro diagnostic device 110 is of the second type, the electronic device 130 may display information on a remaining time until at least one timer time (e.g., the first timer time and/or the second timer time) having at least one second timer value corresponding to the second type, and an execution screen including a second graphic object (e.g., a photographing object) for photographing.

FIG. 18(b) illustrates a timer screen 1610 at a time point after a specific time has elapsed after the timer was driven.

According to various embodiments, the object 1611 of the timer screen 1610 may indicate the first remaining time. For example, the first remaining time indicates a time that remains from the current time point to the first timer time (or the first photographing allowable time), and a photographing function using the camera 1401 may be allowed from a time point, at which the first remaining time becomes 0 (or 0:00). For example, when the first timer time set for the in vitro diagnostic device 110 is set to 15 minutes, the illustrated object 1611 may indicate that the first remaining time is 11 minutes and 15 seconds, and 3 minutes and 45 seconds have elapsed after the timer was driven.

According to various embodiments, the object 1613 of the timer screen 1610 may indicates the first remaining time in a visual form. For example, the object 1613 may be configured in a circular shape, a fan shape, or other various shapes. When object 1613 is configured in a fan shape, a ratio (e.g., 75%) of the first remaining time to the first timer time may be provided based on the angle (e.g., 270 degrees) of the fan shape.

According to various embodiments, the timer screen 1610 may include at least one graphic object for visually indicating the second timer time (or the second photographing allowable time) and/or the second remaining time. According to an embodiment, when the second remaining time reaches a specific value (e.g., 0) around the object 1615 of the timer screen 1610, a notification message may be provided. According to another embodiment, among a plurality of objects of the timer screen 1610, at least one first object may indicate the first timer time and/or the first remaining time, and the remaining at least one object may be configured to indicate the second timer time and/or the second remaining time. As an example, the object 1613 may be set to indicate the second remaining time in a visual form. For example, the second remaining time indicates a time that remains from the current time point to the second timer time (or the second photographing allowable time), and a photographing function using the camera 1401 may be allowed from until the second remaining time becomes 0 (or 0:00). In this case, the object 1613 may indicate that about 75% of the second timer time (or the second photographing allowed time) remains as the second remaining time. Accordingly, information on the first remaining time may be provided through the object 1611 and information on the second remaining time may be provided through the object 1613, so that a time point, at which photographing is possible, and a time point, at which photographing is no longer possible, may be provided to the user together. According to an embodiment, after the second timer time has elapsed (e.g., after the second remaining time becomes 0 (or 0:00)), the electronic device 130 may perform at least one operation according to the type of the execution screen that is being displayed on the display module 1403, and this will be described in more detail with reference to the drawings described below.

According to various embodiments, the object 1615 of the timer screen 1610 may be configured to provide a function that is associated with photographing. For example, when the object 1615 is selected, the electronic device 130 may execute a photographing function or display a screen (e.g., the guide screen 1620) for photographing. According to an embodiment, the object 1615 (or a photographing operation) may be enabled after the first timer time (e.g., when all of the first remaining time has elapsed) or after the first timer time and before the second timer time (e.g., when the entire second remaining time has not elapsed), and the other time may be disenabled, and this will be described in more detail with reference to the drawings described below.

Hereinafter, examples of operations of the electronic device 130 according to various embodiments will be further described.

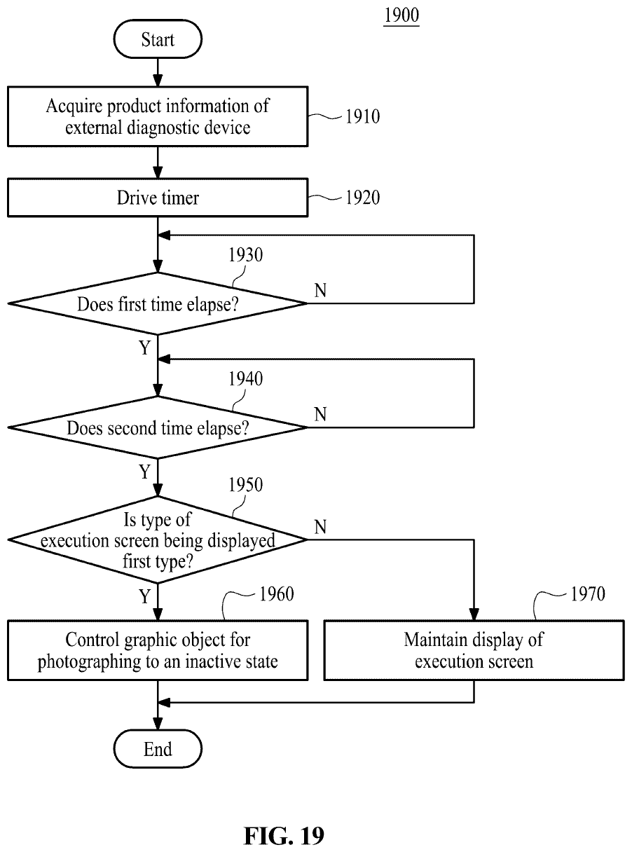

FIG. 19 is a flowchart 1900 illustrating a method of providing a plurality of timer functions based on product information by the electronic device 130 (e.g., the processor 1409) according to various embodiments. According to various embodiments, the operations illustrated in FIG. 19 may be performed in various orders without being limited to the order illustrated. Furthermore, according to various embodiments, more operation than the operations illustrated in FIG. 19 or at least one operation that is less than the operations may be performed.

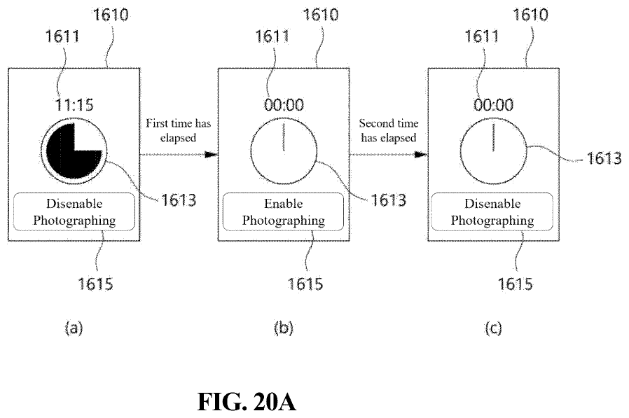

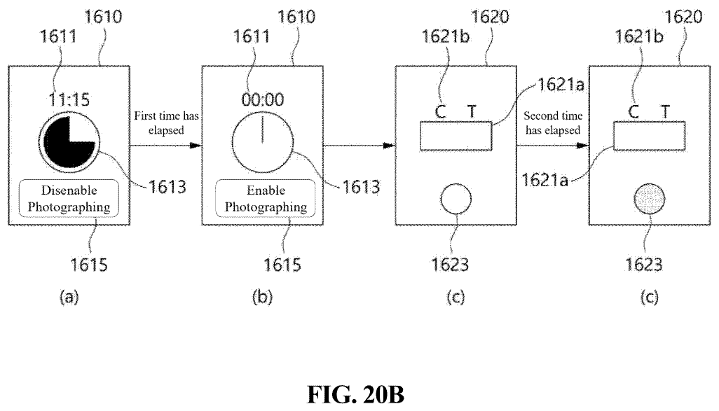

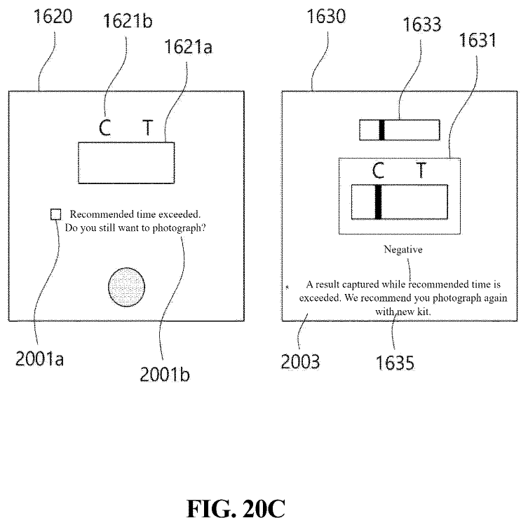

FIGS. 20A to 20C illustrate screens, in which the electronic device 130 (e.g., the processor 1409) provides a plurality of timer functions based on product information according to various embodiments.

According to various embodiments, the electronic device 130 may acquire product information of the in vitro diagnostic device 110 in operation 1910. For example, the product information may include identification information of the in vitro diagnostic device 110, such as the product/serial number of the in vitro diagnostic device 110, information on the reaction time (e.g., the first reaction time and/or the second reaction time) for providing the diagnostic result information, information on the photographing allowable time (e.g., the first photographing allowable time and/or the second photographing allowable time), information of the type of in vitro diagnostic device 110, information on various components (e.g., pads 220) that constitute the in vitro diagnostic device 110, the reaction areas (e.g., the test area 211 of FIG. 2A and the control area 213 of FIG. 2A), and a surrounding object (e.g., a specific pattern and/or engraving), and/or product information that is used to provide diagnostic result information, such as information on at least one guide object.

According to various embodiments, the electronic device 130 may drive the timer in operation 1920. According to various embodiments, the timer may be operated based on the first timer time and the second timer time, and a plurality of timer functions may be provided.

According to various embodiments, the electronic device 130 may identify whether the first time has elapses after the timer was driven in operation 1930. For example, the first time may be the first timer time. According to various embodiments, the electronic device 130 may repeat operation 1930 until the first time elapses. According to an embodiment, the electronic device 130 may not allow a photographing function or may not display a screen (e.g., the guide screen 1620) for photographing until the first time elapses.

Referring to FIGS. 20A and 20B, the timer screen 1610 may include an object 1611 that indicates a first remaining time, an object 1613 that indicates a first remaining time, and/or an object 1615. FIG. 20A(a) and FIG. 20B(a) indicate a timer screen 1610 before a first time has elapsed after the timer was driven. According to various embodiments, the electronic device 130 may control the state of the object 1615 to a first state (e.g., an inactive state) before a first time elapses, or may limit the selection of the object 1615. As selection of the object 1615 is limited, even when an input for selecting the object 1615 is received, the photographing function may not be executed or/or the screen 1620 (e.g., the guide screen 1620) for photographing may not be displayed. According to an embodiment, the electronic device 130 may display a warning message (e.g., “not enough time has elapsed”) together with the object 1615 while controlling the state of the object 1615 to an active state before a first time has elapsed after the timer was driven.

According to various embodiments, the electronic device 130 may identify whether the first time has elapses after the timer was driven in operation 1940 when it is identified that the second time has elapsed. For example, the second time may be the second timer time. According to various embodiments, when it is identified that the first time has elapses, the electronic device 130 may allow a photographing function or may display a screen (e.g., the guide screen 1620) for photographing. According to an embodiment, the electronic device 130 may display a message that indicates that photographing is possible while the photographing object 1615 or 1623 is controlled to a second state (e.g., an active state) after the first time has elapsed after the timer was driven, or may automatically execute a photographing function (e.g., a reservation photographing function).

FIG. 20A(b) and FIG. 20B(a) indicate a timer screen 1610 before a second time has elapsed after the timer was driven. According to various embodiments, while the timer screen 1610 is displayed, the electronic device 130 may control the object 1615 to a second state (e.g., an active state) until a second time has elapsed after a first time elapsed, and/or may allow selection of the object 1615. As selection of the object 1615 is allowed, when an input for selecting the object 1615 is received, the photographing function may be executed or/or the screen (e.g., the guide screen 1620) for photographing may be displayed.

FIG. 20A(c) indicate a guide screen 1620 before a second time has elapsed after the timer was driven. According to various embodiments, the electronic device 130 may display the guide screen 1620 based on selection of the object 1615 after a first time has elapsed on the timer screen 1610. While the guide screen 1620 is displayed, the electronic device 130 may control the photographing object 1623 to a second state (e.g., an active state) until a second time elapses, and/or may allow selection of the photographing object 1623. Because selection of the photographing object 1623 is allowed, the electronic device 130 may execute a photographing function when an input for selecting the photographing object 1623 is received. According to an embodiment, when an input for selecting the photographing object 1623 is received when a specific photographing environment is satisfied, the electronic device 130 may execute a photographing function, and this will be described in more detail with reference to the drawings described below.

According to various embodiments, the electronic device 130 may repeat operation 1950 until the second time elapses.

According to various embodiments, when it is identified that the second time elapses, the electronic device 130 may identify whether the type of the displayed execution screen is of the first type in operation 1950. For example, the first type of the execution screen may include an execution screen (e.g. the timer screen 1610) that provides a timer function and/or an execution screen (e.g., the guide screen 1620) that provides a guide function. For example, the second type of execution screen may include the execution screen other than the above-described first type of execution screen. FIG. 20A(c) indicate a timer screen 1610 after a second time has elapsed after the timer was driven. FIG. 20A(d) indicate a guide lower surface 1620 after the second time has elapsed after the timer was driven. As an example, the second type of execution screen may include an execution screen (e.g., the result screen 1630 of FIG. 20A(c)) that indicates result information based on at least one inspection line.

According to various embodiments, when it is identified that the type of the execution screen that is being displayed is of the first type, the electronic device 130 may control the graphic object for photographing to a disenabled state in operation 1960. For example, referring to FIG. 20A(c), the electronic device 130 may control the object 1615 to an inactive state when the execution screen that is being displayed is the execution screen (e.g., the timer screen 1610) that provides a timer function. Referring to FIG. 20B(d), the electronic device 130 may control the photographing object 1623 to an inactive state when the execution screen that is being displayed is an execution screen (e.g., the guide screen 1620) provides a guide function. According to an embodiment, the electronic device 130 may allow the photographing function even when the second time has elapsed. For example, referring to FIG. 20C(a), the electronic device 130 may display a graphic object that is associated with the second time when the execution screen that is being displayed is an execution screen (e.g., the guide screen 1620) that provides a guide function. For example, the graphic object associated with the second time may include a photographing allowing object 2001a and a photographing guide message (e.g., “the recommended time was exceeded. Do you still want to photograph?”) 2001b. When the photographing allowing object 2001a and/or the photographing guide message 2001b is selected, the electronic device 130 may control the object 1615 to a second state (e.g., an active state). When the object 1615 in the second state is selected, the electronic device 130 may execute a photographing function. Alternatively, when the object 1615 in the inactive state is selected, the photographing allowing object 2001a and/or the photographing guide message 2001b is displayed, and when the photographing allowing object 2001a and/or the photographing guide message 2001b is selected, the object 1615 may be controlled to an active state. As another example, when the execution screen (e.g., the guide screen 1620) that is being displayed provides a guide function, the electronic device 130 may not control the object 1615 to an inactive state even when the second time elapses. In this case, when an execution screen (e.g., the result screen 1630 of FIG. 20C(b)) indicating result information based on at least one inspection line included in the image captured after the second time period has elapsed is displayed, a graphic object associated with the second time may be displayed together with an object (e.g., a text) 1635 that indicates diagnosis result information. For example, a graphic object associated with the second time may include a message (e.g., “This is the result captured while the recommended time was exceeded. I recommend re-photographing with a new kit”) (2003) that indicates that the graphic object is information based on an image captured after the second time has elapsed.

According to various embodiments, when it is identified that the type of the execution screen that is being displayed is an execution screen (e.g., the execution screen of the second type) that is not of the first type, the electronic device 130 may maintain the display of the execution screen in operation 1970. For example, when the execution screen that is being displayed is the execution screen (e.g., the result screen 1630) that indicates result information based on at least one inspection line, the electronic device 130 may maintain the display of the execution screen (e.g., the result screen 1630) without changing it.

Hereinafter, examples of operations of the electronic device 130 according to various embodiments will be further described.

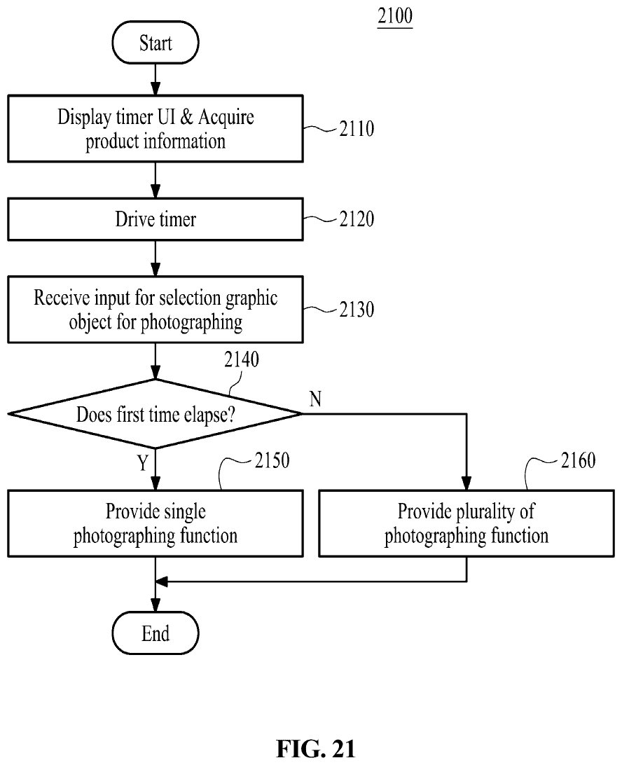

FIG. 21 is a flowchart 2100 illustrating a method of providing a continuous photographing function based on a timer in the electronic device 130 (e.g., the processor 1409) according to various embodiments. According to various embodiments, the operations illustrated in FIG. 21 are not limited to the order illustrated, and may be performed in various orders. Furthermore, according to various embodiments, at least one operation that is less or more than the operations illustrated in FIG. 21 may be performed.

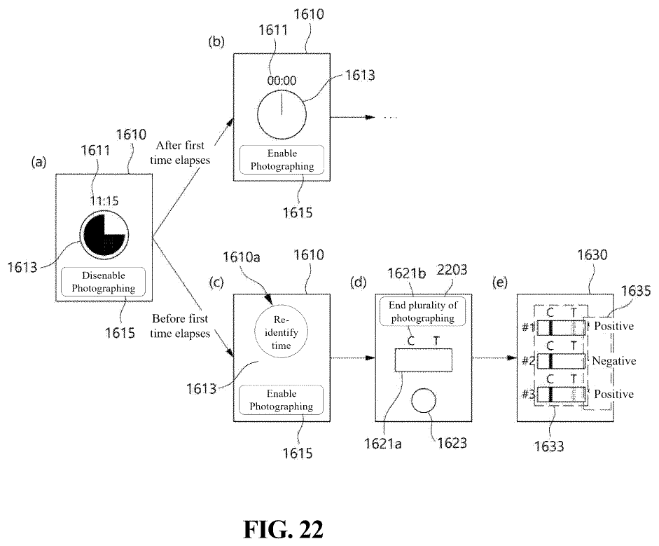

FIG. 22 illustrates screens, in which the electronic device 130 (e.g., the processor 1409) provides a continuous photographing function based on a timer according to various embodiments.

According to various embodiments, the electronic device 130 may display the timer UI in operation 2110. For example, the electronic device 130 may display a timer function on the screen (e.g., the timer screen 1610 of FIG. 22) based on the acquired product information of the in vitro diagnostic device 110. For example, the product information may include identification information of the in vitro diagnostic device 110, such as the product/serial number of the in vitro diagnostic device 110, information on the reaction time (e.g., the first reaction time and/or the second reaction time) for providing the diagnostic result information, information on the photographing allowable time (e.g., the first photographing allowable time and/or the second photographing allowable time), information of the type of in vitro diagnostic device 110, information on various components (e.g., pads 220 of FIG. 2A) that constitute the in vitro diagnostic device 110, the reaction areas (e.g., the test area 211 of FIG. 2A and the control area 213 of FIG. 2A), and a surrounding object (e.g., a specific pattern and/or engraving), and/or product information that is used to provide diagnostic result information, such as information on at least one guide object.

According to various embodiments, the electronic device 130 may drive the timer in operation 2130. According to various embodiments, the timer may be operated based on the first timer time and/or the second timer time, and at least one timer function may be provided.

According to various embodiments, the electronic device 130 may receive an input for selecting a graphic object (e.g., the object 1615) for photographing, in operation 2130. According to various embodiments, the state of the graphic object (e.g. object 1615) for photographing may be a second state (e.g., an active state) or a first state (e.g., an inactive state) depending on whether the first time (e.g., the first timer time) has elapsed. For example, before the first time elapses after the timer is driven, the state of the graphic object (e.g. the object 1615) for photographing may be a first state (e.g., an inactive state), as illustrated in FIG. 22A. After the first time has elapses after the timer was driven, the state of the graphic object (e.g. the object 1615) for photographing may be controlled to the second state (e.g., an active state), as illustrated in FIG. 22(b).

According to various embodiments, when receiving an input for selecting a graphic object (e.g., the object 1615) for photographing, the electronic device 130 may identify whether the first time has elapsed after the timer was driven in operation 2140. For example, the first time may be the first timer time.

According to various embodiments, when receiving an input for selecting the graphic object (e.g., the object 1615) for photographing, the electronic device 130 may provide a single photographing function when the first time has elapsed because the timer was driven in operation 2150. For example, the electronic device 130 may provide (e.g., display) a screen (e.g., the guide screen 1620 of FIG. 16(b) for photographing, and/or execute a photographing function. According to an embodiment, when receiving an input for selecting the graphic object (e.g. and the object 1615) for photographing, the electronic device 130 may perform operations 1940 of FIG. 19 and below when the first time has elapsed after the timer was driven.

According to various embodiments, when receiving an input for selecting the graphic object (e.g., the object 1615) for photographing, the electronic device 130 may provide a plurality of photographing functions when the first time has not elapsed because the timer was driven in operation 2160.

Referring to FIG. 22(c), when the graphic object (e.g. and the object 1615) for photographing is selected while the first time has not elapsed, the electronic device 130 may display a warning message (e.g., “identify time again”) 1610a for photographing, and may control a state of the graphic object (e.g., the object 1615) for photographing to a second state (e.g., an active state). Thereafter, referring to FIG. 22(c), when the graphic object (e.g., the object 1615) for photographing is selected, the electronic device 130 may display a guide screen 1620 that provides a plurality of photographing functions. Referring to FIG. 22(d), the guide screen 1620 that provides a plurality of photographing functions may include an object 2203 that indicates that the plurality of photographing functions are provided. According to an embodiment, when the object 2203 is selected, the plurality of photographing functions may be ended. In the guide screen 1620 that provides a plurality of photographing functions, even when the first time has elapsed after the timer was driven, the photographing object 1623 in an active state and may be selected. When the photographing object 1623 is selected, the electronic device 130 may execute a photographing function, and may display diagnosis results based on at least one inspection line of the plurality of in vitro diagnostic devices on an execution screen (e.g., the result screen 1630 of FIG. 20A(c)) that indicates result information. Referring to FIG. 22(e), diagnosis results of three in vitro diagnostic devices #1, #2, and #3 are illustrated. As illustrated, second images 1633 of the first, second, and third in vitro diagnostic devices may be displayed. As well, objects 1635 that indicates diagnosis result information corresponding to the first, second, and third in vitro diagnostic devices #1, #2, and #3 may be displayed. According to various embodiments, diagnosis result information based on at least one inspection line of the plurality of in vitro diagnostic devices may be displayed in a photographing order. According to various embodiments, the electronic device 130 may receive identification information (e.g., a patient, a test item, and the like) of each of the in vitro diagnostic devices before providing a plurality of photographing functions, and display the received identification information together with the above-described diagnosis result information.

Hereinafter, examples of operations of the electronic device 130 according to various embodiments will be further described.

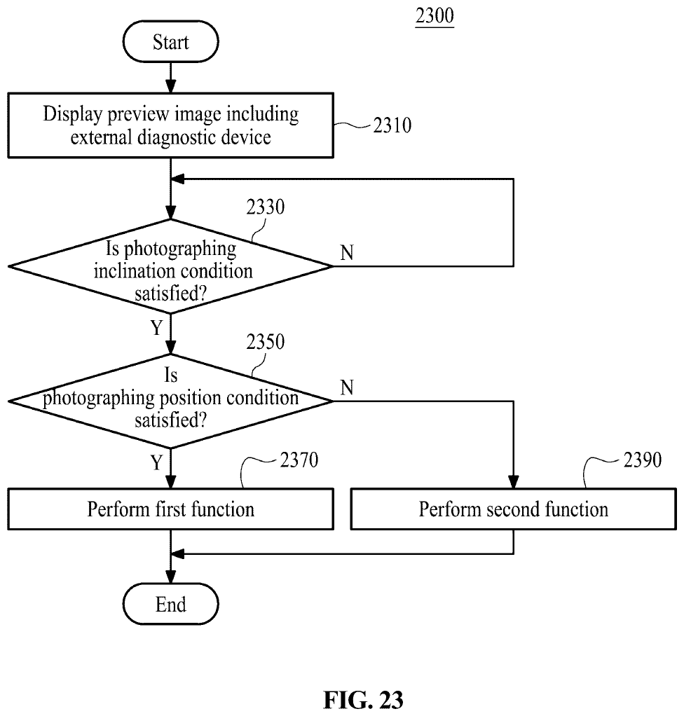

FIG. 23 is a flowchart 2300 illustrating a method of guiding a photographing position and/or a photographing angle of the in vitro diagnostic device 110 by the electronic device 130 (e.g., processor 1409), according to various embodiments. According to various embodiments, the operations illustrated in FIG. 23 are not limited to the order illustrated, and may be performed in various orders. Furthermore, according to various embodiments, at least one operation that is less or more than the operations illustrated in FIG. 23 may be performed.

According to various embodiments, the electronic device 130 may display a preview image including the in vitro diagnostic device 110 in operation 2310. For example, the electronic device 130 may display a screen (e.g., the guide screen 1620 of FIG. 16) for providing a photographing function, and display an image that is acquired using the camera 1401 in the form of a preview image through a screen (e.g., the guide screen 1620 of FIG. 16) for providing a photographing function. According to an embodiment, the electronic device 130 may display a screen (e.g., the guide screen 1620 of FIG. 16) for providing a photographing function when the object 1615 is selected after the first time (e.g., a first timer time) has elapsed in the state, in which a timer screen (e.g., the timer screen 1610 of FIG. 16) is displayed. According to various embodiments, at least one guide object may be included on the screen (e.g., the guide screen 1620 of FIG. 16) for providing the photographing function, and the electronic device 130 may display at least one guide object in a shape and/or a size corresponding to the product information, based on the acquired product information (e.g., identification information of the in vitro diagnostic device 110, such as the product/serial number of the in vitro diagnostic device 110, information on the reaction time (e.g., the first reaction time and/or the second reaction time) for providing the diagnostic result information, information on the photographing allowable time (e.g., the first photographing allowable time and/or the second photographing allowable time), information of the type of in vitro diagnostic device 110, information on various components (e.g., pads) that constitute the in vitro diagnostic device 110, the reaction areas (e.g., the test area and the control area), and a surrounding object (e.g., a specific pattern and/or engraving), and/or information on at least one guide object). According to various embodiments, the at least one guide object may be displayed together with the preview image (e.g., to overlap at least a portion of the preview image), and this will be described in more detail with reference to drawings described later.

According to various embodiments, the electronic device 130 may determine whether the photographing inclination condition is satisfied in operation 2330. For example, the photographing inclination condition may include whether the photographing angle (e.g., the absolute inclination or the relative inclination) satisfies a preset angle (e.g., 10 degrees), or a preset angle range (e.g., 5 to 30 degrees). In more detail, the photographing inclination condition may include whether the absolute inclination of the electronic device 130 satisfies a preset angle or a preset angle range, or whether the relative inclination of the electronic device 130 satisfies a preset angle or a preset angle range. According to various embodiments, whether the photographing inclination condition is satisfied may be identified based on the first guide object 1621a, the second guide object 2501, and/or a sensing value of at least one sensor 1405 (e.g., an inertia sensor) of the electronic device 130 described below, and this will be described in more detail with reference to the drawings described below. According to various embodiments, when the photographing inclination condition is not satisfied, the electronic device 130 may repeat operation 2330. According to an embodiment, the electronic device 130 may identify whether the photographing inclination condition is satisfied through the flash function. For example, the electronic device 130 may execute a flash function, and the light that is output from at least one light source (not illustrated) of the camera 1401 may measures the intensity of light (hereinafter, referred to as reflected light) that is reflected to return by the in vitro diagnostic device 110, and when the measured intensity of reflected light is less than or equal to a specified intensity, it may be identified that the photographing inclination condition is satisfied. In this case, it may be identified whether the photographing inclination condition is satisfied even without using the second guide object 2501.

According to various embodiments, when it is identified that the photographing inclination condition is satisfied, the electronic device 130 may identify whether the photographing position condition is satisfied in operation 2350. For example, the photographing position condition may include whether a disposition direction of the electronic device 130 with respect to the in vitro diagnostic device 110 is a preset direction, and/or whether a distance between the in vitro diagnostic device 110 and the electronic device 130 satisfies a preset distance. According to various embodiments, whether the photographing position condition is satisfied may be identified based on a first guide object 1621a that will be described later, and this will be described in more detail with reference to the drawings described later. According to an embodiment, the electronic device 130 may identify whether the photographing position condition is satisfied before identifying whether the photographing inclination condition is satisfied, and may identify whether the photographing inclination condition is satisfied when the photographing position condition is identified. In this case, when the photographing position condition is not satisfied, the electronic device 130 may repeat operation 2350 and perform operation 2330 when the photographing position condition is satisfied. When the photographing inclination condition is satisfied, the electronic device 130 may perform operation 2370, and when the photographing inclination condition is not satisfied, may perform operation 2390. According to an embodiment, when the photographing inclination condition is satisfied, the electronic device 130 may execute a flash function of the camera. When a flash function of the camera is executed while a specific photographing inclination condition is satisfied, a degree, to which light (hereinafter, reflected light) output from at least one light source of the camera 1401 is reflected to return by the in vitro diagnostic device 110 is received by the camera 1401, may be less than or equal to a preset reference. Accordingly, an influence of reflected light during photographing of the in vitro diagnostic device 110 may be decreased.

According to various embodiments, when the photographing position condition is satisfied, the electronic device 130 may perform the first function in operation 2370. For example, the first function may include enabling of the photographing object 1623 and/or execution of the photographing function. According to an embodiment, the first function may include executing a flash function of a camera. When the flash function of the camera is executed while a specific photographing inclination condition is satisfied, the degree, to which reflected light is received by the camera 1401, may be less than or equal to a preset reference. Accordingly, an influence of reflected light during photographing of the in vitro diagnostic device 110 may be decreased.

According to various embodiments, when the photographing position condition is not satisfied, the electronic device 130 may perform the second function in operation 2390. For example, the second function may include disenabling of the photographing object 1623 and/or display of a notification message (e.g., a message that indicates that photographing is impossible).

Hereinafter, examples of operations of the electronic device 130 according to various embodiments will be further described.

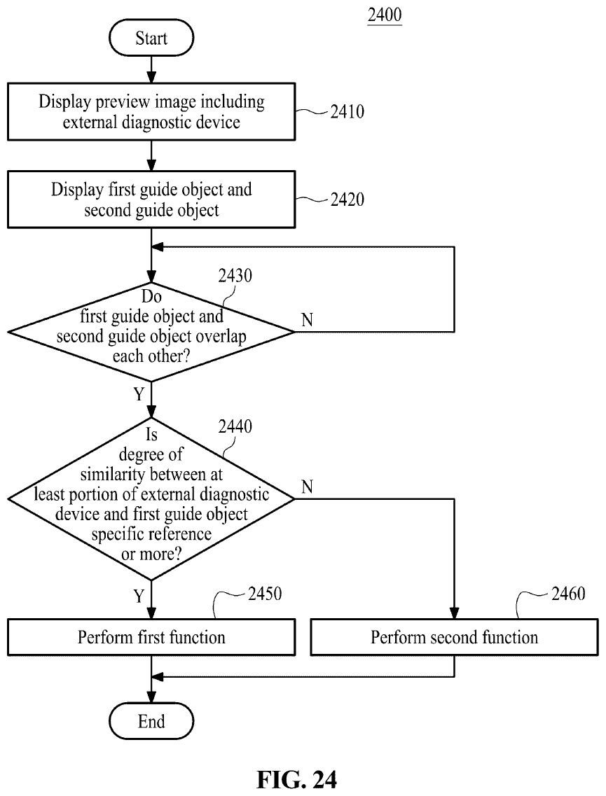

FIG. 24 is a flowchart 2400 illustrating an example of a method of guiding a photographing position and/or a photographing angle of the in vitro diagnostic device 110 by the electronic device 130 (e.g., processor 1409), according to various embodiments. According to various embodiments, the operations illustrated in FIG. 24 are not limited to the order illustrated, and may be performed in various orders. Furthermore, according to various embodiments, at least one operation that is less or more than the operations illustrated in FIG. 24 may be performed.

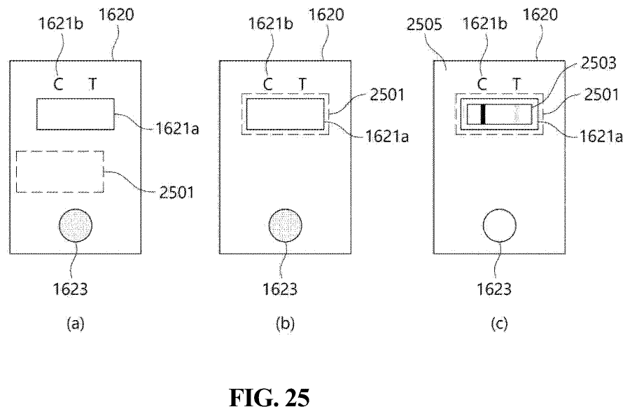

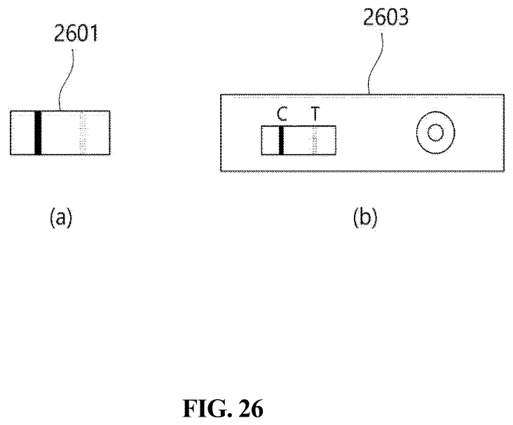

FIG. 25 illustrates screens for guiding a photographing position and/or a photographing angle of the in vitro diagnostic device 110 by the electronic device 130 (e.g., processor 1409), according to various embodiments. FIG. 26 illustrates various implementations of the second guide object 2501 according to various embodiments.

According to various embodiments, the electronic device 130 may display a preview image including the in vitro diagnostic device 110 in operation 2410.

According to various embodiments, the electronic device 130 may display a first guide object and a second guide object in operation 2420.

Referring to FIG. 25, a first guide object 1621a for guiding a photographing position, a second guide object 2501 for guiding a photographing angle, and a third guide object 1621b for guiding a disposition direction of the in vitro diagnostic device 110 are illustrated. The illustrated third guide object 1621b may be omitted. According to various embodiments, the second guide object 2501 may be displayed to correspond to an inclination (e.g., an absolute inclination) of the electronic device 130. For example, the electronic device 130 may identify the inclination (e.g., an absolute inclination) of the electronic device 130 based on sensing values that are acquired from at least one inertia sensor, and may display the second guide object 2501 such that it corresponds to the identified inclination. In more detail, when the electronic device 130 is rotated in a first direction (e.g., a roll direction), the electronic device 130 may move and display the second guide object 2501 in a leftward or rightward direction on the guide screen 1520. When the electronic device 130 is rotates in a second direction (e.g., a pitch direction), the electronic device 130 may move and display the second guide object 2501 in an upward or downward direction. According to various embodiments, the second guide object 2501 may be configured in various forms. For example, as illustrated in FIG. 25, the second guide object 2501 may be configured to have various contours, such as lines, dotted lines, and broken lines, corresponding to the contour of the pad 2503 (e.g., the sample pad 220c of FIG. 2A) of the in vitro diagnostic device 110 (or corresponding to the first guide object 1621a). As another example, as illustrated in FIG. 26, the second guide object 2501 may be configured as an image (e.g., a translucent image 2601 illustrated in (a) of FIG. 26) corresponding to the shape of the pad 2503 of the in vitro diagnostic device 110, or may be configured as an image (e.g., a translucent image 2603 illustrated in (b) of FIG. 26) corresponding to the shape of the in vitro diagnostic device 110. According to an embodiment, the second guide object 2501 may be configured in the form of a plurality of bars that indicate an inclination (e.g., an absolute inclination) of the electronic device 130. In this case, among the plurality of bars, the first bar may be configured to indicate an inclination in the first direction (e.g., a roll direction) of the electronic device 130, and the second bar may be configured to indicate an inclination in the second direction (e.g., a pitch direction) of the electronic device 130.

According to various embodiments, the electronic device 130 may identify whether the first guide object and the second guide object overlap each other in operation 2430.

Referring to FIGS. 25(a) and 25(b), (a) illustrates a case, in which the first guide object 1621a and the second guide object 2501 do not overlap each other, and (b) illustrates a case, in which the first guide object 1621a and the second guide object 2501 overlap each other. In the electronic device 130, when the electronic device 130 is rotated in the first direction (e.g., the roll direction) and/or the second direction (e.g., the pitch direction) when the inclination state of the electronic device 130 is as in FIG. 25(a), the electronic device 130 may gradually move and display the second guide object 2501 in the left/right direction and/or the up/down direction such that it corresponds to the current inclination (e.g., the absolute inclination) of the electronic device 130. According to the movement of the electronic device 130, when the electronic device 130 is disposed in a horizontal state with respect to the ground in the first direction (e.g., the roll direction), and is disposed at a preset angle (e.g., 10 degrees) or in a preset angle range (e.g., 5 to 30 degrees) with respect to the ground in the second direction (e.g., pitch direction), the second guide object 2501 may be displayed to entirely (or partially) overlap the first guide object 1621a as illustrated in FIG. 25(b). In this case, the electronic device 130 may perform at least one operation that indicates that the photographing inclination condition is satisfied. For example, the at least one operation may include various types of visual, auditory, and tactile notifications, such as a visual effect on the first guide object 1621a and/or the second guide object 2501, a sound notification, activation of the photographing object 1623, a vibration notification, and the like. According to various embodiments, the electronic device 130 may repeat operation 2430 when it is not identified that the first guide object and the second guide object overlap each other (e.g., overlap each other by a specific degree or more).

According to various embodiments, when it is identified that the first guide object and the second guide object overlap each other (e.g., overlap each other to a specific degree or more), the electronic device 130 may identify whether the degree of similarity of the at least part of the in vitro diagnostic device 110 to the first guide object is equal to or greater than a specific reference (e.g., whether it is included within a specific range) in operation 2440. For example, referring to FIG. 25(c), a preview image 2505 including the in vitro diagnostic device 110, and a guide screen 1620, on which a first guide object 1621a, a second guide object 2501, and a third guide object 1621b are displayed, are illustrated. Even in this case, the third guide object 1621b may be omitted. At least a portion (e.g., the pad 2503) of the in vitro diagnostic device 110 may be included on the preview image 2505. The first guide object 1621a and the second guide object 2501 may be in an overlapping state (e.g., a state, in which they overlap each other to a specific degree or more) according to the movement of the electronic device 130 in the first direction (e.g., the roll direction) and/or the second direction (e.g., the pitch direction). As the electronic device 130 moves in the leftward/rightward direction, the upward/downward direction, and/or the height direction (e.g., the vertical distance direction from the electronic device 110 to the in vitro diagnostic device 110) is moved, the position of at least a portion (e.g., the pad 2503) of the in vitro diagnostic device 110 included in the preview image 2505 displayed through the guide screen 1620 may be moved. According to the movement of the electronic device 130 in the leftward/rightward direction, the upward/downward direction, and/or the height direction, the position, in which at least a portion (e.g., the pad 2503) is displayed in the preview image 2505 may be moved into the first guide object 1623a. When at least a portion (e.g., the pad 2503) of the in vitro diagnostic device 110 at least partially overlaps (or is displayed inside) the first guide object 1623a, it may be identified whether the degree of similarity of at least a portion (e.g., the pad 2503) of the in vitro diagnostic device 110 to the first guide object 1623a is equal to or greater than a specific reference. As an example, as illustrated, when the first guide object 1623a has a rectangular shape, the pad 2503 is displayed in a substantially rectangular shape, and when the size of the pad 2503 is displayed to be similar to the size of the first guide object 1623a, the electronic device 130 may identify that the degree of similarity of at least a portion (e.g., the pad 2503) of the in vitro diagnostic device 110 to the first guide object 1623a is equal to or greater than a specific reference. When the first guide object 1623a is configured to correspond to the contour of the in vitro diagnostic device 110, it may be identified whether the contour of the in vitro diagnostic device 110 is equal to or greater than a specific reference. In this case, the second guide object 2501 may also be configured to correspond to the contour of the in vitro diagnostic device 110.

According to various embodiments, the electronic device 130 may perform the first function in operation 2450 when it is identified that the degree of similarity of at least a portion of the in vitro diagnostic device 110 to the first guide object is equal to or greater than a specific reference. For example, the first function may include enabling of the photographing object 1623, execution of a photographing function, and/or execution of a flash function of the camera.

According to various embodiments, the electronic device 130 may perform the second function in operation 2460 when it is identified that the degree of similarity of at least a portion of the in vitro diagnostic device 110 to the first guide object is less than a specific reference. For example, the second function may include disenabling of the photographing object 1623 and/or display of a notification message (e.g., a message that indicates that photographing is impossible).

Hereinafter, examples of operations of the electronic device 130 according to various embodiments will be further described.

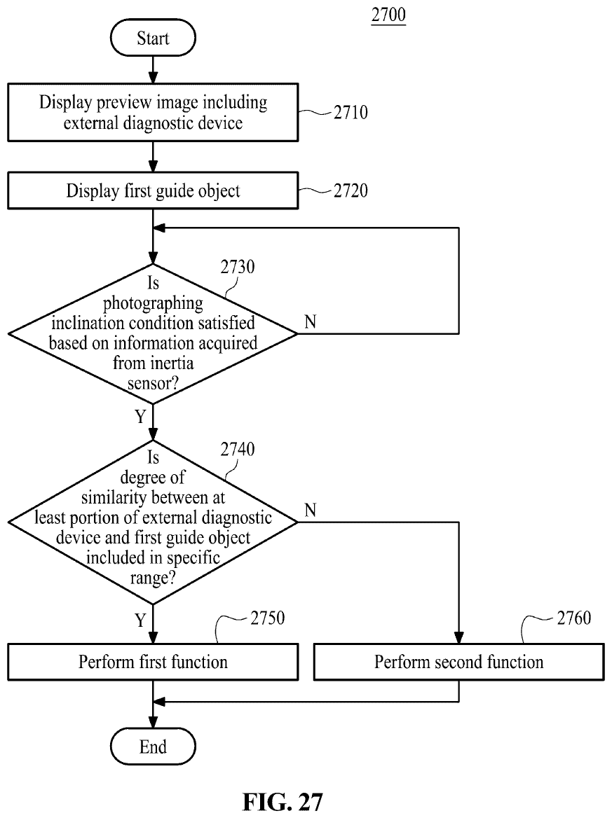

FIG. 27 is a flowchart 2700 illustrating an example of a method of guiding a photographing position and/or a photographing angle of the in vitro diagnostic device by the electronic device 130 (e.g., processor 1409), according to various embodiments. According to various embodiments, the operations illustrated in FIG. 27 are not limited to the order illustrated, and may be performed in various orders. Furthermore, according to various embodiments, at least one operation that is less or more than the operations illustrated in FIG. 27 may be performed.

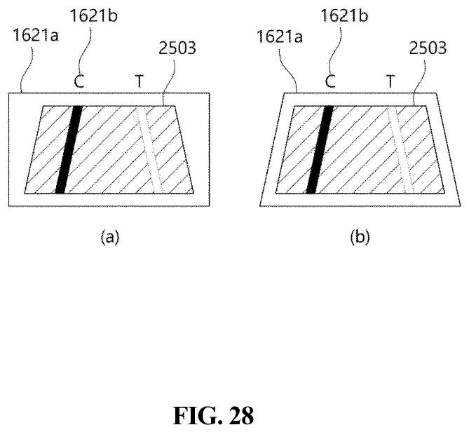

FIG. 28 illustrates screens for guiding a photographing position and/or a photographing angle of the in vitro diagnostic device 110 by the electronic device 130 (e.g., processor 1409), according to various embodiments.

According to various embodiments, the electronic device 130 may display a preview image including the in vitro diagnostic device in operation 2710.

According to various embodiments, the electronic device 130 may display a first guide object in operation 2720.

Referring to FIG. 28, a first guide object 1621a for guiding a photographing position is illustrated. As illustrated, when the electronic device 130 and the in vitro diagnostic device 110 are disposed such that the photographing angle (e.g., the photographing angle between the electronic device 130 and the in vitro diagnostic device 110) of the in vitro diagnostic device 110 satisfies a preset angle (e.g., 10 degrees) or a preset angle range (e.g., 5 to 30 degrees), at least a portion (e.g., the pad 2503) of the in vitro diagnostic device 110 may be displayed in a trapezoidal shape that is close to a rectangular shape on a preview image including the in vitro diagnostic device 110. The electronic device 130 may guide not only a photographing position but also a photographing angle (e.g., a relative inclination) of the in vitro diagnostic device 110 through the first guide object 1621a. As an example, as illustrated in FIG. 28(a), the first guide object 1621a may be configured in a rectangular shape to correspond to the shape of the pad 2503. As another example, as illustrated in FIG. 28(b), the first guide object 1621a may be configured in a shape that is associated with a photographing angle (e.g., a photographing angle between the electronic device 130 and the in vitro diagnostic device 110). In more detail, the first guide object 1621a may be configured in a shape (e.g., a trapezoidal shape that is close to a rectangular shape) that is expected when a photographing angle (e.g., a photographing angle between the electronic device 130 and the in vitro diagnostic device 110) of the in vitro diagnostic device 110 satisfies a preset angle (e.g., 10 degrees) or a preset angle range (e.g., 5 to 30 degrees).

According to various embodiments, the electronic device 130 may identify whether the photographing inclination condition is satisfied based on information that is acquired from the inertia sensor in operation 2730. For example, the electronic device 130 may identify whether the inclination (e.g., the absolute inclination) of electronic device 130 satisfies a preset angle (e.g., 10 degrees) or a preset angle range (e.g., 5 to 30 degrees) based on sensing values that are acquired from at least one inertia sensor. According to various embodiments, when it is identified that the photographing inclination condition is not satisfied, the electronic device 130 may repeat operation 2730.

According to various embodiments, when it is identified that the photographing inclination condition is satisfied, the electronic device 130 may identify whether a degree of similarity of at least a portion of the in vitro diagnostic device 110 to the first guide object is included within a specific range.

For example, as illustrated in FIG. 28(a), when the first guide object 1621a is configured in a rectangular shape corresponding to the shape of the pad 2503, a specific range for including a degree of similarity between at least a portion of the in vitro diagnostic device 110 and the first guide object 1621a may be set to a first degree to a second degree. In this case, the second degree (i.e., the upper limit range) may be set in association with a preset angle (e.g., 10 degrees) or a preset angle range (e.g., 5 to 30 degrees). In more detail, while the first guide object 1621a is set to be rectangular, the shape of the pad 2503 that is expected while the inclination (e.g., the relative inclination) of the electronic device 130 satisfies a preset angle (e.g., 10 degrees) or a preset angle range (e.g., 5 to 30 degrees) may be a trapezoidal shape that is close to a rectangle. When the pad 2503 in the first guide object 1621a is displayed on the preview image while the inclination (e.g., the absolute inclination) of the electronic device 130 satisfies a preset angle (e.g., 10 degrees) or a preset angle range (e.g., 5 to 30 degrees), the electronic device 130 may identify whether the area of the pad 2503 compared to the area of the first guide object 1621a is equal to or less than a specific value (e.g., 80%), and/or whether the angle of the left/right side of the pad 2503 with respect to the left/right side of the first guide object 1621a is equal to or greater than a specific value (e.g., 10 degrees).

As another example, as illustrated in FIG. 28(b), when the first guide object 1621a is configured in a shape (e.g., a trapezoidal shape close to a rectangular) that is associated with the photographing angle (e.g., the photographing angle between the electronic device 130 and the in vitro diagnostic device 110), a specific range, in which a degree of similarity between at least a part of the in vitro diagnostic device 110 and the first guide object 1621a is to be included, may be set to the first degree or more. Then, the first degree (i.e., the lower limit range) may be set to be substantially close to 100% (e.g., 90%). Even in this case, when the pad 2503 in the first guide object 1621a is displayed on the preview image while the inclination (e.g., the absolute inclination) of the electronic device 130 satisfies a preset angle (e.g., 10 degrees) or a preset angle range (e.g., 5 to 30 degrees), the electronic device 130 may identify whether the area of the pad 2503 compared to the area of the first guide object 1621a is equal to or more than a specific value (e.g., 90%), and/or whether the angle of the left/right side of the pad 2503 with respect to the left/right side of the first guide object 1621a is equal to or less than a specific value (e.g., 5 degrees).

According to various embodiments, the electronic device 130 may perform the first function in operation 2750 when it is identified that the degree of similarity of at least a portion of the in vitro diagnostic device 110 to the first guide object is included in a specific reference. For example, the first function may include enabling of the photographing object 1623, execution of a photographing function, and/or execution of a flash function of the camera.

According to various embodiments, the electronic device 130 may perform the second function in operation 2760 when it is not identified that the degree of similarity of at least a portion of the in vitro diagnostic device 110 to the first guide object is included in a specific reference. For example, the second function may include disenabling of the photographing object 1623 and/or display of a notification message (e.g., a message that indicates that photographing is impossible). According to an embodiment, the electronic device 130 may repeat operation 2740 when it is not confirmed that at least a part of the in vitro diagnostic device 110 includes a degree similar to the first guide object within a specific range.

According to various embodiments, when it is not identified that a degree of similarity of at least a portion of the in vitro diagnostic device 110 to the first guide object is not included within a specific range, the second function may be performed in operation 2760.

Hereinafter, examples of operations of the electronic device 130 according to various embodiments will be further described.

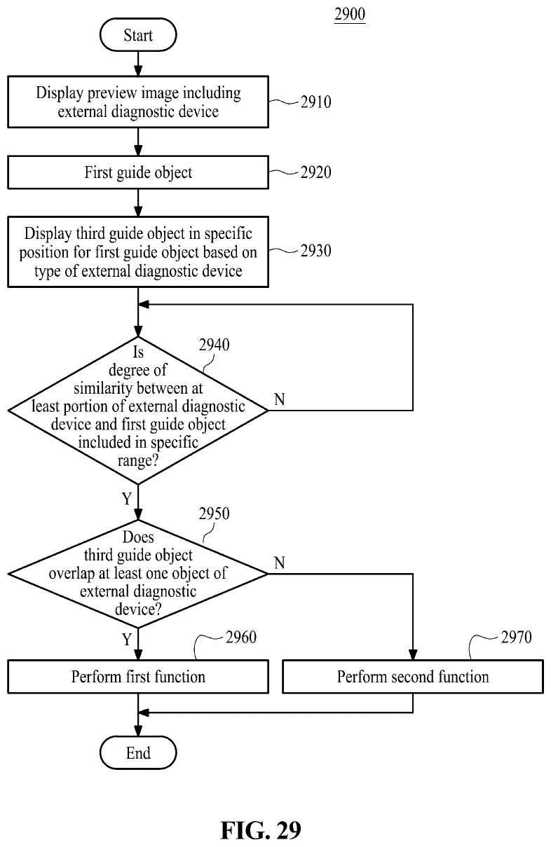

FIG. 29 is a flowchart 2900 illustrating a method of guiding a disposition direction of the in vitro diagnostic device 110 by the electronic device 130 (e.g., processor 1409), according to various embodiments. According to various embodiments, the operations illustrated in FIG. 29 are not limited to the order illustrated, and may be performed in various orders. Furthermore, according to various embodiments, at least one operation that is less or more than the operations illustrated in FIG. 29 may be performed.

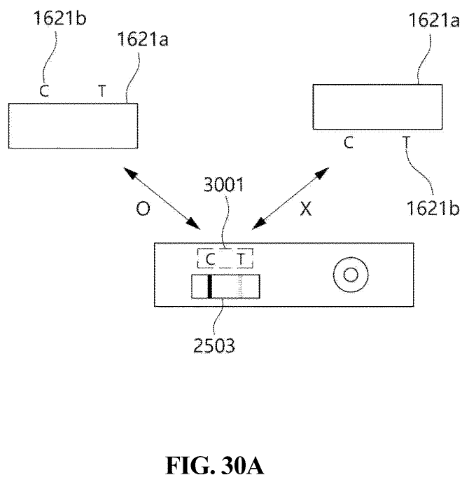

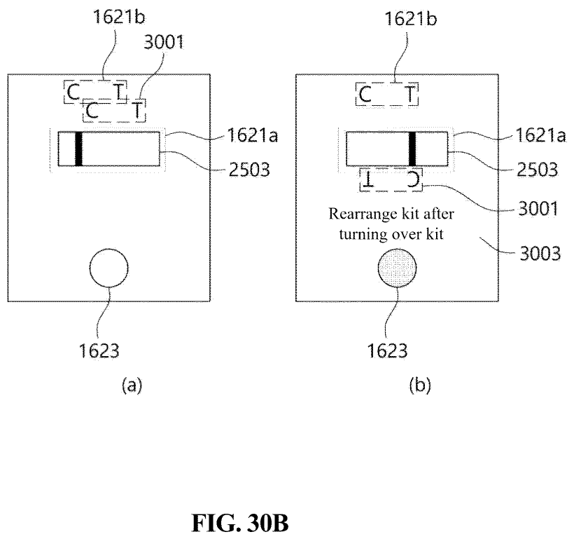

FIG. 30A is a view illustrating a method of guiding a disposition direction of the in vitro diagnostic device 110 by the electronic device 130 (e.g., processor 1409), according to various embodiments. FIG. 30B illustrates a screen for guiding a disposition direction of the in vitro diagnostic device 110 by the electronic device 130 (e.g., the processor 1409), according to various embodiments.

According to various embodiments, the electronic device 130 may display a preview image including the in vitro diagnostic device 110 in operation 2910.

According to various embodiments, the electronic device 130 may display a first guide object in operation 2920. For example, as illustrated in FIG. 28, the first guide object 1621a may be configured (e.g., in a rectangular shape) to correspond to the shape of the pad 2503, and/or may be configured in a shape (e.g., a trapezoidal shape that is close to a rectangular shape) that is expected when the photographing angle (e.g., the photographing angle between the electronic device 130 and the in vitro diagnostic device 110) satisfies a preset angle (e.g., 10 degrees) or a preset angle range (e.g., 5 to 30 degrees).

According to various embodiments, the electronic device 130 may display the third guide object 1621b at a specific position for the first guide object 1621a based on the type of the in vitro diagnostic device 110 in operation 2930. For example, the position, in which the third guide object 1621b is displayed, may be determined based on information on information on various components (e.g., the pad 220 of FIG. 2A) that constitute the in vitro diagnostic device 110, a reaction area (e.g., the test area 211 of FIG. 2A and the control area 213 of FIG. 2A), information on surrounding objects (e.g., a specific patterns and/or engraving, and the like.), and/or at least one guide object, which is included in the product information of the in vitro diagnostic device 100 and/or identified based on the product information. For example, as illustrated in FIG. 30A, at least one object (e.g., a text of “C” and/or “T”) 3001 may be included on the housing (e.g., the housing 210 of FIG. 2A) of the in vitro diagnostic device 110 within a specified distance from a specific portion (e.g., the pad 2503 (e.g., the pad 220 of FIG. 2A)) of the in vitro diagnostic device 110. Each object (e.g., the text of “C” and/or “T”) may indicate each inspection line (e.g., the test line and/or the control line) that appears in the pad 2503. As another example, at least one object 3001 may be configured in the form of a specific pattern and/or engraving (e.g., an embossed part and/or an engraved part) within a specified distance from a specific portion (e.g., the pad 2503) of the in vitro diagnostic device 110 on the housing (e.g., the housing 210 of FIG. 2A) of the in vitro diagnostic device 110. At least one object 3001 may be located in a first position (e.g., an upper position) with respect to a specific portion (e.g., the pad 2503) depending on the type of in vitro diagnostic device 110, as illustrated in FIG. 30A. Alternatively, the at least one object 3001 may be located in a second position (e.g., a lower position) with respect to the specific portion (e.g., the pad 2503) depending on the type of in vitro diagnostic device 110, or may be located in various other positions (e.g., a left or right position). According to various embodiments, the electronic device 130 may identify position information of the at least one object 3001 depending on the type of in vitro diagnostic device 110 and display a third guide object 1621b such that it corresponds to the identified position information. Referring back to FIG. 30A, when at least one object 3001 of the in vitro diagnostic device 110 is a device that is located on an upper side of a specific portion (e.g., the pad 2503), the electronic device 130 may display the third guide object 1621b on an upper side of the first guide object 1621a.

According to various embodiments, the electronic device 130 may identify whether a degree of similarity of at least a portion of the in vitro diagnostic device 110 to the first guide object is included within a specific range in operation 2940. According to an embodiment, the electronic device 130 may perform operation 2940 when it is identified that the photographing inclination condition is satisfied after performing operation 2730 of FIG. 27, before performing operation 2940. According to various embodiments, the electronic device 130 may repeat operation 2940 when it is not identified that the degree of similarity of at least a portion of the in vitro diagnostic device 110 to the first guide object is included in a specific reference. According to an embodiment, the electronic device 130 may perform the second function when it is not identified that the degree of similarity of at least a portion of the in vitro diagnostic device 110 to the first guide object is included in a specific reference.

According to an embodiment, when it is identified that the degree of similarity of at least a portion of the in vitro diagnostic device 110 to the first object is included within a specific range, the electronic device 130 may identify whether the third object overlaps at least one object of the in vitro diagnostic device 110 in operation 2950. For example, referring to FIG. 30B, a preview image 2505 including at least a portion e.g., (a membrane 2503), and at least one object 3001 of the in vitro diagnostic device 110 may be displayed through the guide screen 1620, and a first guide object 1621a, a third guide object 1621b, and a photographing object 1623 may be displayed together with the preview image 2505. A relative position between the pad 2503 and the first guide object 1621a and a relative position between at least one object 3001 and the third guide object 1621b may be changed according to the movement of the electronic device 130.

According to various embodiments, the electronic device 130 may perform the first function in operation 2960 when the third guide object overlaps at least one object of the in vitro diagnostic device 110. For example, referring to FIG. 30B, according to the movement of the electronic device 130, the pad 2503 and the first guide object 1621a may overlap each other, and while the pad 2503 and the first guide object 1621a overlap each other, the electronic device 130 may perform the first function when at least one object 3001 and the third guide object 1621b overlap each other. For example, the first function may include enabling of the photographing object 1623, execution of a photographing function, and/or execution of a flash function of the camera 1401.

According to various embodiments, the electronic device 130 may perform the second function in operation 2970 when the third guide object does not overlap at least one object of the in vitro diagnostic device 110. For example, referring to (b) of FIG. 30B, when the in vitro diagnostic device 110 is arranged upside down (e.g., turned over) when the in vitro diagnostic device 110 includes at least one object 3001 in the first position (e.g., an upper position) with respect to the pad 2503, at least one object 3001 of the in vitro diagnostic device 110 and the third guide object 1621b may not overlap each other. In the electronic device 130, when the third guide object 3001 does not overlap at least one object of the in vitro diagnostic device 110, the photographing object 1623 may be disenabled, and/or a notification message (e.g., “Rearrange the kit by turning over the kit”) (3003) may be displayed. According to an embodiment, the electronic device 130 may perform the first function when at least one object 3001 and the third guide object 1621b overlap each other as the disposition direction of the in vitro diagnostic device 110 is changed (e.g., aligned after overturning).

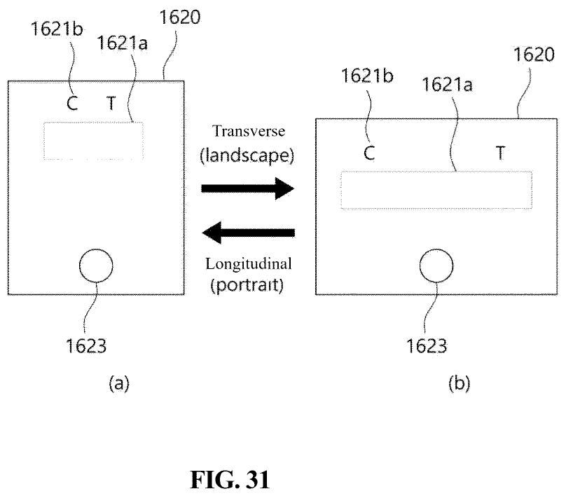

FIG. 31 illustrates examples of at least one object (e.g., 1621a, and/or 1621b) that is displayed based on an disposition direction of the electronic device 130 according to various embodiments.

Referring to FIG. 31, a preview image (not illustrated), at least one guide object (e.g., a first guide object 1621a, a second guide object (not illustrated), and/or a third guide object 1621b) and/or a photographing object 1623 may be displayed on the guide screen 1620.

According to various embodiments, the display state of the preview image (not illustrated), at least one guide object (e.g., the first guide object 1621a, the second guide object 1621b, and/or the third guide object 1621b), and/or the photographing object 1623 may be changed depending on the direction, in which the electronic device 130 is disposed. According to various embodiments, the direction, in which the electronic device 130 is disposed, may be identified based on sensing values that are acquired from at least one sensor 1405 (e.g., the inertia sensor) of the electronic device 130.

For example, when the electronic device 130 is disposed in the first direction (e.g., the lengthwise direction), at least one guide object (e.g., the first guide object 1621a, the second guide object (not illustrated), and/or the third guide object 1621b) may be displayed in the first size, as illustrated in FIG. 31(a).

For example, when the electronic device 130 is disposed in a second direction (e.g., the horizontal direction) that is perpendicular to the first direction, at least one guide object (e.g., the first guide object 1621a, the second guide object (not illustrated), and/or the third guide object 1621b) may be displayed in a second size that is larger than the first size.

According to various embodiments, when the electronic device 130 is rotated in the second direction while being disposed in the first direction, at least one guide object (e.g., the first guide object 1621a, the second guide object (not illustrated), and/or the third guide object 1621b) may be enlarged to be displayed in the second size. On the contrary, when the electronic device 130 is rotated in the first direction while being disposed in the second direction, at least one guide object (e.g., the first guide object 1621a, the second guide object (not illustrated), and/or the third guide object 1621b) may be reduced to be displayed in the first size.

According to various embodiments, the photographing object 1623 may be displayed in the same size regardless of the disposition direction of the electronic device 130. According to an embodiment, when the electronic device 130 is rotated from the first direction to the second direction, it is to be enlarged to be displayed, and when the electronic device 130 is rotated from the second direction to the first direction, it may be reduced to be displayed

Hereinafter, examples of operations of the electronic device 130 according to various embodiments will be further described.

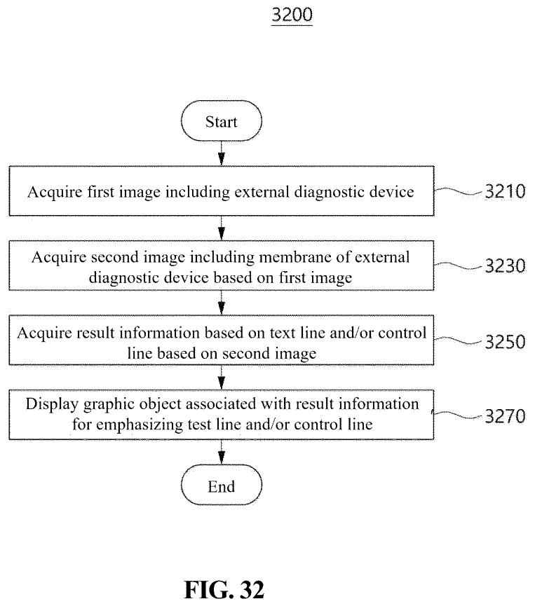

FIG. 32 is a flowchart 3200 illustrating a method of providing result information based on at least one inspection line (e.g., a test line, and/or a control line) by the electronic device 130 (e.g., the processor 1409) according to various embodiments. According to various embodiments, the operations illustrated in FIG. 32 are not limited to the order illustrated, and may be performed in various orders. Furthermore, according to various embodiments, at least one operation that is less or more than the operations illustrated in FIG. 32 may be performed.

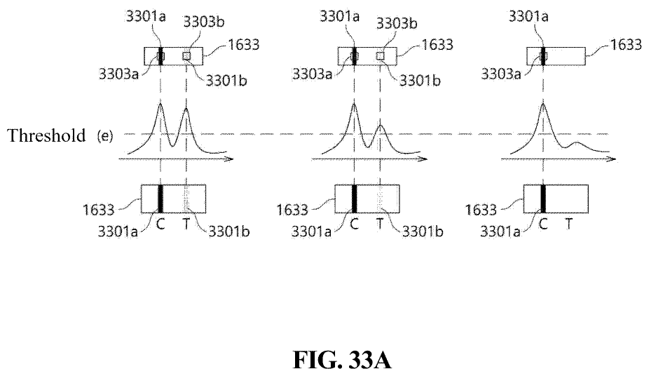

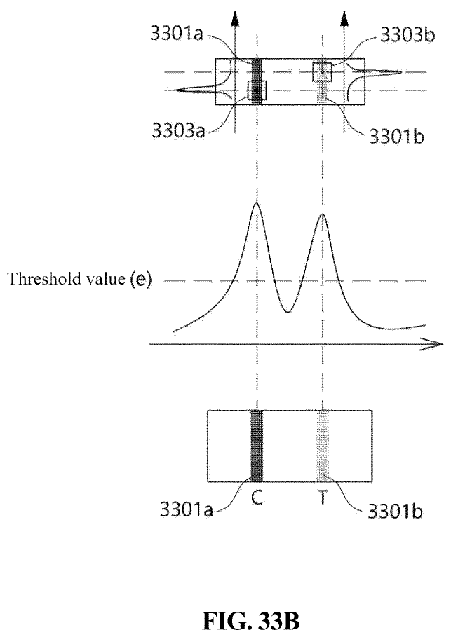

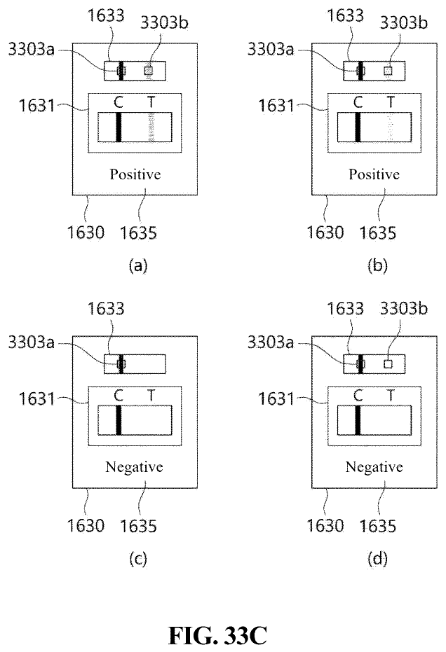

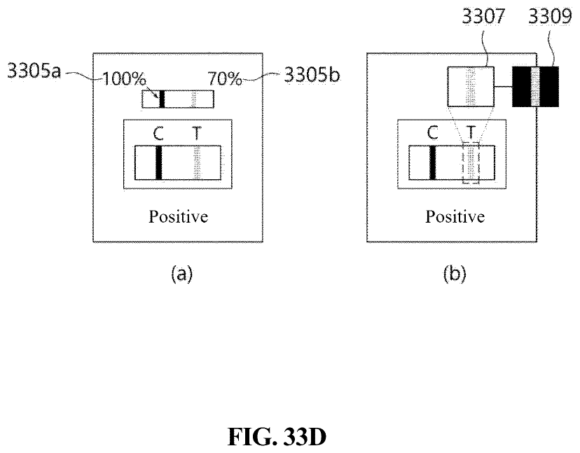

FIGS. 33A and 33B are views illustrating a method of acquiring result information based on at least one inspection line (e.g., a test line, and/or a control line) by the electronic device 130 according to various embodiments. FIG. 3C is screens that provide result information based on at least one inspection line (e.g., a test line, and/or a control line) by the electronic device 130 (e.g., the processor 1409) according to various embodiments. FIG. 33D illustrates another example of a graphic object that emphasizes at least one inspection line (e.g., a test line and/or a control line), according to various embodiments.

According to various embodiments, the electronic device 130 may acquire a first image including the in vitro diagnostic device 110 in operation 3210. For example, the electronic device 130 may acquire a first image (e.g., the first image 1631 of FIG. 16) including the in vitro diagnostic device 110 by photographing the in vitro diagnostic device 110 including at least one pad (e.g., the pad 2503 of FIG. 25). According to an embodiment, the electronic device 130 may acquire the first image photographed while the photographing inclination (e.g., the photographing angle) and/or the photographing position condition described above is satisfied.

According to various embodiments, the electronic device 130 may acquire a second image including the pad 2523 (or for the pad 2503) of the in vitro diagnostic device 110 based on the first image. For example, the electronic device 130 may identify the position of the pad 2503 in the first image, perform at least one image processing for the first image, and acquire a second image (e.g., the second image 1633 of FIG. 16) including the pad 2503. As an example, when a foreign object is included on the pad 2503 included in the first image, the electronic device 130 may acquire a second image, from which the foreign object has been removed, by performing image processing for removing the foreign object from the first image. According to various embodiments, the electronic device 130 may acquire result information based on the test line and/or the control line based on the second image in operation 3250. Meanwhile, without being limited to the described example, the second image (e.g., the second image 1633 of FIG. 16) may be used for the purpose of providing a reliability for the result, and the electronic device 130 may acquire result information based on the test line and/or the control line based on the first image and/or the corrected first image.

According to various embodiments, the electronic device 130 may identify biometric measurement information (e.g., an amount and/or a concentration of antibodies in the case of an antigen diagnosis) that is to be inspected by the in vitro diagnostic device 110 based on the color value (e.g., R, G, and B values, or CYMK values) of the test line identified from the first image and/or the second image, and may acquire an in vitro diagnosis result based on the biometric information. Then, the electronic device 130 may correct the biometric information based on a quantitative value and/or a surrounding environment information (e.g., a surrounding temperature) of the in vitro diagnostic device 110 as described above, as at least a portion of an operation of calculating the amount and/or the concentration of the amount and/or the concentration of the biometric information measurement value (e.g., the antibody, in the case of the antigen diagnosis method). Furthermore, the electronic device 130 may determine information (e.g., a tertiary regression function) for measuring the biometric information measurement value corresponding to the photographing environment value (e.g., a brightness value) identified based on at least one sign 230 (e.g., determine the coefficient of the function, and/or select a corresponding one of a plurality of functions itself, and may identify (e.g., based on the result values of inputting each of the R, G, and B values into the function) the biometric information measurement value (e.g., a concentration of an antigen or an antibody).

Referring to FIGS. 33A and/or 33B, the electronic device 130 may identify analysis information of the test line 3301b and/or the control line 3301a. For example, the analysis information may include a color value and/or a color concentration of at least one inspection line. For example, the color value is a numerical value that is obtained by converting a color feature into a coordinate value of a geometric color space, and may include a red, green, and blue (RGB) value or a cyan, magenta, yellow, and (black) ke (CMYK) value. For example, the color concentration may include a tone, such as a brightness and/or a chroma (saturation). According to various embodiments, the color concentration illustrated in the test line 3301b and/or the control line 3301a may vary depending on the concentration of the measurement object (e.g., a reagent, and/or an antigen). For example, the electronic device 130 may determine whether the test line 3301b and/or the control line 3301a is effective based on the color concentration illustrated in the test line 3301b and/or the control line 3301a. For example, referring to FIG. 33A, a threshold value “e” is described. The threshold value “e” may be set to correspond to a limit of detection (LOD) value range of 190 to 210 that is associated with the in vitro diagnostic device 110, but the above-described LOD value range may be set to a different value range. The electronic device 130 may identify that the test line 3301b is effective when the concentration of color illustrated on the test line 3301b is more than the threshold value “e”. The electronic device 130 may identify that the control line 3301a is effective when the concentration of color illustrated on the control line 3301a is more than the threshold value “e”. For convenience of description, the threshold value “e” for the test line 3301b and the threshold value “e” for the control line 3301a are illustrated as the same value, but the threshold value “e” for the test line 3301b and the threshold value “e” for the control line 3301a may be set to different values. According to an embodiment, when the in vitro diagnostic device 110 includes at least one sign 230, the electronic device 130 may identify that the test line 3301b is effective when the color tone of the color illustrated in the test line 3301b is more than the threshold value while the test line 3301b has color values included in at least one sign 230. As another example, when the in vitro diagnostic device 110 does not include at least one sign 230, product information on various components (e.g., the pad 210 of FIG. 2A) that constitute the in vitro diagnostic device 110, the reaction areas (e.g., the test area 211 of FIG. 2A, and the control area 213 of FIG. 2A) of the in vitro diagnostic device 110, and information on surrounding objects (e.g., specific patterns and/or engraving) may be identified, and color values which the control line 3301a and/or the test line 3301b may have may be identified based on the product information, and when the concentration of the color illustrated in the control line 3301a and/or the test line 3301b is more than the threshold “e” while the control line 3301a and/or the test line 3301b has identified color values, it may be confirmed that the control line 3301a and/or the test line 3301b are effective. According to various embodiments, the electronic device 130 may display a result screen 1630 including diagnosis result information that is identified based on result information including whether the test line 3301b and/or the control line 3301a is effective, and this will be described in more detail with reference to the drawings described later.

According to various embodiments, the electronic device 130 may display a graphic object that is associated with result information for emphasizing a test line and/or a control line in operation 3270.

Referring to FIG. 33A, the electronic device 130 may display, for the test line 3301b and/or the control line 3301a, a graphic object that emphasizes a portion of each color value, in which the concentration of the color is more than the threshold value “e”. For example, referring to FIG. 33A, when there is a point, at which the concentration of the color is more than the threshold “e” in the area, in which the control line 3301a appears in the second image 1633 (or when the average concentration value of the color of the area, in which the control line 3301a appears, is more than the threshold “e”, the electronic device 130 may display a graphic object 3303a for an area, in which the control line 3301a appears in the second image 1633. In this case, when there is no point in the area of the control line 3301a in the second image 1633, at which the concentration of the color is more than the threshold “e” (or when the average concentration value of the color of the area, in which the control line 3301a appears, is more than the threshold “e”, the electronic device 130 may not display the graphic object 3303a (or refrain the display). According to an embodiment, when there is no point in the area of the control line 3301a in the second image 163, at which the concentration of the color is more than the threshold “e” (or when the average concentration value of the color of the area, in which the control line 3301a appears, is not more than the threshold “e”, the electronic device 130 may display an imaginary control line (e.g., in the form of a dotted line) in an area of the control line 3301a included in the second image 1633. According to various embodiments, when there is a point, at which the concentration of the color is more than the threshold “e” in the area, in which the test line 3301b appears in the second image 1633 (or when the average concentration value of the color of the area, in which the test line 3301b appears, is more than the threshold “e”, the electronic device 130 may display a graphic object 3303a for an area, in which the control line 3301a appears in the second image 1633. In this case, when there is no point in the area of the test line 3301b in the second image 1633, at which the concentration of the color is more than the threshold “e” (or when the average concentration value of the color of the area, in which the test line 3301b appears, is more than the threshold “e”, the electronic device 130 may not display the graphic object 3303b (or refrain the display). According to an embodiment, when there is no point in the area of the test line 3301b in the second image 163, at which the concentration of the color is more than the threshold “e” (or when the average concentration value of the color of the area, in which the test line 3301b appears, is not more than the threshold “e”, the electronic device 130 may display an imaginary test line (e.g., in the form of a dotted line) in an area of the test line 3301b included in the second image 1633. According to various embodiments, the electronic device 130 may display a graphic object 3301a and/or 3301b for a point, at which the color concentration is the highest, in the areas, in which the control line 3301a and/or the test line 3301b appears. For example, referring to FIG. 33b, the electronic device 130 may display a graphic object 3303a for a point (e.g., one point in the longitudinal direction of the area, in which the control line 3301a appears), at which where the concentration of the color is more than the threshold “e” and has the highest color concentration in the areas, in which the control line 3301a appears in the second image 1633. The electronic device 130 may display a graphic object 3303b for a point (e.g., one point in the longitudinal direction of the area, in which the control line 3301a appears), at which the concentration of the color is more than the threshold “e” and has the highest color concentration in areas, in which the test line 3301b appears in the second image 1633. According to an embodiment, even when there is no point in the area of the control line 3301a (or the test line 3301b) in the second image 1633, at which the concentration of the color is more than the threshold value “e”, the electronic device 130 may display a graphic object 3303a or a graphic object 3303b for a point has the highest color concentration in areas of the control line 3301a (or the test line 3301b).

According to various embodiments, the electronic device 130 may display the graphic object 3303a and/or 3303b described above through the result screen 1630 together with the diagnosis result information that is identified based on the test line 3301b and/or the control line 3301a.

Referring to FIGS. 33C and 33D, result screens 1630 including various diagnosis result information are illustrated. The result screen 1630 may include a first image 1631, a second image 1633, and an object 1635 that indicates result information. The second image 1633 may be acquired from the first image 1631, and may be displayed in an area that is adjacent to the first image 1631. A graphic object 3303a and/or 3303b may be displayed on the second image 1633. As described above, the graphic object 3303a and/or 3303b may be displayed in an area or point, at which the color concentration is more than the threshold value e or the color concentration is highest. Referring to FIGS. 33C(a) and 33C(b), the electronic device 130 may display an object 1635 that indicates a diagnosis result of ‘positive’ based on that both the control line 3301a and the test line 3301b are effective. When FIG. 33C(a) and FIG. 33C(b) are compared, the color concentration of the test line 3301b of FIG. 33C(a) is greater than that of the test line 3301b of FIG. 33C(b). In this case, the electronic device 130 may identify that the color concentration of the test line 3301b of (b) is more than the threshold value “e”, and as a result, may identify a diagnosis result of ‘negative’ even in the case of (b). Referring to FIGS. 33C(c) and 33B(d), the electronic device 130 may display an object 1635 that indicates a diagnosis result of ‘positive’ based on that both the control line 3301a is effective but the test line 3301b is not effective. The electronic device 130 may display a graphic object 3301a for emphasizing the control line 3301a based on the fact that the control line 3301a is effective but the test line 3301b is not effective. Referring to FIG. 33C(c), the electronic device 130 may not display the graphic object 3303b that emphasizes the test line 3301b based on that the test line 3301b is not effective. Referring to FIG. 33C(c), the electronic device 130 may not display the graphic object 3303b to emphasize an area or a point, at which the color concentration is highest, among the areas, in which the test line 3301b appears, even when the test line 3301b is not effective. Referring to FIG. 33D(a), the graphic object that emphasizes at least one test line (e.g., the control line 3301a, and/or the test line 3301b) may be configured in the form of values 3305a and 3305b (e.g., 100%, 70%, or the like) that indicates a color concentration. Referring to FIG. 33D(b), when a specific line, among the control line 3301a and the test line 3301b included in the second image 1633, is selected, an image 3307, in which the selected specific line is enlarged, may be displayed. In this case, the electronic device 130 may perform at least one image processing (e.g., color correction) on an image 3307 of the second image 1633, in which a selected specific line is enlarged, and display an image-processed enlarged image 3309.

Hereinafter, examples of operations of the electronic device 130 according to various embodiments will be further described.

According to various embodiments, when a specified photographing condition (e.g., a photographing inclination) is satisfied, the electronic device 130 may acquire an image of the in vitro diagnostic device 110 by performing flash photographing.

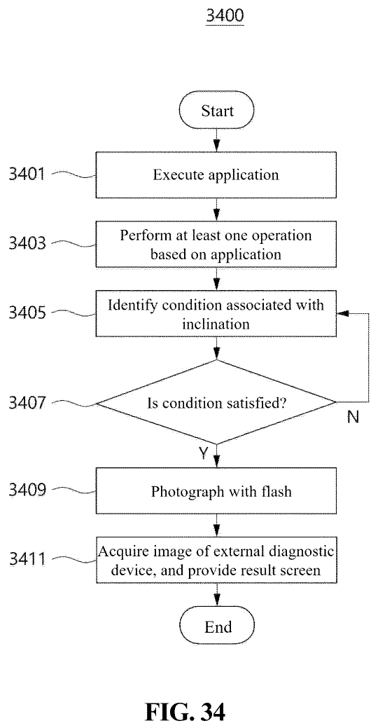

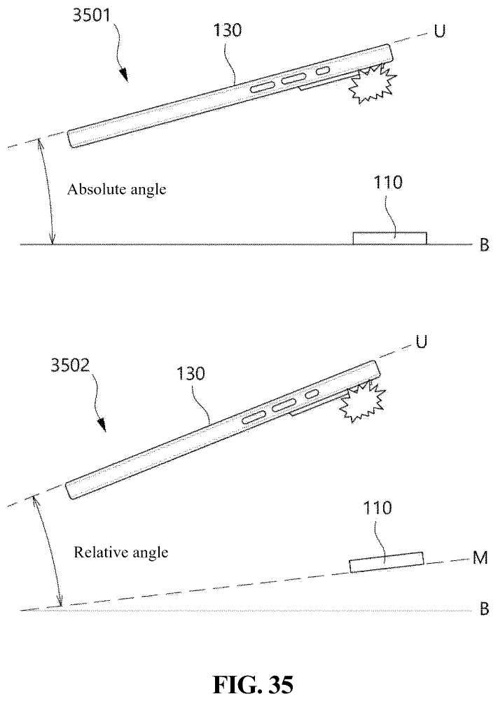

FIG. 34 is a flowchart 3400 illustrating an example of an operation of the electronic device 130 according to various embodiments. According to various embodiments, the operations illustrated in FIG. 34 are not limited to the order illustrated, and may be performed in various orders. Furthermore, according to various embodiments, at least one operation that is less or more than the operations illustrated in FIG. 34 may be performed. Hereinafter, FIG. 34 will be further described with reference to FIG. 35.

FIG. 35 is a view illustrating an example of an absolute angle and a relative angle according to various embodiments.

According to various embodiments, the electronic device 130 (e.g., the processor 1409) may execute an application in operation 3401, and may perform at least one operation based on the application in operation 3403. For example, the electronic device 130 may perform an operation of providing at least one execution screen (e.g., a timer UI (e.g., the timer screen 1610 of FIG. 16), a guide UI (e.g., the guide screen 1620 of FIG. 16), and/or a result screen UI (e.g., the result screen 1630 of FIG. 16) based on execution of an application that is configured to provide the above-described in vitro diagnosis result, and/or providing at least one function based on the provided at least one execution screen.

According to various embodiments, the electronic device 130 (e.g., the processor 1409) may identify a condition that is associated with the inclination in operation 3405, and may determine whether the condition associated with the inclination is satisfied in operation 3407. For example, while displaying the above-described guide UI, the electronic device 130 may identify a condition (e.g., the absolute angle and/or the relative angle) that is associated with the inclination as illustrated in FIG. 35, and may determine whether the condition associated with the identified inclination satisfies the specified condition. Satisfying the specified condition may mean that the angle satisfies a range of 10 degrees to 20 degrees, and/or more specifically, may mean that a specific angle (e.g., 15 degrees) is satisfied.

According to various embodiments, the electronic device 130 (e.g., the processor 1409) may sense (or acquire) an angle (e.g., the absolute angle) between the ground “B” and a surface “U” (or an axis) of the electronic device 130 in the lengthwise direction as illustrated in FIG. 351 by using the sensor 1405 (e.g., the inertia sensor, the inclination sensor, and the angular velocity sensor) provided in the electronic device 130.

According to various embodiments, the electronic device 130 (e.g., the processor 1409) may sense (or acquire) an angle (a relative angle) between a surface “M”, on which the in vitro diagnostic device 110 is disposed, and the surface “U” (or an axis) of the electronic device 130 in the lengthwise direction as illustrated in 3501 of FIG. 35, based on an image of the in vitro diagnostic device 110, which is captured by using the camera. As an object that obstructs the disposition of the in vitro diagnostic device 110 is located on the ground “B”, and/or a physical structure is formed thereon, the lower surface of the in vitro diagnostic device 110 may not be disposed to contact the ground “B”. Accordingly, the electronic device 130 may photograph the in vitro diagnostic device 110 while an appropriate photographing condition is not satisfied. Accordingly, to solve the above problem, the electronic device 130 may acquire a relative angle based on the image of the in vitro diagnostic device 110, which is acquired by using the camera 1401 internally while displaying the guide UI.

For example, the electronic device 130 may store information on the relative angle for each feature of the in vitro diagnostic device 110 that is detectable from the image in the memory 1411 in advance, and may acquire the relative angle based on a comparison between the pre-stored information and the specifications identified from the image of the in vitro diagnostic device 110. The features of the in vitro diagnostic device 110 may include an attribute (e.g., a position, a number) of the feature points identified from the image, at least some components (e.g., the pad 220 or the hole 211) of the in vitro diagnostic device 110, and/or the feature point (e.g., a position or a shape) of the physical structure (e.g., the inclination surface), and/or the features (e.g., a position or a shape) of the visual marker. For example, after performing an experiment, in which information on features of the in vitro diagnostic device 110 identified from an image of the in vitro diagnostic device 110 is identified in advance by the relative angle between the electronic device 130 and the in vitro diagnostic device 110 is changed, information on features for each of the relative angles may be acquired and be stored in the electronic device 130 in advance. Alternatively, the information may be stored in the server 140 and provided to the electronic device 130.

As another example, the electronic device 130 may store information on the reference image of the in vitro diagnostic device 110 corresponding to a specific angle (e.g., 15 degrees) in advance, and may acquire a relative angle based on the degree of similarity, which is identified based on a comparison between the reference image and the currently acquired image of the in vitro diagnostic device 110. For example, when the degree of similarity is low and the difference is identified, the electronic device 130 may acquire a relative angle by subtracting and/or adding the difference identified from a specific angle (e.g., 15 degrees).

Meanwhile, according to various embodiments, operation 3409 may be performed when the condition is satisfied as one of the operation of sensing the absolute angle and the operation of sensing the relative angle of the electronic device 130 is performed, but operation 3409 may be performed when each condition is satisfied as both the absolute angle and the relative angle are performed.

According to various embodiments, when the condition associated with the inclination is satisfied (3407—Y), the electronic device 130 (e.g., the processor 1409) may perform flash photographing in operation 3409, may acquire an image of the in vitro diagnostic device 110 in operation 3411, and may provide a result screen. For example, when information (e.g., the absolute angle, and/or the relative angle) associated with the identified inclination satisfies a specified condition (e.g., satisfies an angle range), the electronic device 130 may perform photographing by using the camera 1401 in a state of a flash (not illustrated) is enabled. Accordingly, the amount of light generated by the flash is reflected by the in vitro diagnostic device 110 and the amount of the light input to the electronic device 130 is remarkably reduced, and thus an image of the in vitro diagnostic device 110 may be easily analyzed.

Hereinafter, examples of operations of the electronic device 130 according to various embodiments will be further described.

According to various embodiments, the electronic device 130 may identify the disposition state of the in vitro diagnostic device 110 based on the image of the in vitro diagnostic device 110, and may select the type information (e.g., the absolute angle and the relative angle) of associated with the inclination, by which it is to be determined whether the condition is satisfied based on the identified disposition state.

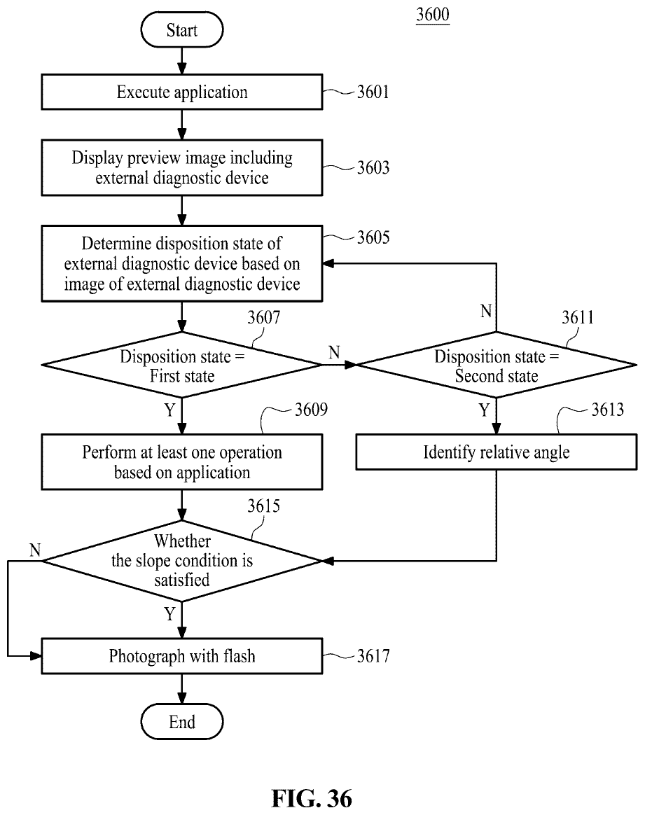

FIG. 36 is a flowchart 3600 illustrating an example of an operation of the electronic device 130 according to various embodiments. According to various embodiments, the operations illustrated in FIG. 36 are not limited to the order illustrated, and may be performed in various orders. Furthermore, according to various embodiments, at least one operation that is less or more than the operations illustrated in FIG. 36 may be performed.

According to various embodiments, the electronic device 130 (e.g., the processor 1409) may execute an application in operation 3601, and may perform at least one operation based on the application in operation 3603. For example, operations 3601 to 3603 of the electronic device 130 may be performed in the same manner as operations 3401 to 3403 described above, and thus a repeated description thereof will be omitted.

According to various embodiments, the electronic device 130 (e.g., the processor 1409) may determine the disposition state of the in vitro diagnostic device 110 based on the image of the in vitro diagnostic device 110 in operation 3605. For example, the electronic device 130 may determine the disposition state of the in vitro diagnostic device 110 based on an image including the in vitro diagnostic device 110 acquired by using the camera 1401 while the guide UI is displayed. For example, the disposition state may include a first state (or a normal state) that is a state, in which the lower surface of the in vitro diagnostic device 110 is disposed to be adhered to the ground, and a second state (or an abnormal state) that is a state (or an inclined state), in which the lower surface of the in vitro diagnostic device 110 is not adhered thereto, but is not limited to the examples described. For example, the electronic device 130 may determine the disposition state of the in vitro diagnostic device 110 by comparing the pre-stored reference information with the image of the in vitro diagnostic device 110. The reference information may be information on features (e.g., feature points, a configuration/physical structure, and visual markers) of the in vitro diagnostic device 110 when the in vitro diagnostic device 110 is disposed in a normal state. As a result of comparing the reference information with an image of the in vitro diagnostic device 110, the electronic device 130 may determine the result as an abnormal state when the degree of similarity is less than a threshold value, and may determine the result as a normal state when the degree of similarity is equal to or greater than the threshold value. To the contrary, the reference information may be information on features (e.g., feature points, a configuration/physical structure, and visual markers) of the in vitro diagnostic device 110 when the in vitro diagnostic device 110 is disposed in an abnormal state. Accordingly, when the degree of similarity identified as a result of the comparison is equal to or greater than a threshold value, it may be determined as an abnormal state, and may also be determined as a normal state when the degree of similarity is equal to or less than the threshold value.

According to various embodiments, the electronic device 130 (e.g., the processor 1409) may determine whether the determined disposition state is a first state (e.g., a normal state) in operation 3607, and may determine an absolute angle in operation 3609 in the first state (e.g., the normal state) (3607—Y), and may identify a relative angle in operation 3613 in the second state (e.g., the abnormal state) (3611-Y). Operations 3607 and 3609 of the electronic device 130 may be performed as described above in FIG. 34, and thus, a repeated description thereof will be omitted.

According to various embodiments, the electronic device 130 (e.g., the processor 1409) may determine whether a condition associated with the inclination is satisfied (e.g., whether it corresponds to a specified angle range) in operation 3615, and may perform flash photographing in operation 3615-Y when the condition associated with the inclination is satisfied (3615—Y).

Hereinafter, examples of operations of the electronic device 130 according to various embodiments will be further described.

According to various embodiments, the electronic device 130 (e.g., the processor 1409) may perform photographing by moving an area, in which a focus set for the in vitro diagnostic device 110.

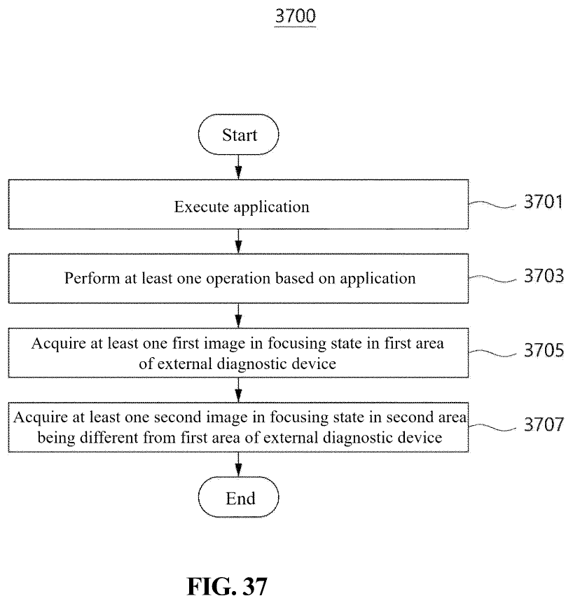

FIG. 37 is a flowchart 3700 illustrating an example of an operation of the electronic device 130 according to various embodiments. According to various embodiments, the operations illustrated in FIG. 37 are not limited to the order illustrated, and may be performed in various orders. Furthermore, according to various embodiments, at least one operation that is less or more than the operations illustrated in FIG. 37 may be performed.

According to various embodiments, the electronic device 130 (e.g., the processor 1409) may execute an application in operation 3701, and may perform at least one operation based on the application in operation 3703. For example, operations 3701 to 3703 of the electronic device 130 may be performed in the same manner as operations 3401 to 3403 described above, and thus a repeated description thereof will be omitted.

According to various embodiments, the electronic device 130 (e.g., the processor 1409) may acquire at least one first image while focusing on the first area of the in vitro diagnostic device 110 in operation 3705, and may acquire at least one second image while focusing on a second area that is different from the first area of the in vitro diagnostic device 110. For example, the electronic device 130 may acquire an image by using the camera 1401 while an area, in which the focus is set, is moved to the second area that is different from the first area, after acquiring an image using the camera 1401 while a focus is set on the first area of the in vitro diagnostic device 110. For example, the first area and the second area may be areas having different heights (i.e., stepped) from the lower surface of the in vitro diagnostic device 110. The meaning that the focus is set may mean that an image of a specific area (e.g., a first area or a second area of the in vitro diagnostic device 110) formed through a lens (not illustrated) in an interior of the camera 1401 is formed on an image sensor (not illustrated). When no focus is set, an image for a specific area is not formed on the image sensor (not illustrated), and thus, an image for the specific area may not be clearly acquired. In other words, an image of the specific area (e.g., the first area or the second area), to which the focus, is set may be captured more clearly than that of other areas, in which no focus is set. The position, in which the image of the specific area (e.g., the first area or the second area of the in vitro diagnostic device 110) is formed, may be formed based on a focal length of the lens of the camera 1401 of the electronic device 130 and a distance between the specific area (e.g., the first area or the second area) and the camera 1401 of the electronic device 130. Then, because the focal length of the lens of the camera 1401 of the electronic device 130 is uniquely pre-configured, the electronic device 130 may change a physical disposition of the specific area (e.g., the first area or the second area) and the lens (not illustrated) of the camera 1401 of the electronic device (e.g., adjust a distance between the specific areas due to adjustment of the position) to change a position, in which an image of the specific area (e.g., the first area or the second area) is formed. Accordingly, a focus may be set for each specific area (e.g., the first area, or the second area). Because the corresponding technology is a well-known technology, a more detailed description thereof will be omitted.

According to various embodiments, the electronic device 130 (e.g., the processor 1409) may set a focus for each area (e.g., the first area and the second area) of the in vitro diagnostic device 110 in different ways. For example, the electronic device 130 (e.g., the processor 1409) may select a method for setting a focus for each area depending on the height for each area (e.g., the first area and the second area) from the lower surface of the in vitro diagnostic device 110. The method for setting the focus may include an auto-focusing method and a manual focusing method. As an example, the electronic device 130 may set a focus for an area (e.g., the first area) having a relatively high height in an auto-focusing method and set a focus for an area (e.g., the second area) having a relatively low height in a manual focusing method. Then, the electronic device 130 may set a focus on the second area by moving the lens in the camera 1401 by a distance corresponding to a corresponding step based on a step between the pre-stored areas (e.g., the first area and the second area) as at least part of the operation of setting the focus in the manual focusing method.

Meanwhile, without being limited to the examples described above, the electronic device 130 may set a focus area in the same manner for each area. For example, the electronic device 130 may identify the position of the second area (e.g., the pad 220) from the image of the first area (e.g., a partial area of the housing 210) of the in vitro diagnostic device 110, which is captured in the auto-focusing state, and may perform an auto-focusing operation for the identified position of the second area (e.g., the pad 220).

According to various embodiments, the electronic device 130 (e.g., the processor 1409) may perform different functions for the images (e.g., the first image and second image) captured in different focus states. For example, the electronic device 130 (e.g., the processor 1409) may perform different functions for each image for each area according to the height of each area e.g., first area, and second area from the lower surface of the in vitro diagnostic device 110. As an example, the electronic device 130 may perform a function of determining whether the photographing condition (e.g., a photographing distance and a photographing inclination) of the electronic device 130 is satisfied based on the image for the first area, and may perform a function of acquiring an image of an area of the pad 220 for analyzing the diagnosis result of the in vitro diagnostic device 110 based on the image for an area (e.g., the second area) having a relatively low height.

Hereinafter, examples of operations of the electronic device 130 according to various embodiments will be further described.

According to various embodiments, the electronic device 130 (e.g., the processor 1409) may perform photographing by moving an area, in which a focus set for the in vitro diagnostic device 110.

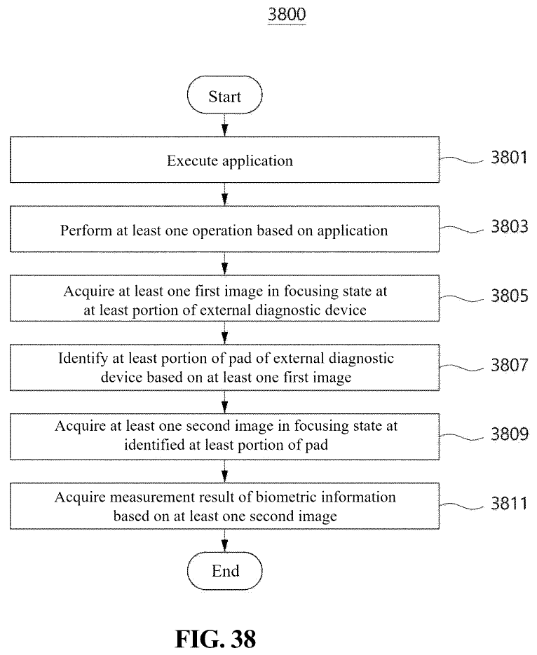

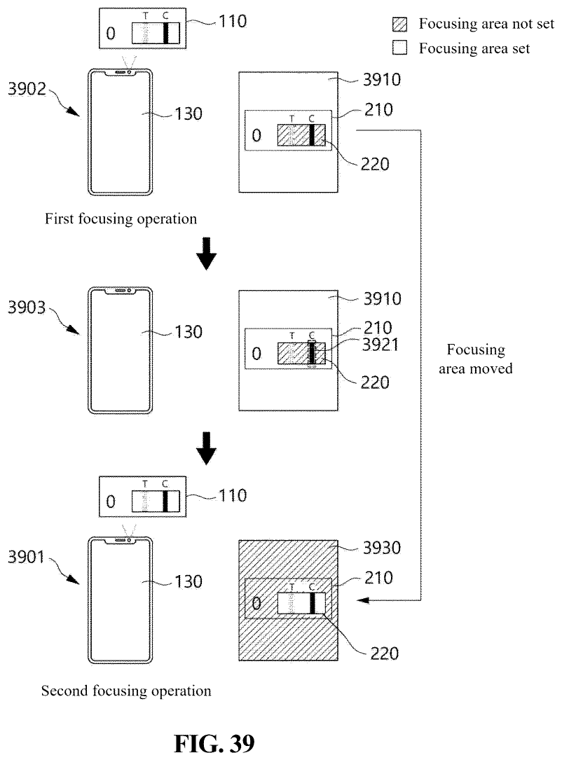

FIG. 38 is a flowchart 3800 illustrating an example of an operation of the electronic device 130 according to various embodiments. According to various embodiments, the operations illustrated in FIG. 38 are not limited to the order illustrated, and may be performed in various orders. Furthermore, according to various embodiments, at least one operation that is less or more than the operations illustrated in FIG. 38 may be performed. Hereinafter, FIG. 38 will be further described with reference to FIG. 39.

FIG. 39 is a view illustrating an example of an operation of performing photographing while moving an area, in which a focus of the electronic device 130 is set, according to various embodiments.

According to various embodiments, the electronic device 130 (e.g., the processor 1409) may execute an application in operation 3801, and may perform at least one operation based on the application in operation 3803. For example, operations 3801 to 3803 of the electronic device 130 may be performed in the same manner as operations 3401 to 3403 described above, and thus a repeated description thereof will be omitted.

According to various embodiments, the electronic device 130 (e.g., the processor 1409) may acquire at least one first image while focusing at least a portion of the housing of the in vitro diagnostic device 110 in operation 3805, may identify at least a portion of the pad 220 based on at least one first image in operation 3807, and may acquire at least one second image including the pad 220 while focusing on at least a portion (e.g., the control line 3921) of the identified pad 220. For example, while displaying the guide UI based on the application, the electronic device 130 may continuously acquire (or perform continuous photographing) a plurality of images while moving the area, in which the focus is set, by using the camera 1401 (e.g., the image sensor (not illustrated)). For example, as illustrated in 3901 of FIG. 39, the electronic device 130 may first perform an operation of photographing the first image 3910 while setting a focus on a portion (e.g., at least a portion of the housing 210) having the highest height (e.g., the first focusing operation of FIG. 39). The first focusing operation may be performed by the focusing operation (e.g., an auto-focusing operation) described in FIG. 37, and a repeated description thereof will be omitted. Referring to 3901 of FIG. 39, a focus area may be set on a portion (e.g., the housing 210) outside the pad 220 of an image of the first image 3910, based on a step between the pad 220 and the housing 210. As illustrated in 3902 of FIG. 39, the electronic device 130 may identify at least a partial area (e.g., the control line 3921) in the pad 220, based on the first image 3910. For example, the electronic device 130 may identify the position of at least a partial area in the in vitro diagnostic device 110 based on the configuration, physical structure, and/or the position of the visual marker identified from the first image 3910. For example, the electronic device 130 may identify the position of the control line 3921 of the pad 220. Because the color concentration of the test line expressed on the pad 220 may be low, the electronic device 130 may identify the position of the pad 210 more accurately by identifying the position of the control line 3921. However, without being limited to the described example, and another portion (e.g., a contour, a test line, and an area between the lines) may be identified. Accordingly, as illustrated in 3903 of FIG. 39, the electronic device 310 may acquire the second image 3930 by setting a focus area (e.g., a second focusing operation) on at least a partial area (e.g., the control line 3921) of the pad 220 and performing photographing while setting a focus on at least a partial area (e.g., the control line 3921) of the pad 220. Then, the second focusing operation may be performed by the focusing operation (e.g., a manual focusing operation) described in FIG. 37, and a repeated description thereof will be omitted. Referring to 3903 of FIG. 39, a focus area may be set at a portion of the image of the in vitro diagnostic device 110 of the second image 3930, so that a reaction result (e.g., the test line) in the pad 220 may be more clearly identified.

In addition, according to various embodiments, the electronic device 130 may identify a distance to pad 220 based on the step between the housing 210 and the pad 220, may sets a focus on at least the partial area (e.g., the control line 3921) of the pad 220 by moving the position of the lens of the camera 1401 so that the focus area corresponding to the distance to the identified pad 220 is set, and may acquire the second image 3930 by performing photographing.

According to various embodiments, the electronic device 130 (e.g., the processor 1409) may acquire a biometric information measurement value (e.g., a concentration value of the test line) based on at least one second image in operation 3811.

Hereinafter, examples of operations of the electronic device 130 according to various embodiments will be further described.

According to various embodiments, the electronic device 130 (e.g., the processor 1409) may perform photographing by moving an area, in which a focus set for the in vitro diagnostic device 110.

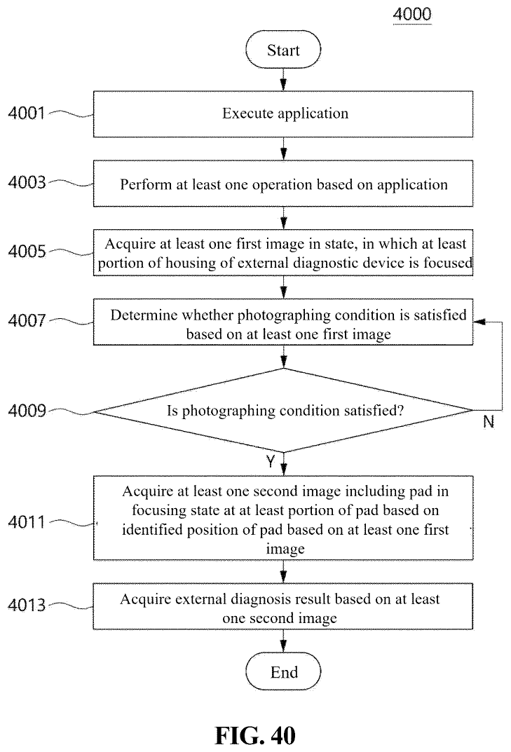

FIG. 40 is a flowchart 4000 illustrating an example of an operation of the electronic device 130 according to various embodiments. According to various embodiments, the operations illustrated in FIG. 40 are not limited to the order illustrated, and may be performed in various orders. Furthermore, according to various embodiments, at least one operation that is less or more than the operations illustrated in FIG. 40 may be performed.

According to various embodiments, the electronic device 130 (e.g., the processor 1409) may execute an application in operation 4001, and may perform at least one operation based on the application in operation 4003. For example, the electronic device 130 may perform an operation of providing at least one execution screen (e.g., a timer UI (e.g., the timer screen 1610 of FIG. 16), a guide UI (e.g., the guide screen 1620 of FIG. 16), and/or a result screen UI (e.g., the result screen 1630 of FIG. 16) based on execution of an application that is configured to provide the above-described in vitro diagnosis result, and/or providing at least one function based on the provided at least one execution screen.

According to various embodiments, the electronic device 130 (e.g., the processor 1409) may acquire at least one first image while focusing at least a portion of the housing of the in vitro diagnostic device 110 in operation 4005, and may determine whether a photographing condition (e.g., a photographing distance or a photographing inclination) is satisfied based on at least one first image in operation 4007. For example, the electronic device 130 may determine whether a photographing condition is satisfied by using a guide UI and/or an inclination sensor. The operation of determining whether the electronic device 130 satisfies the photographing condition may be performed as described above, and thus a repeated description thereof will be omitted.

According to various embodiments, when it is determined that the photographing condition is satisfied (4009—Y) in operation 4009, the electronic device 130 (e.g., processor 1409) may acquires at least one second image including the pad 220 while focusing on at least a portion of the pad 220, based on the position of the pad 220 identified based on the at least one first image in operation 4011, and may acquire a measurement result of a biometric information measurement value based on at least one second image in operation 4013. The electronic device 130 may start photographing (e.g., control the state of an automatic photographing or a graphic object for photographing as an active state) by using the guide UI and/or a tilt sensor. Then, as described above, the electronic device 130 may perform a photographing operation in a state, in which a focus is set on the pad 220 (or the membrane).

Hereinafter, examples of operations of the electronic device 130 according to various embodiments will be further described.

According to various embodiments, the electronic device 130 (e.g., the processor 1409) may provide a guide UI for photographing a plurality of pads, and may provide information on the in vitro diagnosis result based on a comparison of information analyzed for the plurality of pads.

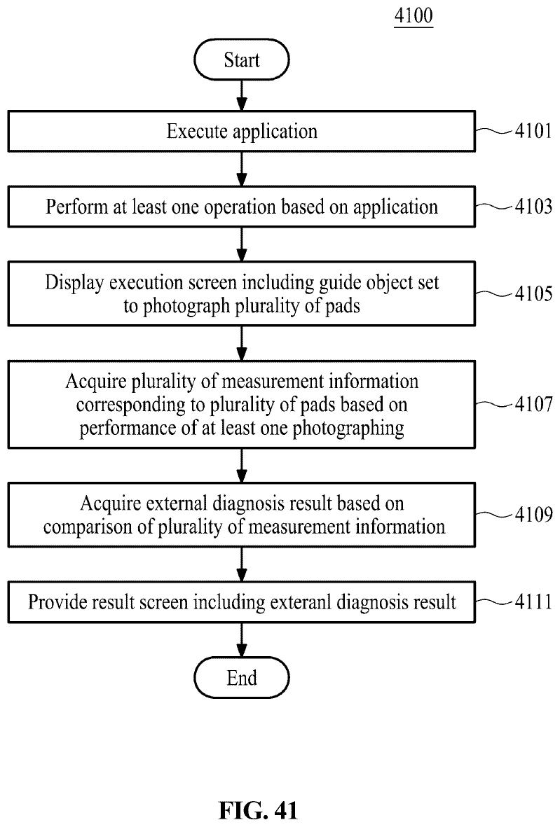

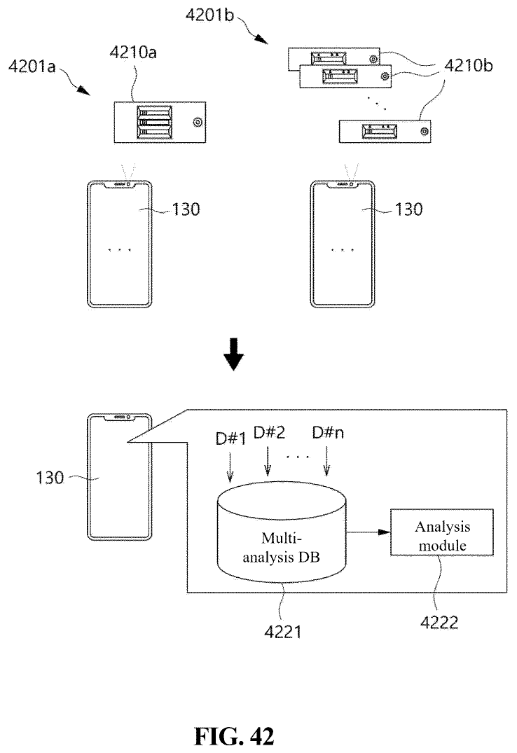

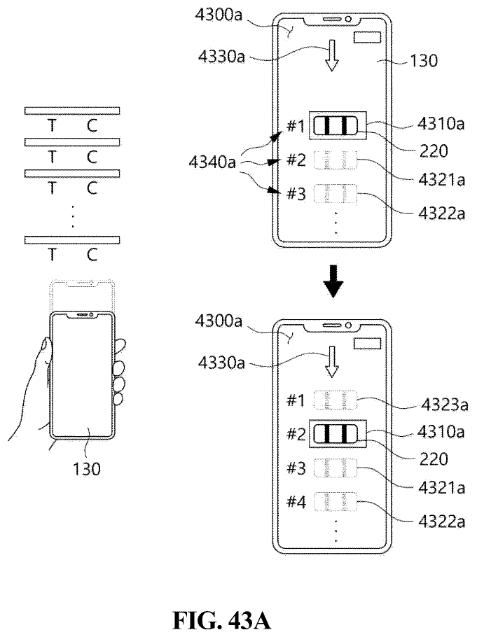

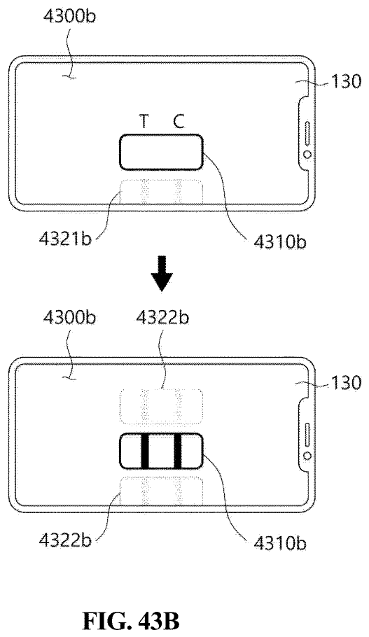

FIG. 41 is a flowchart 4100 illustrating an example of an operation of the electronic device 130 according to various embodiments. According to various embodiments, the operations illustrated in FIG. 41 are not limited to the order illustrated, and may be performed in various orders. Furthermore, according to various embodiments, at least one operation that is less or more than the operations illustrated in FIG. 41 may be performed. Hereinafter, FIG. 41 will be further described with reference to FIGS. 42 and 43.

FIG. 42 is a view illustrating an example of an operation of photographing a plurality of pads of the electronic device 130 according to various embodiments and an operation of providing information on an in vitro diagnosis result based on information analyzed for the plurality of pads. FIG. 43A is a view illustrating an example of a guide UI based on a portrait mode provided by the electronic device 130 according to various embodiments. FIG. 43B is a view illustrating an example of a guide UI based on a landscape mode provided by the electronic device 130 according to various embodiments.

According to various embodiments, the electronic device 130 (e.g., the processor 1409) may execute an application in operation 4101, and may perform at least one operation based on the application in operation 4103. For example, operations 4101 to 4103 of the electronic device 130 may be performed in the same manner as operations 3401 to 3403 described above, and thus a repeated description thereof will be omitted.

processor 1409) may displays an execution screen including a guide object that is set to photograph the plurality of pads 220 in operation 4105, and may acquire a plurality of measurement information corresponding to the plurality of pads 220 based on performance of at least one photographing in operation 4107. For example, photographing a plurality of pads (or membranes) by using the electronic device 130 may include photographing the in vitro diagnostic device 4210a including a plurality of pads, as shown in 4201a of FIG. 42, and/or photographing a plurality of in vitro diagnostic devices 4210b including at least one pad, as illustrated in 4201b of FIG. 42. Then, as illustrated in FIGS. 43A and 43B, the electronic device 130 may provide a plurality of photographing guide UIs 4300a and 4300b (e.g., the first guide object 1621a of FIG. 16) for photographing the plurality of pads, and may guide photographing of the plurality of pads based on the provided photographing guide UIs 4300a and 4300b. For example, the plurality of photographing guide UIs 4300a and 4300b may be configured to provide a function for guiding to sequentially photograph the plurality of pads. Then, the plurality of photographing guide UIs may be provided based on a screen orientation of the electronic device 130, and FIG. 43a illustrates an example 4300a of a plurality of photographing guide UIs corresponding to a portrait mode, and FIG. 43b illustrates an example 4300b of a plurality of photographing guide UIs corresponding to a landscape mode. Hereinafter, examples of the plurality of photographing guide UIs 4300a and 4300b will be described.

According to various embodiments, the plurality of photographing guide UIs 4300a and 4300b may include the first graphic objects 4310a and 4310b for guiding the pad 220 that is to be photographed at the current time point, at least one second graphic object 4321a, 4322a, 4323a, 4324a, 4321b, 4322b, and 4323b for providing information on the other remaining pads 220, at least one text 4340a, and the other graphic objects 4330a. Meanwhile, without being limited to the described and/or illustrated examples, the plurality of photographing guide UIs 4300a and 4300b may be configured to include more graphic objects and/or texts, or may include fewer graphic objects and/or texts.

For example, the first graphic objects 4310a and 4310b may be a first guide object 1621a included in the above-described guide UI e.g. (the guide screen 1620 of FIG. 16). Then, when a specific pad (e.g., the pad 220 located at the uppermost end, and/or the pad 220 of the specific in vitro diagnostic device 110) is photographed based on the first graphic objects 4310a and 4310b, the first graphic objects 4310a and 4310b may be displayed in positions corresponding to the pad 220 that is to be photographed in the next order in the plurality of photographing guide UIs 4300a and 4300b. Accordingly, the user may identify the specific pad 220 that is to be photographed in the next order based on the first graphic objects 4310a and 4310b, of which the positions have been moved. When photographing conditions (e.g., photographing distance, and photographing inclination) are satisfied based on the first graphic objects 4310a and 4310b, the electronic device 130 may automatically perform photographing or control a state of a graphic object for photographing to an active state so that photographing may be performed.

For example, the at least one second graphic object 4321a, 4322a, 4323a, 4324a, 4321b, 4322b, and 4323b may be a graphic object that is configured to provide information (e.g., a position or an order) on other pads that have not yet been photographed. For example, as illustrated in FIGS. 43A and 43B, the at least one second graphic object 4321a, 4323a, 4324a, 4321b, and 4323b may be configured as an image for the pad. For example, referring to FIGS. 43A and 43B, the electronic device 130 may display a plurality of photographing guide UIs 4300a and 4300b, in which at least one translucent image 4321a, 4322a, and 4321b corresponding to a pad that is to be sequentially photographed in a next order is sequentially disposed, below the first graphic objects 4310a and 4310b. Meanwhile, without being limited to the illustrated and/or illustrated examples, the pad image may be displayed in a color not in a translucent state. Furthermore, as an example, when a specific pad is photographed, the electronic device 130 may display the first graphic objects 4310a and 4310b instead of the images 4321a and 4321b for the pad that is to be photographed in a next order. However, without being limited to the illustrated and/or the described examples, and the electronic device 130 may visually highlight (e.g., emphasize a contour, and change a color) the images 4321a and 4321b while displaying the images 4321a and 4321b for the pad.

According to various embodiments, at least one text may include a text 4340a that indicates identification information on the pad and/or a text (e.g., “put the electronic device down sequentially”) for a photographing guide, and may further include various types of texts or include fewer texts without being limited to the examples described.

According to various embodiments, in addition to the above-described visual contents, graphic objects for a photographing guide, such as an arrow 4330a, may be further provided to the plurality of photographing guide UIs 4300a and 4300b.

According to various embodiments, when the in vitro diagnostic device 110 including a plurality of pads is photographed (e.g., 4201a of FIG. 42), the electronic device 130 (e.g., the processor 1409) may provide the plurality of photographing guide UIs 4300a and 4300b based on the acquisition of information on the in vitro diagnostic device 110. For example, the electronic device 130 (e.g., the processor 1409) may determine the type of the in vitro diagnostic device 110 based on the product information acquired based on the QR code, and may provide the plurality of photographing guide UIs 4300a and 4300b described above when the type of the in vitro diagnostic device 110 is of a type including a plurality of pads. As another example, the electronic device 130 (e.g., the processor 1409) may acquire an image for the pad from an image of the in vitro diagnostic device 110 pre-stored in the memory 1411 based on the product information acquired based on the QR code, and may acquire at least one graphic object 4321a, 4322a, 4323a, 4321b, 4321b, and 4323b described above based on the acquired image and provide it onto the plurality of photographing guide UIs 4300a and 4300b.

According to various embodiments, when the plurality of in vitro diagnostic devices 4210b are photographed (e.g., 4201b of FIG. 42), the electronic device 130 (e.g., the processor 1409) may provide the plurality of photographing guide UIs 4300a and 4300b based on an input of the user. or example, the electronic device 130 may provide graphic objects (e.g., icons) that causes display of the plurality of photographing guide UIs 4300a and 4300b on the execution screen, and when the graphic object is selected, the plurality of photographing guide UIs 4300a and 4300b may be provided. For example, the electronic device 130 may provide graphic objects for switching to the plurality of photographing guide UIs onto the guide UI. Then, the electronic device 130 may acquire information on the number of times of photographing of the pad 220 from the user, and may provide the number of graphic objects (e.g., the first graphic object, and the second graphic object) corresponding to the acquired number of times. Alternatively, without being limited to the example described, the electronic device 130 may be configured to provide a graphic object for ending photographing while providing infinite graphic objects, and to end the photographing when the graphic object for ending the photographing is selected.

According to various embodiments, the electronic device 130 (e.g., the processor 1409) may acquire a diagnosis result based on comparison of a plurality of pieces of measurement information in operation 4109, and may provide a result screen including the diagnosis result in operation 4111. The electronic device 130 may classify a plurality of images acquired based on performance of a plurality of photographing while providing a plurality of photographing guide UIs 4300a and 4300b into the same group, and may perform an operation of analyzing an in vitro diagnosis result based on the plurality of images. For example, as illustrated in FIG. 4202 of FIG. 42, the electronic device 130 may sequentially store a plurality of images D #1, D #2, . . . , and D #n that are sequentially photographed in a DB (e.g., a multi-analysis DB 4221) that is allocated to the same group. The electronic device 130 (e.g., an analysis module 4222) may acquire the plurality of images D #1, D #2, . . . , and D #n from the DB 4221 after ending photographing, and may acquire a plurality of biometric information measurement values by analyzing the plurality of acquired images. The electronic device 130 may acquire information on the in vitro diagnosis result based on a comparison of the acquired biometric information measurement values.

Hereinafter, examples of operations of the electronic device 130 according to various embodiments will be further described.

According to various embodiments, an example of an operation of the electronic device 130 (e.g., the processor 1409) of acquiring an in vitro diagnosis result based on a plurality of images corresponding to a plurality of pads will be described.

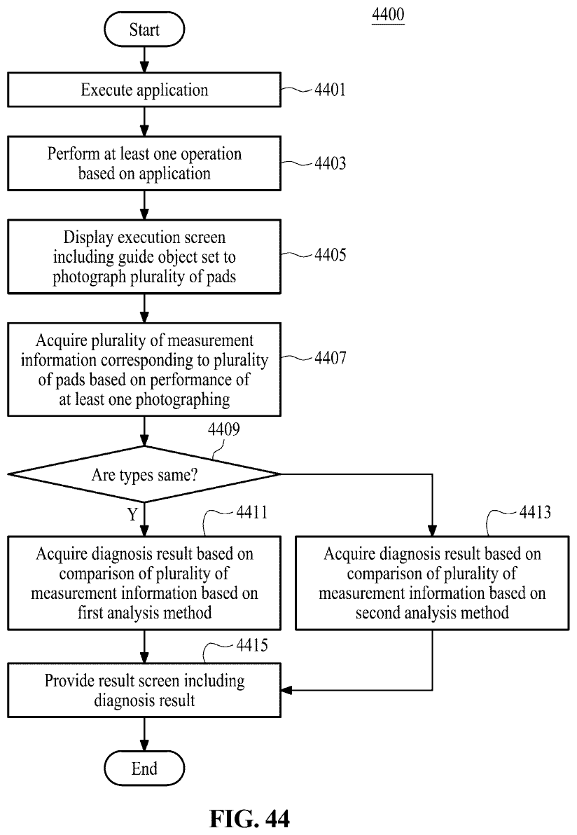

FIG. 44 is a flowchart 4400 illustrating an example of an operation of the electronic device 130 according to various embodiments. According to various embodiments, the operations illustrated in FIG. 44 are not limited to the order illustrated, and may be performed in various orders. Furthermore, according to various embodiments, at least one operation that is less or more than the operations illustrated in FIG. 44 may be performed.

According to various embodiments, the electronic device 130 (e.g., the processor 1409) may execute an application in operation 4401, and may perform at least one operation based on the application in operation 4403. For example, the electronic device 130 may perform an operation of providing at least one execution screen (e.g., a timer UI (e.g., the timer screen 1610 of FIG. 16), a guide UI (e.g., the guide screen 1620 of FIG. 16), and/or a result screen UI (e.g., the result screen 1630 of FIG. 16) based on execution of an application that is configured to provide the above-described in vitro diagnosis result, and/or providing at least one function based on the provided at least one execution screen.

According to various embodiments, the electronic device 130 (e.g., the processor 1409) may display an execution screen (e.g., a plurality of photographing guide UIs 4300a and 4300b) including a guide object that is set to photograph a plurality of membranes (or a plurality of pads), and may acquire a plurality of measurement information corresponding to a plurality of membranes (or a plurality of pads) based on performance of at least one photographing.

According to various embodiments, the electronic device 130 (e.g., the processor 1409) may determine whether the types of measured biometric information measurement values are the same in operation 4409, may acquire a diagnosis result based on comparison of a plurality of measurement information based on a first analysis method in operation 4411 when the types are the same (4409—Y), and may acquire a diagnosis result based on a comparison of a plurality of measurement information based on the second analysis method in operation 4413.

For example, in the case of the same type of biometric information measurement value, the electronic device 130 (e.g., the processor 1409) may calculate an average value for the plurality of biometric information measurement values and acquire an in vitro diagnosis result based on the calculated average value.

As another example, in the case of different types of biometric information measurement values, the electronic device 130 (e.g., the processor 1409) may acquire pre-configured information (e.g., an artificial intelligence model, and/or a look-up table) to acquire in vitro diagnosis results based on each of the biometric information measurement values, and may acquire in vitro diagnosis results based on each of the acquired information and a plurality of measurement values of biometric information. Then, when the difference between different measurement values of biometric information is a factor that affects the in vitro diagnosis results, the electronic device 130 may have information for correcting the in vitro diagnosis results corresponding to the difference between the above measurement values of biometric information. Accordingly, in the case of different types of measurement values of biometrics information, the electronic device 130 may further determine whether the types of the plurality of measurement values of biometric information require correction, may acquire correction information corresponding to the difference value of the plurality of measurement values of biometric information, and may use it to acquire the in vitro diagnosis result.

As another example, in the case of different types of measurement values of biometric information, the electronic device 130 (e.g., the processor 1409) may use each of the measurement values of biometric information as a purpose for verifying the in vitro diagnosis results (e.g., verification of positivity). For example, when the first biometric information measurement value indicates the amount of antibodies and the second biometric information measurement value indicates the amount of neutralized lutein, the electronic device 130 may determine the in vitro diagnosis result (e.g., positive or negative) based on a first biometric information measurement value, but when the in vitro diagnosis result is positive, the positivity may be verified based on the second biometric information measurement value (e.g., determined as positive when the amount of neutralized lutein is below a threshold value, or determined as positive when the amount of neutralized lutein is above the threshold value.

According to various embodiments, the electronic device 130 (e.g., the processor 1409) may provide a result screen including a diagnosis result in operation 4415.

Hereinafter, examples of operations of the electronic device 130 according to various embodiments will be further described.

According to various embodiments, the electronic device 130 may perform functions in different modes depending on whether the electronic device 130 is gripped (or held). The mode of the electronic device 130 may include a user grip mode and a holder holding mode.

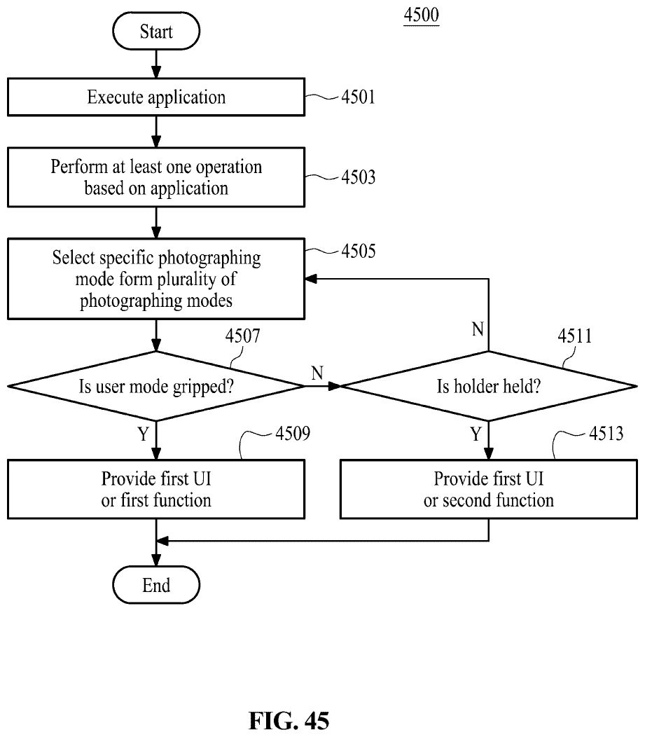

FIG. 45 is a flowchart 4500 illustrating an example of an operation of the electronic device 130 according to various embodiments. According to various embodiments, the operations illustrated in FIG. 45 are not limited to the order illustrated, and may be performed in various orders. Furthermore, according to various embodiments, at least one operation that is less or more than the operations illustrated in FIG. 45 may be performed.

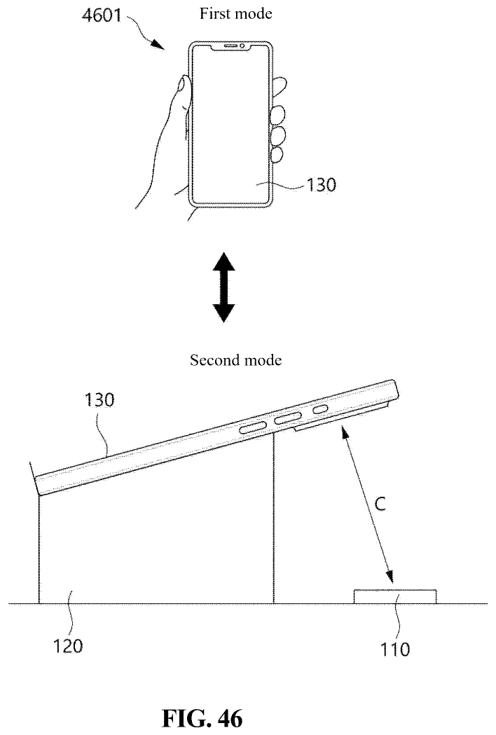

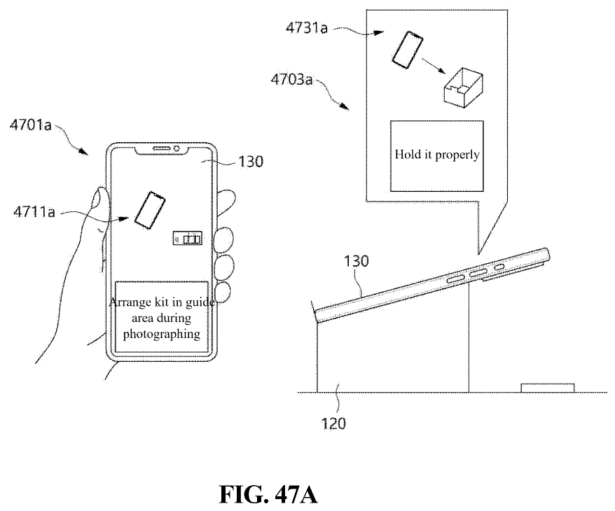

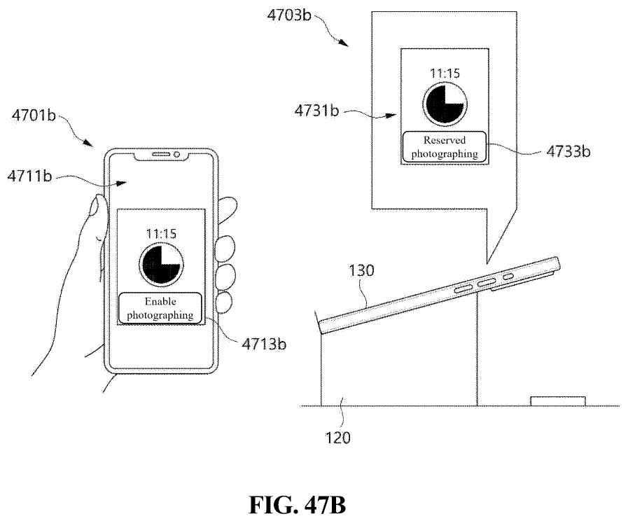

FIG. 46 is a view illustrating an example of an operation of performing a function based on different modes according to whether the electronic device 130 is gripped (or held) according to various embodiments. FIG. 47A is a view for explaining an example of a holder holding mode of the electronic device 130 according to various embodiments. FIG. 47B is a view for explaining an example of a user grip mode of the electronic device 130 according to various embodiments.

According to various embodiments, the electronic device 130 (e.g., the processor 1409) may execute an application in operation 4501, and may perform at least one operation based on the application in operation 4503. For example, operations 4501 to 4503 of the electronic device 130 may be performed in the same manner as operations 3401 to 3403 described above, and thus a repeated description thereof will be omitted.

According to various embodiments, the electronic device 130 (e.g., the processor 1409) may identify a specific photographing mode from a plurality of photographing modes in operation 4505. The plurality of photographing modes may include a user grip mode for photographing while the user grips the electronic device 130, and a holder holding mode for photographing while the electronic device 130 is held in the holder 120. In an embodiment, the electronic device 130 may identify whether it is in a holder holding mode or a user grip mode by using a sensor as at least a portion of an operation of identifying a specific photographing mode, among a plurality of photographing modules. For example, the electronic device 130 may determine whether it is a holder holding mode or a user grip mode based on a pattern of values sensed by using a sensor (e.g., an inclination sensor or an angular velocity sensor), and/or patterns of the values. For example, a pattern, in which a value that is sensed by using a sensor is a specific value and/or in a specific range, may be formed in the holder holding mode, and a value that is sensed by using a sensor may be formed in a variable pattern in the user grip mode. The electronic device 130 may store information on a corresponding pattern in advance, and may identify a state (e.g., holder holding, user grip) corresponding to the pattern of the identified value based on a comparison of the pre-stored information with a pattern of a value identified by using a sensor. In another embodiment, the electronic device 130 may identify a specific photographing mode, among a plurality of photographing modes, based on an input for the user to select a specific photographing mode (e.g., a user grip mode, or a holder holding mode) based on the application.

According to various embodiments, the electronic device 130 (e.g., the processor 1409) may perform an operation of determining whether it is to be held or held before providing the guide UI.

According to various embodiments, the electronic device 130 (e.g., the processor 1409) may provide at least one of the first UI and the first function in operation 4509 when the photographing mode is determined to be the user grip mode (4507—Y), in operation 4507. According to various embodiments, the electronic device 130 (e.g., the processor 1409) may provide at least one of the second UI and the second function in operation 4513 when the photographing mode is determined to be the holder holding mode in operation (4511—Y) in operation 4511. For example, the electronic device 130 may set the mode of the electronic device 130 to the user grip mode when the user grip mode is identified, may set the mode of the electronic device 130 to the holder holding mode when the holder holding mode is identified, and may perform an operation of providing a UI and/or function corresponding to the set mode. For example, as illustrated in Table 1 below, the electronic device 130 may perform an operation of providing a UI and/or function corresponding to the set mode.

TABLE 1

| Mode | Holder | Middle-classification | Explanation |
|---|---|---|---|
| Holder | UI | Manual UI | Provision of manual |
| holding | | | for photographing |
| mode | | | holder |
| | | Timer UI | Same |
| | | Guide UI | Including only guide |
| | | | zone |
| | | Result screen UI | Same |
| | Function | Reservation | Function for automatic |
| | | photographing | photographing based |
| | | function | on ending of timer |
| | | | Preset to enabling |
| | | | (changeable) |
| | | Focus setting | Set to preset focal |
| | | function | distance in manual |
| | | | focusing mode |
| User grip | UI | Manual UI | Provision of manual for |
| mode | | | manual photographing |
| | | Timer UI | Same |
| | | Guide UI | Guide zone + |
| | | | inclination guide |
| | | Result screen UI | Same |
| | Function | Reservation | Function for automatic |
| | | photographing | photographing based |
| | | function | on ending of timer |
| | | | Preset to disenabling |
| | | | (changeable) |
| | | Focus setting | Auto-focusing mode |
| | | function | |

For example, referring to FIG. 47A, the electronic device 130 (e.g., the processor 1409) may display an execution screen including a manual (e.g., including the phrase “Please adjust the kit to the guide area when photographing”) for guiding photographing of the user as illustrated in 4701a of FIG. 47A when the photographing mode of the electronic device 130 is a user holding mode, and may display an execution screen including a manual (e.g., including the phrase “Please hold correctly”) to guide the holding of the electronic device 130 on the holder 120 when the photographing mode is the holder holding mode as illustrated in 4703 of FIG. 47A.

Meanwhile, the UIs and functions described in Table 1, and FIGS. 47A and 47B are only examples, and may be configured with various UIs and functions without being limited to the description and/or illustration.

Hereinafter, examples of operations of the electronic device 130 according to various embodiments will be further described.

According to various embodiments, the electronic device 130 may provide a UI based on a time point, at which the photographing mode of the electronic device 130 is identified.

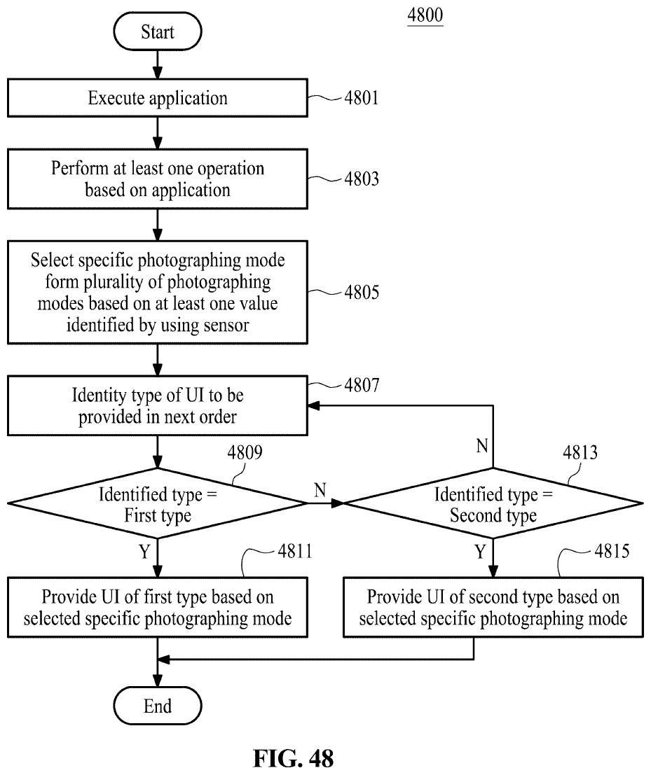

FIG. 48 is a flowchart 4800 illustrating an example of an operation of the electronic device 130 according to various embodiments. According to various embodiments, the operations illustrated in FIG. 45 are not limited to the order illustrated, and may be performed in various orders. Furthermore, according to various embodiments, at least one operation that is less or more than the operations illustrated in FIG. 48 may be performed.

According to various embodiments, the electronic device 130 (e.g., the processor 1409) may execute an application in operation 4801, and may perform at least one operation based on the application in operation 4803.

According to various embodiments, the electronic device 130 (e.g., the processor 1409) may identify a specific photographing mode from a plurality of photographing modes in operation 4805, and may identify the type of the UI that is to be provided in operation 4807. The type of UI may include (e.g., a timer UI (e.g., a timer screen 1610 of FIG. 16), a guide UI (e.g., a guide screen 1620 of FIG. 16), and/or a result screen UI (e.g., a result screen 1630 of FIG. 16). The electronic device 130 may perform an operation of identifying the type of a UI that is to be provided at a time point, at which a specific photographing mode is selected, and/or an operation of identifying the type of a UI that is to be provided in the next order of the UI that is being provided at a time point, at which the specific shooting mode is selected, as at least part of an operation of identifying the type of a UI that is to be provided, The electronic device 130 may store information on the order of provision of UIs for the photographing modes (e.g., a user grip mode and a holder holding mode) in the memory 1411 in advance, and may identify the type of a UI that is to be provided at a time point, at which a specific photographing mode is selected, or the type of an UI that is to be provided in a next order.

According to various embodiments, when the identified type is the first type (4809—Y) in operation 4809, the electronic device 130 (e.g., the processor 1409) may provide a first type UI based on the selected specific photographing mode in operation 4811, and when the identified type is the second type (48013—Y), a second type UI may be provided based on the selected specific photographing mode in operation 4815. In an embodiment, when it is identified that the photographing mode has been changed, the electronic device 130 may change the currently displayed UI to the same type UI corresponding to the changed photographing mode to display it. In another embodiment, the electronic device 130 may provide a specific UI depending on the photographing mode that is determined before the specific UI is displayed. As an example, the electronic device 130 may provide a manual UI corresponding to a mode corresponding to a user grip state when a user grip has been identified before the manual UI is provided, and may perform an operation of providing a timer UI corresponding to a mode corresponding to the holder holding state when it is identified as holder holding again before the timer UI is provided.

According to various embodiment, a packaging container, in which an inner space is configured to accommodate an in vitro diagnostic device, may include a body defining the inner space, and including an upper surface, a lower surface, and a plurality of side surfaces connecting the upper surface and the lower surface, and a first structure that may be cut at an angle in a first range from the lower surface of the body is formed on each of a first side surface and a second side surface being opposite to the first surface, among the plurality of side surfaces, the angle in the first range is greater than or equal to 5 degrees and less than 30 degrees, a part, in which the electronic device of the user may be held at an angle in the first range is formed on the remaining part of the body when a portion of the body or the entire body is cut by the above structure, and at least one support part that may support the electronic device is formed on at least one of the plurality of side surfaces.

According to various embodiment, in the packaging container, a first structure may be formed in areas of the first side surface and the second side surface, which correspond to a range of not less than 0.6 and less than 0.8 of a height from the lower surface of the body to the upper surface of the body.

According to various embodiment, in the packaging container, the first structure may define an angle of about 15 degrees with the lower surface of the body.

According to various embodiment, in the packaging container, a second structure that may be cut may be formed on the third side surface, a third structure that may be cut may be formed on the fourth side surface, and the second structure and the first structure may be connected to each other.

According to various embodiment, in the packaging container, a height of the third side surface may be smaller than a height of the fourth side surface, a partial area of the second structure formed on the third side surface may be configured in a form protruding upward, and when a portion of the body or the entire body is cut, the at least one support part includes at least one first support part formed on the third side surface by the partial area of the second structure.

According to various embodiment, in the packaging container, at least a partial area of the fourth side surface may include a material having a high absorption performance and a high transmission performance of light compared to at least one of the other side surfaces.

According to various embodiment, in the packaging container, the at least partial area of the fourth side surface may be spaced apart from the lower surface of the body by a specific distance.

According to various embodiment, in the packaging container, at least partial area of the fourth side surface is configured to be cut, and at least partial area of the fourth side surface is configured to be used to guide disposition of an in vitro diagnostic device.

According to various embodiment, in the packaging container, a partial area of the first structure formed on the first side surface and the second side surface may be configured in a form protruding upward, and when a portion of the body or the entire body is cut, the at least one support part includes at least one second support part formed on the first side surface and the second side surface by the partial area of the first structure.

According to various embodiment, in the packaging container, a folding structure for folding into the inner space of the body may be formed in an area, in which the partial area of the first structure is connected to the remaining portions of the body.

According to various embodiment, in the packaging container, a partial area of a structure formed two or more side surfaces connected to each other, among the plurality of side surfaces, may be configured in a form that protrudes upward, and when a portion of the body or the entire body is cut, the at least one support part includes at least one second support part formed on the two or more side surfaces.

According to various embodiment, in the packaging container, a height of the third side surface may be smaller than a height of the fourth side surface, a foldable fourth structure may be formed on the third side surface, and a fifth structure that may be cut may be formed on the fourth side surface.

According to various embodiment, in the packaging container, the portion of the body may be rotated in one direction by the fourth structure such that an inner surface of the portion of the body is exposed to an outside, and when the electronic device is hold at the remaining portions while the portion of the body is rotated in the one direction, at least a portion of the inner surface of the portion of the body may support at least portion of the electronic device.

According to various embodiment, in the packaging container, at least a partial area of a surface connected to the fourth structure of the portion of the body may be configured to be cut, and while the portion of the body is rotated in the one direction, the at least partial area of the surface connected to the fourth structure may be configured to contact the remaining portions of the body.

According to various embodiment, in the packaging container, a material of a high frictional coefficient may be disposed on a bottom surface of the body.

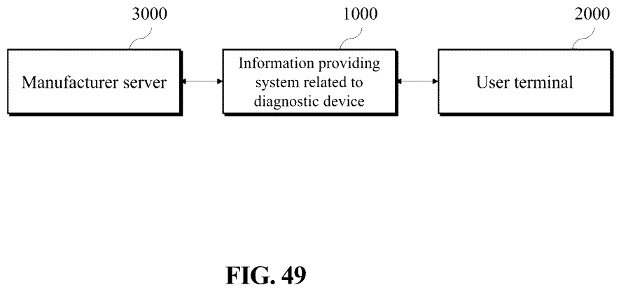

FIG. 49 is an environmental view of an information providing system related to a diagnostic device according to an embodiment.

Referring to FIG. 49, an information providing system 1000 (hereinafter, referred to as a system) related to a diagnostic device may communicate with a user terminal 2000 and the manufacturer server 3000 to transmit and receive data. FIG. 49 illustrates that the number of user terminals 2000 that communicates with the system 1000 and the number of manufacturer servers 3000 are one, but is not limited thereto, the system 1000 may communicate with two or more user terminals and two or more manufacturers.

The system 1000 may include a server that provides a platform service related to the diagnostic device. Specifically, the system 1000 may include a server that provides a digital vaccine platform service. Then, the digital vaccine platform may mean a platform that transmits, analyzes, and processes data related to the diagnostic device through communication and provides services to users and manufacturers by using data related to the diagnostic device. For example, when a user uploads an image acquired by photographing the diagnostic device to the platform, the platform may analyze the image and provide analysis contents to the user, and provide contents on a usage time point, Lot information, an error, a defect, and a usage of the diagnostic device to the manufacturer.

The system 1000 may acquire the user information and the specification information of the diagnostic device from the user terminal 2000. Then, the user information may include at least one of a gender, an age, an area, a weight, a height, a body mass coefficient, an exercise amount, a sleep amount, information on possessed diseases, information on a medication, an interested diagnostic item, attending hospital information, and a health diagnosis result. Furthermore, the specification information of the diagnostic device may include at least one of a name, a manufacturer, and a possible diagnostic item of the diagnostic device. The acquisition of the specification information of the diagnostic device from the user terminal 2000 may be performed by operation 1510 of FIG. 15, operation 1710 of FIG. 17, or operation 1910 of FIG. 19.

The system 1000 may transmit use manual data of the diagnostic device to the user terminal 2000 based on the specification information of the diagnostic device. The use manual data of the diagnostic device may include a usage of the diagnostic device, standby time information after introduction of the specimen, and the like.

The system 1000 may acquire manufacturing characteristic data from the user terminal 2000 or the manufacturer server 3000. The manufacturing characteristic data is characteristic data for each lot measured in a final quality inspection when the diagnostic device is produced in a manufacturer, and may include a calibration value, a calibration factor, and the like. Specifically, the system 1000 may acquire manufacturing characteristic data corresponding to the diagnostic device used by the user from the manufacturer server 3000 based on the specification information of the diagnostic device acquired from the user terminal 2000.

The system 1000 may acquire result data of the diagnostic device result data from the user terminal 2000. The result data of the diagnostic device may be an image or image data that is acquired by photographing the diagnostic device, into which the specific collected from the user's body is introduced.

Acquisition of the result data of the diagnostic device from the user terminal 2000 may be performed in operation 3210 of FIG. 32. Furthermore, the result data of the diagnostic device may be the first image or the second image of FIG. 32, but is not limited thereto.

The system 1000 may correct the result data of the diagnostic device by using the manufacturing characteristic data. Specifically, the system 1000 may correct an image acquired by photographing the diagnostic device by using a calibration value and a calibration factor included in the manufacturing characteristic data.

The system 1000 may analyze the result data of the diagnostic device. The system 1000 may generate healthcare solution data based on an analysis result of the result data of the diagnostic device. Specifically, the system 1000 may generate healthcare solution data by inputting the result data of the diagnostic device and user information into an artificial intelligence model. Then, the healthcare solution data may include an exhaustion therapy, a diet therapy, a nutritional supplement or a health functional food intake guide, a digital therapeutic agent, additional diagnostic test item information, and self-diagnosis cycle information using the diagnostic device. The system 1000 may transmit the generated healthcare solution data to the user terminal 2000.

For example, when the analysis result based on the result data of the diagnostic device is 70% of the value for item “A”, the system 1000 has 70% of the value for item A and may generate user-customized healthcare solution data for users with rhinitis disease, which is a pre-existing disease. Then, the user-customized healthcare solution data may include recipes and diets using ingredients for lowering the value of item “A”, exercise plans three times a week, and self-diagnosis plans using diagnostic devices twice a month.

The user terminal 2000 may be a device of a user using a platform provided by the system 1000. Specifically, the user terminal 2000 may be a terminal of the user or a server of a computer of the user. The user may use a service that is provided by the system 1000 through the user terminal 2000. Specifically, the user may photograph the diagnostic device through the user terminal 2000, and may manage his or her health through a health management solution provided by the system 1000.

The system 1000 may communicate with the manufacturer server 3000 to transmit and receive data related to the usage of the diagnostic device. Specifically, the system 1000 may generate error or defect information of the diagnostic device based on the result data of the diagnostic device acquired from the user terminal 2000. Furthermore, the system 1000 may generate diagnostic device usage prediction data by using self-diagnosis cycle information included in healthcare solution data transmitted to the user terminal 2000. The system 1000 may transmit the generated error/defect information and diagnostic device usage prediction data to the manufacturer server 3000.

The manufacturer server 3000 may be a device of a manufacturer using a platform provided by the system 1000. Specifically, the manufacturer server 3000 may be a server of a company that manufactures a diagnostic device. Then, the manufacturer is a company that manufactures a diagnostic device that may be handled on a platform, and may include not only domestic companies but also foreign companies. The manufacturer may acquire information on the usage and defect of the diagnostic device acquired through the manufacturer server 3000, and may identify the manufacturing equipment of the diagnostic device or correct the production amount of the diagnostic device by using the information.

In addition to the above example, the system 1000 may provide statistical analysis data using result data of the diagnostic device, a market usage, defect trends, customer complaints, customer need information, diagnostic device purchase place information, online order information, and the like to the manufacturer server 3000.

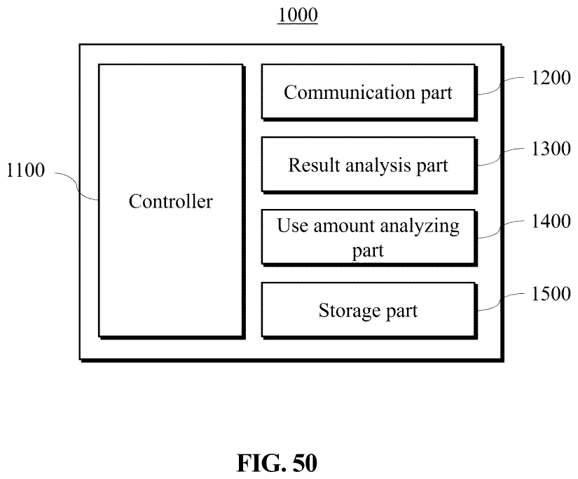

FIG. 50 is a block diagram of a system for providing information related to a diagnostic device according to an embodiment.

Referring to FIG. 50, an information providing system 1000 related to a diagnostic device according to an embodiment may include a controller 1100, a communication part 1200, a result analyzing part 1300, a usage analyzing part 1400, and a storage part 1500.

FIG. 50 illustrates five components included in the system 1000, but the illustrated components are not essential, and the system 1000 may have more or fewer components. Furthermore, the components of the system 1000 may be physically operated by one processor or may be operated by processors that are distributed for the functions.

The controller 1100 may manage overall operations of the system 1000. The controller 1100 may be a control processor that outputs a control command. Specifically, the controller 1100 may execute the operations of departments by sending a control command to the communication part 1200, the result analyzing part 1300, the usage analyzing part 1400, and the storage part 1500.

When otherwise particularly mentioned below, it may be interpreted that the operation of the system 1000 is performed under the control of the controller 1100.

The communication part 1200 may connect and communicate the system 1000 and an external device. For example, the communication part 1200 may exchange data with the user terminal 2000 and the manufacturer server 3000.

According to an embodiment, the communication part 1200 may receive user information, specification information of the diagnostic device, and result data of the diagnostic device from the user terminal 2000. Furthermore, the communication part 1200 may transmit use manual data of the diagnostic device and healthcare solution data to the user terminal 2000. Furthermore, the communication part 1200 may transmit usage prediction data of the diagnostic device to the manufacturer server 3000.

The communication part 1200 may be a communication module that supports at least one of a wired communication method and a wireless communication method. For example, the communication part 1200 may acquire data from an external device through a communication method, such as Bluetooth, Zigbee, Bluetooth low energy (BLE), RFID, and the like, but is not limited thereto.

The result analyzing part 1300 may analyze the result data of the diagnostic device received through the communication part 1200. The result analyzing part 1300 may determine the concentration and/or amount of an object (e.g., antigens or antibodies) that is to be inspected by the diagnostic device through an image analysis.

Specifically, the result analyzing part 1300 may extract a reaction area from the result data of the diagnostic device. Then, the reaction area may mean an area, in which a reagent of the diagnostic device reacts with a specimen. For example, they may be the reaction areas 221 and 223 of FIG. 4B, but are not limited thereto.

The result analyzing part 1300 may generate reaction result data from the reaction area. The reaction result data relates to a form of a reagent reacted by contacting the specimen, and may include a color change of the reagent and the like. The reaction result data may be analysis result data by an artificial intelligence model.

The result analyzing part 1300 may input the reaction result data to the artificial intelligence model Then, the artificial intelligence model may generate healthcare solution data based on an image analysis of the diagnostic device. The health management solution may include a self-diagnosis cycle using an exercise therapy, a diet therapy, and a diagnostic device.

The result analyzing part 1300 may generate user-customized healthcare solution data by additionally input user information to the artificial intelligence model. Accordingly, the result analyzing part 1300 may provide user-customized healthcare solution data in consideration of an underlying disease, a user's environment, eating habits, and exercise habits.

Furthermore, the result analyzing part 1300 may generate defect-related data by identifying whether the diagnostic device is defective and analyzing the cause of the defect. When there is an error in the analysis result based on the result data of the diagnostic device, the result analyzing part 1300 may analyze the cause of the error to generate defect-related data and transmit it to the manufacturer server 3000 through the communication part 1200. The manufacturer may respond to the problem by correcting the manual when it is a problem of misuse by the user by using the defect-related data, or inspecting a production facility when it is a problem of the diagnostic kit.

The usage analyzing part 1400 may analyze and generate data related to the use of the diagnostic device. The usage analyzing part 1400 may generate usage prediction data of the diagnostic device based on user information and healthcare solution data.

Specifically, the usage analyzing part 1400 may calculate the usage of the diagnostic device that the user will use for a certain period of time by using the self-diagnosis cycle included in the health care solution. Because a manufacturer needs a certain period of time to make a diagnostic kit, the manufacturer may reduce the burden of predictive production of the diagnostic device by using the usage prediction data. Furthermore, manufacturers may use usage data to reduce the burden of the effective period and inventory management of the diagnostic device.

The storage part 1500 may store various data and programs that are required to operate the system 1000. The storage part 1500 may store both information acquired by the system 1000 and information processed by the system 1000.

For example, the storage part 1500 may store user information acquired by the communication part 1200 from the user terminal 2000, specification information of the diagnostic device, and result data of the diagnostic device. As another example, the storage part 1500 may store analysis result data generated by the result analyzing part 1300 and the user account analyzing part 1400.

The storage part 1500 may store data temporarily or semi-permanently. For example, the storage part 1500 may be a hard disk drive (HDD), a solid state drive (SSD), a flash memory (ROM), a random access memory (RAM), or cloud storage, but is not limited thereto and may be configured as various modules for storing data.

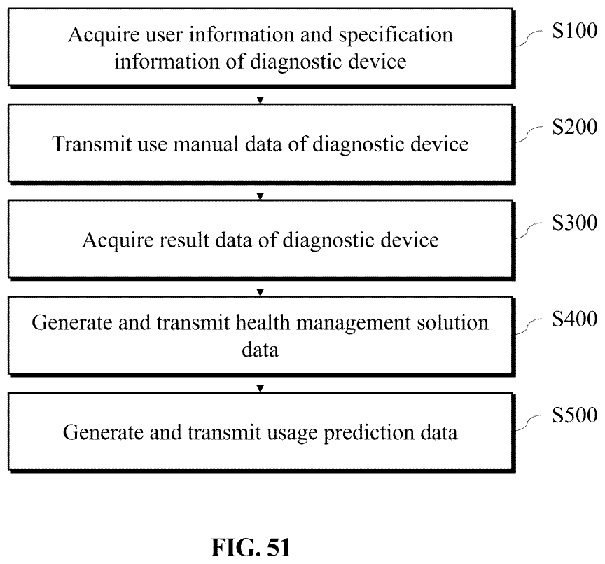

FIG. 51 is a flowchart of a method for providing information related to a diagnostic device according to an embodiment.

Referring to FIG. 51, a method of providing information related to the diagnostic device according to an embodiment may include a operation S100 of acquiring user information and specification information of a diagnostic device, a operation S200 of transmitting use manual data of the diagnostic device, a operation S300 of acquiring result data of the diagnostic device, a operation S400 of generating and transmitting healthcare solution data, and a operation S500 of generating and transmitting usage prediction data.

The operation S100 of acquiring the user information and the specification information of the diagnostic device may be an operation of acquiring the user information and the specification information of the diagnostic device from the user terminal 2000 through the communication part 1200. Then, the user information may include at least one of a gender, an age, an area, a weight, a height, a body mass coefficient, an exercise amount, a sleep amount, information on possessed diseases, information on a medication, an interested diagnostic item, attending hospital information, and a health diagnosis result. Furthermore, the specification information of the diagnostic device may include at least one of a name, a manufacturer, and a possible diagnostic item of the diagnostic device.

The operation S200 of transmitting the use manual data of the diagnostic device may be an operation of transmitting manual data corresponding to the diagnostic device that the user wants to use to the user terminal 2000 based on the specification information of the diagnostic device acquired in operation S100.

The controller 1100 may extract a manual corresponding to the acquired specification information of the diagnostic device from the manual data stored in the storage part 1500. The controller 1100 may transmit the extracted manual data to the user terminal 2000 through the communication part 1200.

For example, when the specification information of the diagnostic device obtained from the user terminal 2000 is a diagnostic device for the coronavirus, the controller 1100 may extract a manual corresponding to the coronavirus diagnostic device from the storage part 1500 and transmit it to the user terminal 2000 through the communication part 1200. As another example, when the specification information of the diagnostic device acquired from the user terminal 2000 is a cortisol hormone diagnostic device, the controller 1100 may extract a manual corresponding to the cortisol hormone diagnostic device from the storage part 1500 and transmit it to the user terminal 2000 through the communication part 1200.

Then, the manual data stored in the storage part 1500 may be updated at all times. Specifically, the communication part 1200 may acquire manual data from an external server and store the manual data in the storage part 1500. Alternatively, the manager of the system 1000 may always add and change manual data to the storage part 1500.

The use manual data of the diagnostic device may include timer information corresponding to the specification information of the diagnostic device. The system 1000 may provide a timer function for guiding a user to use the diagnostic device according to the timer information.

For example, when the acquired specification information of the diagnostic device is a diagnostic device for the coronavirus, the use manual data of the diagnostic device may include timer information of 15 minutes. Accordingly, the system 1000 may notify the user on the provided platform that 15 minutes have elapsed after the specimen was introduced into the diagnostic device. For a specific example, when the user presses a start button of the timer on the platform after the specimen is introduced, the timer on the platform may provide a notification to the user through the user terminal 2000 when 15 minutes have elapsed after the button was pressed.

Additionally, the system 1000 may acquire manufacturing characteristic data of the diagnostic device from the user terminal 2000 and store the data in the storage part 1500. The manufacturing characteristic data may include product characteristic data generated in a final quality inspection in the final stage of the manufacturing process of the diagnostic device. The system 1000 may receive the characteristic of the diagnostic device lot used by the user and correct or adjust the inspection result by using the calibration value and the calibration factor.

For example, when the manufacturing characteristic information of the acquired diagnostic device is 10 points higher than the result value of the same type of product, the system 1000 may correct the current inspection result or adjust the result value by reflecting the above characteristic information.

The operation S300 of acquiring the result data of the diagnostic device may be an operation of acquiring an image acquired by photographing the diagnostic device, into which the specimen is introduced, from the user terminal 2000 through the communication part 1200. Then, the result data of the diagnostic device may be an image captured after a time corresponding to the timer information has exceeded. However, the result data of the diagnostic device is not limited to the image, and may be an image acquired by photographing the diagnostic device. The communication part 1200 may acquire the result data of the diagnostic device and transmit the acquired data to the result analyzing part 1300.

The operation S400 of generating and transmitting healthcare solution data may be an operation of transmitting the healthcare solution data generated by the result analyzing part 1300 to the user terminal 2000 through the communication part 1200. The user terminal 2000 may output an exercise therapy, a diet therapy, a nutritional or health functional food intake guide, digital therapeutics, additional diagnostic test item information, and a self-diagnosis schedule using the diagnostic device on the display based on the received healthcare solution data.

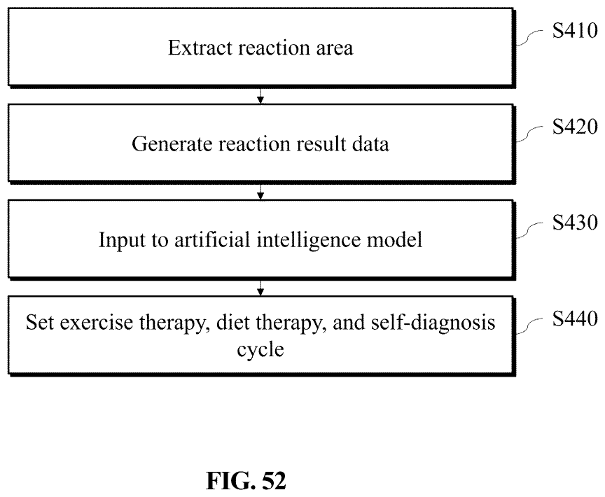

A detailed description of operation S400 will be described later with reference to FIG. 52.

The operation S500 of generating and transmitting usage prediction data may be an operation of the usage averaging part 1400 generating data related to the predicted usage of the diagnostic device based on the user information acquired in operation S100 and the healthcare solution data generated in operation S300 and transmits the data to the manufacturer server 3000.

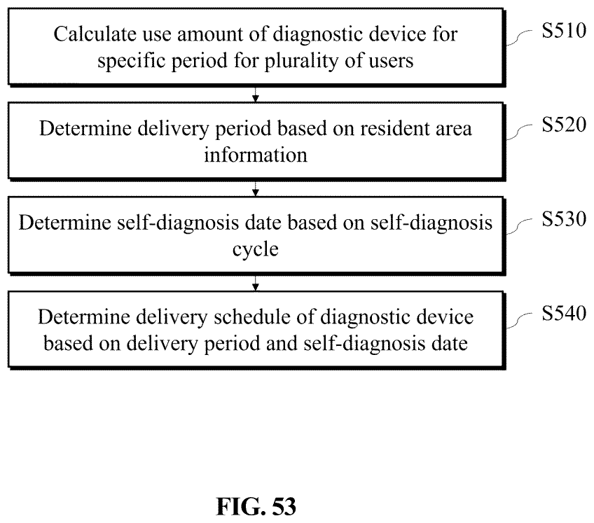

A detailed description of operation S500 will be described later with reference to FIG. 53.

FIG. 52 is a flowchart of a method for generating healthcare solution data according to an embodiment.

Referring to FIG. 52, a method of generating healthcare solution data according to an embodiment may include an operation S410 of extracting a reaction area, an operation S420 of generating reaction result data, an operation S430 of inputting to an artificial intelligence model, an operation S440 of setting an exercise therapy, a diet therapy, a nutritional supplement or health functional food intake guide, a digital therapeutic agent, additional diagnostic test item information, and a self-diagnosis cycle.

The operation S410 of extracting the reaction area may be an operation of extracting an area, in which the reagent of the diagnostic device and the specimen react from the data by the result analyzing part 1300. Then, the result analyzing part 1300 may extract the reaction area based on a position of a predetermined pixel corresponding to the type of the diagnostic device. Alternatively, the result analyzing part 1300 may extract the reaction area based on a result of reacting the substance in the specimen with the sample material. Alternatively, the result analyzing part 1300 may extract the reaction area by using characters (e.g., R, C, T) included in the result data of the diagnostic device.

The operation S420 of generating the reaction result data may be an operation of analyzing the reaction area extracted in operation S410 so that the result analyzing part 1300 generates result data by using an artificial intelligence model. Specifically, the result analyzing part 1300 may generate reaction result data including at least one of negative/positive states and values through a color analysis through a test line extraction from the reaction area and pixel value analysis. The reaction result data may include not only presence of a disease but also the possibility of being affected by other diseases.

The operation S430 of inputting to the artificial intelligence model may be an operation of the result analyzing part 1300 inputting user information and reaction result data to the artificial intelligence model. By inputting user information as well as reaction result data, it is possible to generate user-customized healthcare solution data.

The operation S440 of setting the exercise therapy, the diet therapy, and the self-diagnosis cycle may be an operation of setting an exercise therapy including the user's exercise method, an exercise frequency, an exercise intensity, a diet therapy including nutrients, ingredients, amounts, and recipes necessary for the user, a self-diagnosis cycle, in which the user may identify his or her health condition by using the diagnostic device. Furthermore, in operation S440, a nutritional supplement or health functional food intake guide, by which the user may supply insufficient nutrients, and an additional diagnostic test item may be set so that the user may additionally test the digital therapeutic agent suitable for the user.

The exercise therapy, diet therapy, and self-diagnosis cycle may vary from user to user based on an age, a gender, an underlying disease, and a medication included in the user information. Furthermore, in operation S440, nutritional or health functional food intake guides, digital therapeutics, and additional diagnostic test items may be provided and set in addition to the exercise therapy, the diet therapy, and the self-diagnosis cycle.

The healthcare solution data including the exercise therapy, the diet therapy, the nutritional or health functional food intake guides, the digital therapeutics, the additional diagnostic test item information, and the self-diagnosis cycle set through the above processes may be provided to the user terminal 2000.

The method of generating healthcare solution data may further include an operation of determining a user health pattern based on a plurality of past result data of the diagnostic device and an operation of generating healthcare solution data according to a user health pattern based on current result data of the diagnostic device.

Specifically, the result analyzing part 1400 may determine a user health pattern based on the previously acquired first result data of the diagnostic device, second result data of the diagnostic device, and third result data of the diagnostic device. After determining the pattern, the result analyzing part 1400 may acquire fourth result data of the diagnostic device, which is current result data of the diagnostic device. The result analyzing part 1400 may identify what pattern the user is currently in based on the fourth result data of the diagnostic device and the user health pattern, and generate healthcare solution data accordingly.

A diagnostic device related to an ovulation will be described as an example. For example, the result analyzing part 1400 may set an ovulation pattern of the user based on the first to third result data of the diagnostic device. Then, the first to third result data of the diagnostic device may be a result of diagnosis on different days. In an example, an ovulation pattern is determined using three result data of the diagnostic device, but the number of the result data of the diagnostic device for setting an ovulation pattern is not limited thereto.

After setting the ovulation pattern, the result analyzing part 1400 may acquire the result data of the fourth diagnostic device, which is the current data. As a result of analyzing the result data of the fourth diagnostic device, when it is identified that the value of item “A” is 70%, the result analyzing part 1400 may identify a state, in which the value of item A is 70% in the ovulation pattern, as the current state of the user. The result analyzing part 1400 may provide the user with a health management solution including the exercise therapy, the diet therapy, and the self-diagnosis cycle based on the identified current state of the user. Furthermore, by transmitting data on the set ovulation pattern and the current state of the user to the fertility center, a cooperation with the infertility center may be performed.

Furthermore, a diagnostic device related to a cortisol hormone, which is a hormone related to stress, will be described as an example. For example, the result analyzing part 1400 may set a stress pattern of the user based on the first to third result data of the diagnostic device. Then, the first to third result data of the diagnostic device may be a result of diagnosis in morning, lunch, and evening for one day. In an example, a stress pattern is determined by using three result data of the diagnostic device, but the number of result data of the diagnostic device for setting a stress pattern is not limited thereto.

After setting the stress pattern, the resultant analyzing part 1400 may acquire the fourth result data of the diagnostic device which is the current data. As a result of analyzing the fourth result data of the diagnostic device, the resultant analyzing part 1400 may identify the current state of the user in the stress pattern when it is identified that the level of cortisol hormone is “B”. Specifically, the resultant analyzing part 1400 may identify whether the user is a Cushing syndrome or an adrenocorticotropic syndrome using the stress pattern. The resultant analyzing part 1400 may provide a health care solution including the exercise therapy, the diet therapy, the nutritional supplements or health functional food intake guide, digital therapeutic agents, additional diagnostic test item information, and self-diagnosis cycle to the user based on the identified current state of the user. Furthermore, by transmitting data on the set stress pattern and the current state of the user to the consulting center, a cooperation with the consulting center may be performed.

Furthermore, the glycated hemoglobin diagnostic device, which is an item used for diagnosing diabetes, is described as an example. For example, the result analyzing part 1400 may set a glycated hemoglobin change pattern of the user based on the first to third result data of the diagnostic device. Then, the first to third result data of the diagnostic device may be a diagnosis result of two weeks, one month, or two or more months. In an example, a glycated hemoglobin change pattern is determined by using single or two or more result data of the diagnostic device, but the number of result data of the diagnostic device for setting a glycated hemoglobin change pattern is not limited thereto.

After setting the glycated hemoglobin change pattern, the resultant analyzing part 1400 may acquire the fourth result data of the diagnostic device, which is the current data. The resultant analyzing part 1400 may determine the current state of the user using the glycated hemoglobin change pattern when it is determined that the glycated hemoglobin level is “C” as a result of analyzing the fourth result data of the diagnostic device. Specifically, the resultant analyzing part 1400 may determine whether the user has an increased or decreased risk of diabetes by using the glycated hemoglobin change pattern. The resultant analyzing part 1400 may provide a health care solution including the exercise therapy, the diet therapy, the nutritional supplements or health functional food intake guide, digital therapeutic agents, additional diagnostic test item information, and self-diagnosis cycle to the user based on the identified current state of the user. Furthermore, data on the set glycated hemoglobin change pattern and the user's current state may be transmitted to a diabetes-related counseling center to perform consultation with the counseling center.

Furthermore, a diagnostic device used for diagnosing vitamin “D” is described as an example. For example, the result analyzing part 1400 may set a vitamin “D” concentration change pattern of the user based on the first to third result data of the diagnostic device. Then, the first to third result data of the diagnostic device may be a diagnosis result of two weeks, one month, or two or more months. In an example, a vitamin “D” concentration change pattern is determined using single or two or more result data of the diagnostic device, but the number of result data of the diagnostic device for setting a vitamin “D” concentration change pattern is not limited thereto.

After setting the vitamin “D” concentration change pattern, the resultant analyzing part 1400 may acquire the fourth result data of the diagnostic device, which is the current data. As a result of analyzing the fourth result data of the diagnostic device, the resultant analyzing “D” level when it is identified that the vitamin “D” level is “D”. Specifically, the resultant analyzing part 1400 may determine whether the risk of the user is increasing or decreasing due to the vitamin “D” deficiency or deficiency using the vitamin “D” concentration change pattern. The resultant analyzing part 1400 may provide a health care solution including the exercise therapy, the diet therapy, the nutritional supplements or health functional food intake guide, digital therapeutic agents, additional diagnostic test item information, and self-diagnosis cycle to the user based on the identified current state of the user. Furthermore, by transmitting data on the set vitamin “D” concentration change pattern and the user's current state to a health-related counseling center, consultation with the counseling center may be performed.

In the operation of generating healthcare solution data, data of an external device may be used in addition to user information and reaction result data. Then, the external device may include a wearable device (a smart watch or a smart ring), a smart thermometer, a smart scale, and the like. For example, the data of the external device may be objectively reliable data, such as a blood pressure, a heart rate, a body temperature, a weight, the number of steps, and an exercise amount. The result analyzing part 1300 may generate healthcare solution data by inputting user information, reaction result data, and data of an external device into an artificial intelligence model.

FIG. 53 is a flowchart of a method for generating usage prediction data according to an embodiment.

Referring to FIG. 53, a method of generating usage prediction data according to an embodiment may include an operation S510 of calculating a diagnostic device usage amount for a specific period for a plurality of users, an operation S520 of determining a delivery period based on residential area information, an operation S530 of determining a self-diagnosis date based on a self-diagnosis cycle, and an operation S540 of determining a diagnostic device delivery schedule based on a delivery period and a self-diagnosis date.

The operation S410 of calculating a diagnostic device usage amount for a specific period for a plurality of users may be an operation of calculating a predicted usage amount of the diagnostic device based on the self-diagnosis cycle included in the health management solution. The usage analyzing part 1400 may calculate a diagnostic device usage amount for a specific period based on the self-diagnosis cycle using the diagnostic device predicted for each user. Furthermore, the usage analyzing part 1400 may calculate a diagnostic device usage amount for a specific period for a plurality of users based on the predicted usage amount of the diagnostic device calculated for each user.

For example, when the first user's self-diagnosis cycle is 2 weeks and the second user's self-diagnosis cycle is 10 days, the usage analyzing part 1400 may calculate that the predicted usage of the first user is 2 and the predicted usage of the second user is 3. Accordingly, the usage analyzing part 1400 may determine that five diagnostic devices are required for two users during November.

The operation S520 of determining the delivery period based on the residential area information may be an operation of the usage analyzing part 1400 identifying different delivery periods according to the residential area of the user. For example, when the residential area of the first user is Seoul and the residential area of the second user is Jeju Island, the usage analyzing part 1400 may set the delivery period for the first user to three days and the delivery period for the second user to seven days.

The operation S530 of determining the self-diagnosis date based on the self-diagnosis cycle may be an operation of checking the delivery period determined in operation S520 by the usage analyzing part 1400 and the self-diagnosis cycle to be diagnosed by the user. In the above example, the self-diagnosis days of the first user who has to use two in November may be 7 days and 21 days, and the self-diagnosis days of the second user who has to use three in November may be 10 days, 20 days, and 30 days.

The operation S540 of determining the delivery schedule of the diagnostic device based on the delivery period and the self-diagnosis date may determine a delivery schedule to be delivered by the manufacturer based on the delivery period and self-diagnosis date determined in operations S520 and S530. In the above example, the usage analyzing part 1400 may set the delivery date of the diagnostic device to the first user to the 4th and 18th days, and set the delivery date of the diagnostic device to the second user to the 3rd, 13th, and 23rd days. Alternatively, the usage analyzing part 1400 may set the delivery date of the diagnostic device to the first user to the 4th of each month, and set the delivery date of the diagnostic device to the second user to the 3rd of each month.

The usage analyzing part 1400 may transmit usage prediction data of the diagnostic device including a diagnostic device delivery schedule to the manufacturer server 3000. The manufacturer may establish a production schedule of the diagnostic device based on the usage prediction data of the diagnostic device.

The method according to an embodiment may be configured in the form of program instructions that may be executed through various computer means and recorded on a computer-readable medium. The computer-readable medium may include program instructions, data files, data structures, and the like alone or in combination. Program instructions recorded on the medium may be specially designed and configured for embodiments, or may be known to and usable by those skilled in computer software. Examples of computer-readable recording media include magnetic media such as hard disks, floppy disks and magnetic tapes, optical media such as CD-ROMs and DVDs, magnetic-optical media such as planetary disks, and hardware devices specifically configured to store and perform program instructions such as ROMs, RAMs, and flash memories. Examples of program instructions include not only mechanical codes such as those made by compilers, but also advanced language codes that may be executed by computers using interpreters or the like. The hardware device described above may be configured to operate as one or more software modules to perform the operations of the embodiment, and vice versa.

According to an embodiment of the present disclosure, a method capable of providing information related to a diagnostic device to a user terminal and a manufacturer server may be provided.

Although the embodiments have been described by limited embodiments and drawings as described above, various modifications and modifications may be made from the above description by those of ordinary skill in the art. For example, appropriate results may be achieved even when the described techniques are performed in a different order from the described method, and/or components such as systems, structures, devices, circuits, etc. are combined or combined in a different form from the described method, or replaced or replaced by other components or equivalents.

Accordingly, other implementations, other embodiments, and those equivalent to the claims fall within the scope of the claims described below.

What is claimed is:

1. An in vitro diagnostic device comprising:
a housing including a first hole and a second hole, wherein the first hole is configured to receive a specimen; and
at least one pad including a first pad, of which at least a partial area is exposed to an outside through the second hole,
wherein the first pad exposed to the outside through the second hole is configured to include a first reaction area, a second reaction area, and at least one sign, and
wherein at least one of the first reaction area or the second reaction area is configured to express a reaction result based on a reaction with the specimen.

2. The in vitro diagnostic device of claim 1, wherein at least one of the first reaction area or the second reaction area is configured to express a control line or a test line, and
wherein the at least one sign includes a plurality of color signs.

3. The in vitro diagnostic device of claim 2, wherein a height from a lower surface of the in vitro diagnostic device to at least one of the first reaction area or the second reaction area is a first height,
wherein a height from the lower surface of the in vitro diagnostic device to the at least one sign is a second height, and
wherein the first height and the second height correspond to each other.

4. The in vitro diagnostic device of claim 3, wherein a height from the lower surface of the in vitro diagnostic device to an outer surface of at least a portion of the housing is a third height, and
wherein the third height is different from the first height and the second height.

5. The in vitro diagnostic device of claim 4, wherein at least partial area of the first pad exposed to the outside through the second hole includes a plurality of areas divided by the first reaction area and the second reaction area, and
wherein the at least one color sign is formed at least a portion of the plurality of areas.

6. The in vitro diagnostic device of claim 2, wherein colors of the plurality of color signs have colors associated with the first reaction area or the second reaction area, and at least one of brightnesses and chromas thereof is different.

7. The in vitro diagnostic device of claim 1, wherein the at least one pad includes a second pad being different from the first pad, and
wherein the second pad includes at least one sign.

8. The in vitro diagnostic device of claim 7, wherein a height from the lower surface of the in vitro diagnostic device to the first pad is a fourth height,
wherein a height from the lower surface of the in vitro diagnostic device to the second pad is a fifth height, and
wherein the fourth height and the fifth height correspond to each other.

9. The in vitro diagnostic device of claim 1, wherein the at least one pad includes a plurality of pads, of which heights from the lower surface of the in vitro diagnostic device are the same, and
wherein at least one sign is formed for each of the plurality of pads.

10. The in vitro diagnostic device of claim 9, wherein the plurality of pads are configured to analyze biometric information measurement values of the same kind.

11. An in vitro diagnostic device comprising:
a housing including a plurality of holes; and
a plurality of pads, of which at least partial areas are exposed to an outside through corresponding ones of the plurality of holes, wherein heights of the plurality of pads from a lower surface of the in vitro diagnostic device correspond to each other,
the at least partial areas of the plurality of pads exposed to the outside is configured to include a first reaction area, a second reaction area, and a plurality of color signs, and
wherein at least one of the first reaction area or the second reaction area is configured to express a reaction result based on a reaction with a specimen.
